(19) United States
(12) Patent Application Publication
(10) Pub. No.: US 2025/0346888 A1
(43) Pub. Date: Nov. 13, 2025

(54) PROMOTER PROXIMAL SEQUENCES AND USES THEREOF FOR RNA MANUFACTURING

(71) Applicant: Helix Nanotechnologies Inc, Boston, MA (US)

(72) Inventors: Arianna Lizeth Lopez Sauceda, Boston, MA (US); Nikolai Eroshenko, Boston, MA (US); Nikhil Dhar, Boston, MA (US)

(21) Appl. No.: 19/272,607

(22) Filed: Jul. 17, 2025

Related U.S. Application Data
(63) Continuation of application No. PCT/US24/42648, filed on Aug. 16, 2024.
(60) Provisional application No. 63/520,597, filed on Aug. 18, 2023.

(51) Int. Cl. C12N 15/11; A61K 31/7105; C12N 9/12; C12P 19/34

(52) U.S. Cl. CPC C12N 15/11; A61K 31/7105; C12N 9/1247; C12P 19/34; C12Y 207/07006

(57) ABSTRACT

Disclosed herein are polynucleotides comprising promoter proximal sequences for use in producing polyribonucleotides with increased yield and purity. Also provided herein are compositions comprising the disclosed polynucleotides, and methods of making and using the same.

### CROSS REFERENCE TO RELATED APPLICATIONS

This application claims priority to and the benefit of U.S. Provisional Patent Application No. 63/520,597 filed on Aug. 18, 2023, the entire contents of which are hereby incorporated by reference in its entirety.

### BACKGROUND

Development of RNA as a therapeutic platform has seen large progress. However, to fully realize the potential of RNA therapeutics, there is still much progress to be made on further improvements particularly relating to RNA manufacturing and scalability.

### SUMMARY

The present disclosure recognizes that manufacturing RNA, e.g., for therapeutic applications, at a desired yield and/or purity has been challenging. The present disclosure further recognizes that scaling up RNA manufacturing, e.g., to produce sufficient RNA for therapeutic applications, is particularly challenging. Indeed, increasing the yield, purity and/or scalability of RNA manufacturing often comes at the price of increasing manufacturing costs which in turn hampers development and/or use of RNA as a therapeutic.

Accordingly, the present disclosure provides solutions to this challenge with polynucleotide constructs disclosed herein that increase the yield and/or purity of polyribonucleotides produced by the disclosed polynucleotide constructs. Using polynucleotide constructs disclosed herein, polyribonucleotides having increased yield and/or purity can be made thus reducing manufacturing costs. Additionally, methods disclosed herein can be used to produce polyribonucleotides with higher quality that can be used for repeated dosing applications. Polynucleotide constructs disclosed herein further allow for the production of polyribonucleotides at increased yield while maintaining purity of the polyribonucleotides. Polynucleotides disclosed herein comprise two or more identical consecutive nucleotides at the 3′ end (e.g., downstream) of a promoter sequence, e.g., two or more adenine, two or more cytosines, and/or two or more thymines. In some embodiments, two or more identical consecutive nucleotides are positioned downstream of a promoter sequence and upstream of a sequence encoding a target, e.g., a payload.

Provided herein is a recombinant polynucleotide sequence, comprising: (i) a promoter sequence comprising a 5′ end and a 3′ end; (ii) a promoter proximal sequence adjacent to the 3′ end of the promoter sequence, wherein the promoter proximal sequence comprises two or more consecutive nucleotides; and (iii) a sequence encoding a target operably linked to (ii).

In some embodiments, two or more consecutive nucleotides are the same nucleotide.

In some embodiments, two or more consecutive nucleotides form exactly two or three hydrogen bonds when paired with a complementary nucleotide.

In some embodiments, when paired with a complementary sequence of nucleotides, the promoter proximal sequence has a lower melting temperature than a reference promoter proximal sequence.

In some embodiments, a recombinant polynucleotide has a lower persistence length than a comparable reference polynucleotide, wherein a reference polynucleotide is identical to a recombinant polynucleotide except that a reference polynucleotide comprises a reference promoter proximal sequence instead of a promoter proximal sequence.

In some embodiments, a reference promoter proximal sequence comprises the same number of nucleotides compared to a promoter proximal sequence.

In some embodiments, a reference promoter proximal sequence comprises a fewer number of nucleotides compared to a promoter proximal sequence.

In some embodiments, a ratio of guanine/cytosine to adenine/thymine in a reference promoter proximal sequence is 1:1 or greater.

In some embodiments, a two or more consecutive nucleotides in a promoter proximal sequence are: adenine or a non-natural variant thereof, thymine or a non-natural variant thereof, cytosine or a non-natural variant thereof, or combinations thereof.

In some embodiments, a comparable reference promoter proximal sequence comprises only guanine.

In some embodiments, a comparable reference promoter proximal sequence comprises a lesser number of consecutive: adenine or a non-natural variant thereof, thymine or a non-natural variant thereof, or cytosine or a non-natural variant thereof, as compared to a promoter proximal sequence.

In some embodiments, a promoter proximal sequence comprises adenines, thymines, cytosines, or any combination thereof.

In some embodiments, a promoter proximal sequence comprises two or more consecutive adenines. In some embodiments, a promoter proximal sequence comprises two or more consecutive thymines. In some embodiments, a promoter proximal sequence comprises two or more consecutive cytosines.

In some embodiments, a promoter proximal sequence is at least 3 nucleotides in length. In some embodiments, a promoter proximal sequence is at least about 3 nucleotides, at least about 4 nucleotides, at least about 5 nucleotides, at least about 6 nucleotides, at least about 7 nucleotides, at least about 8 nucleotides, at least about 9 nucleotides, at least about 10 nucleotides, at least about 11 nucleotides, at least about 12 nucleotides, at least about 13 nucleotides, at least about 14 nucleotides, at least about 15 nucleotides, at least about 16 nucleotides, at least about 17 nucleotides, at least about 18 nucleotides, at least about 19 nucleotides, or at least about 20 nucleotides in length.

In some embodiments, a promoter proximal sequence comprises adenines. In some embodiments, a promoter proximal sequence comprises at least 3 adenines, at least 4 adenines, at least 5 adenines, at least 6 adenines, at least 7 adenines, at least 8 adenines, at least 9 adenines, at least 10 adenines, at least 11 adenines, at least 12 adenines, at least 13 adenines, at least 14 adenines, or at least 15 adenines.

In some embodiments, a promoter proximal sequence comprises thymines. In some embodiments, a promoter proximal sequence comprises at least 3 thymines, at least 4 thymines, at least 5 thymines, at least 6 thymines, at least 7 thymines, at least 8 thymines, at least 9 thymines, at least 10 thymines, at least 11 thymines, at least 12 thymines, at least 13 thymines, at least 14 thymines, or at least 15 thymines.

In some embodiments, a promoter proximal sequence comprises cytosines. In some embodiments, a promoter proximal sequence comprises at least 3 cytosines, at least 4 cytosines, at least 5 cytosines, at least 6 cytosines, at least 7 cytosines, at least 8 cytosines, at least 9 cytosines, at least 10 cytosines, at least 11 cytosines, at least 12 cytosines, at least 13 cytosines, at least 14 cytosines, or at least 15 cytosines.

In some embodiments, a promoter is an RNA polymerase promoter.

In some embodiments, a promoter is a bacteriophage promoter, a viral promoter, a bacterial promoter, a eukaryotic promoter or an engineered promoter.

In some embodiments, a bacteriophage promoter is a T7 promoter or a variant or a fragment thereof, a T3 promoter or a variant or a fragment thereof, or an SP6 promoter or a variant or a fragment thereof.

In some embodiments, a T7 promoter comprises an AGG sequence downstream of a TATA sequence. In some embodiments, a T7 promoter comprises the sequence of SEQ ID NO: 6.

In some embodiments, a T7 promoter comprises a GGG sequence downstream of a TATA sequence. In some embodiments, a T7 promoter comprises the sequence of SEQ ID NO: 70.

In some embodiments, a polynucleotide comprising a T7 promoter comprises the sequence of SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15 or SEQ ID NO: 16.

In some embodiments, a polynucleotide comprising a T7 promoter comprises the sequence of SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO:79 or SEQ ID NO: 80.

In some embodiments, a T3 promoter comprises the sequence of SEQ ID NO: 17.

In some embodiments, a polynucleotide comprising a T3 promoter comprises the sequence of SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, or SEQ ID NO: 26.

In some embodiments, a T3 promoter comprises the sequence of SEQ ID NO: 81.

In some embodiments, a polynucleotide comprising a T3 promoter comprises the sequence of SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89 or SEQ ID NO: 90.

In some embodiments, a SP6 promoter comprises the sequence of SEQ ID NO: 27 or SEQ ID NO: 60.

In some embodiments, a polynucleotide comprising an SP6 promoter comprises the sequence of SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 61, SEQ ID NO: 62, SEQ ID NO: 63, SEQ ID NO: 64, SEQ ID NO: 65, SEQ ID NO: 66, SEQ ID NO:67, SEQ ID NO: 68 or SEQ ID NO: 69.

In some embodiments, a sequence encoding the target is situated 3′ of the promoter proximal sequence.

In some embodiments, a target comprises a polyribonucleotide, e.g., an RNA oligo; a messenger RNA; a gRNA; an inhibitory RNA; an miRNA or siRNA; an antisense oligonucleotide; a long-non-coding RNA, a circular RNA, or any combination thereof.

In some embodiments, a polynucleotide disclosed herein further comprises one or more additional elements. In some embodiments, one or more additional elements comprises one or more UTRs, a polyadenylation signal sequence, or combinations thereof.

Further provided herein is a method making a polyribonucleotide, comprising a step of incubating a transcription mixture comprising: (i) a recombinant polynucleotide disclosed herein; (ii) at least one RNA polymerase that recognizes a promoter sequence; and (iii) a plurality of ribonucleotides comprising at least two different types of ribonucleotides, each type comprising a different nucleoside; thereby producing the polyribonucleotide.

In some embodiments, a polyribonucleotide produced by a method disclosed herein is an in vitro transcribed polyribonucleotide.

In some embodiments, a polyribonucleotide is suitable for use as a therapeutic. In some embodiments, a therapeutic is used in: (i) a method to stimulate an immune response; (ii) an antibody therapy method; (iii) an immune-modulation method; (iv) a vaccination method; (v) a gene therapy method; (vi) a cell therapy engineering method; (vii) an immunotherapy method; (viii) a protein replacement therapy method; (ix) a chemotherapeutic method; or (x) a combination of (i)-(ix).

In some embodiments, a polyribonucleotide is produced at a higher yield compared to an otherwise similar polyribonucleotide made with a DNA template without a promoter proximal sequence or with a different promoter proximal sequence.

In some embodiments, a yield is at least about 1.5-fold higher, at least about 2-fold higher, 2.5-fold higher, at least about 3-fold higher, at least about 4-fold higher, at least about 5-fold higher, at least about 6-fold higher, at least about 7-fold higher, at least about 8-fold higher, at least about 9-fold higher, at least about 10-fold higher, at least about 20-fold higher, or at least about 50-fold higher.

In some embodiments, a polyribonucleotide is characterized in that when administered to a cell, tissue, or subject:
(i) the polyribonucleotide is expressed and/or translated at a substantially similar level when compared to expression and/or translation of an otherwise similar polyribonucleotide made with a DNA template without a promoter proximal sequence or with a different promoter proximal sequence that is administered to the cell, tissue, or subject; and/or
(ii) a substantially similar viability is observed as compared to the viability of a cell, tissue, or subject administered an otherwise similar polyribonucleotide made with a DNA template without a promoter proximal sequence or with a different promoter proximal sequence.

In some embodiments, a polyribonucleotide is characterized in that when administered to a cell, tissue, or subject, the polyribonucleotide has similar immunogenicity when compared to immunogenicity of an otherwise similar polyribonucleotide made with a DNA template without a promoter proximal sequence or with a different promoter proximal sequence that is administered to the cell, tissue, or subject.

In some embodiments, a polyribonucleotide is characterized in that when administered to a cell, tissue, or subject, the polyribonucleotide has reduced immunogenicity when compared to immunogenicity of an otherwise similar polyribonucleotide made with a DNA template without a promoter proximal sequence or with a different promoter proximal sequence that is administered to the cell, tissue, or subject.

Also provided herein is a polyribonucleotide made according to a method disclosed herein.

This disclosure further provides a pharmaceutical composition comprising a polyribonucleotide made according to a method disclosed herein.

Further provided herein is a method comprising administering: a polyribonucleotide made according to a method disclosed herein, or a pharmaceutical composition disclosed herein, wherein the method is: (i) a method to stimulate an immune response; (ii) an antibody therapy method; (iii) an immune-modulation method; (iv) a vaccination method; (v) a gene therapy method; (vi) a cell therapy engineering method; (vii) an immunotherapy method; (viii) a protein replacement therapy method; (ix) a chemotherapeutic method; or (x) a combination of (i)-(ix).

This disclosure provides use of a polyribonucleotide made according to a method disclosed herein, or a pharmaceutical composition disclosed herein in the preparation of a medicament for: (i) a method to stimulate an immune response; (ii) an antibody therapy method; (iii) an immune-modulation method; (iv) a vaccination method; (v) a gene therapy method; (vi) a cell therapy engineering method; (vii) an immunotherapy method; (viii) a protein replacement therapy method; (ix) a chemotherapeutic method; or (x) a combination of (i)-(ix).

Provided herein is a composition comprising a polyribonucleotide made according to a method disclosed herein, or a pharmaceutical composition disclosed herein, for use in: (i) a method to stimulate an immune response; (ii) an antibody therapy method; (iii) an immune-modulation method; (iv) a vaccination method; (v) a gene therapy method; (vi) a cell therapy engineering method; (vii) an immunotherapy method; (viii) a protein replacement therapy method; (ix) a chemotherapeutic method; or (x) a combination of (i)-(ix).

In some embodiments, polyribonucleotide made according to a method disclosed herein comprises one or more modified ribonucleotides comprising: a modified nucleobase, a modified ribose, a modified backbone, or any combination thereof.

In some embodiments, one or more modified ribonucleotides has: a 5′ monophosphate; a 5′ diphosphate; or a 5′ triphosphate. In some embodiments, one or more modified ribonucleotides comprises a 5′ triphosphate.

In some embodiments, one or more modified ribonucleotides comprises a nucleoside comprising an acetyl group, wherein the nucleoside is N4-acetylcytidine. In some embodiments, one or more modified ribonucleotides has a structure provided in Table 4. In some embodiments, one or more modified ribonucleotides has a structure provided in Table 5 when incorporated in a polynucleotide (e.g., polyribonucleotide). In some embodiments, a polynucleotide (e.g., polyribonucleotide) comprises one or more modified ribonucleotides that have a structure provided in Table 5.

In some embodiments, the nucleoside has a structure of:

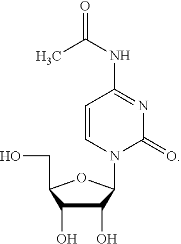

In some embodiments, one or more modified ribonucleotides has the structure of:

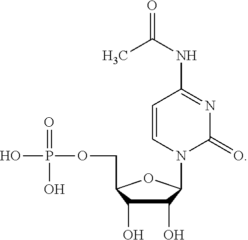

In some embodiments, one or more modified ribonucleotides has the structure of:

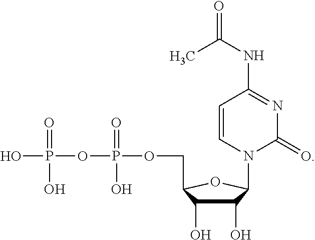

In some embodiments, one or more modified ribonucleotides has the structure of:

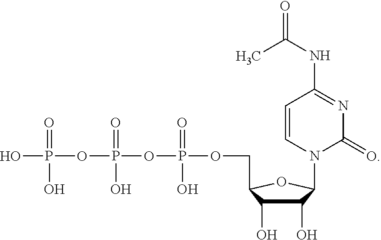

In some embodiments, a polynucleotide (e.g., polyribonucleotide) comprises one or more modified ribonucleotides that have the following structure:

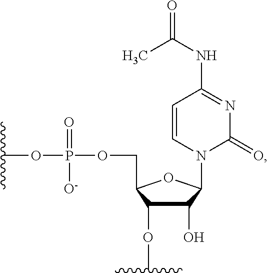

wherein [symbol] indicates the position of attachment to an adjacent ribonucleotide.

In some embodiments, a polynucleotide (e.g., polyribonucleotide) comprises one or more modified ribonucleotides that have the following structure:

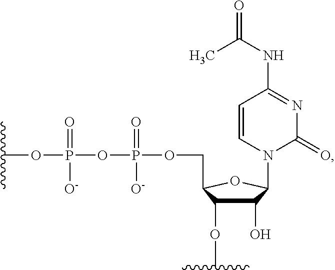

wherein [symbol] indicates the position of attachment to an adjacent ribonucleotide.

In some embodiments, a polynucleotide (e.g., polyribonucleotide) comprises one or more modified ribonucleotides that have the following structure:

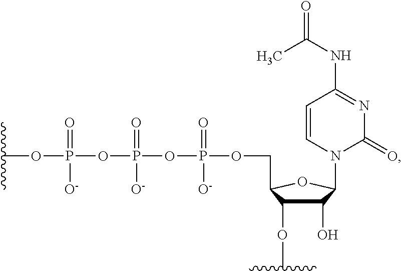

wherein [symbol] indicates the position of attachment to an adjacent ribonucleotide.

In some embodiments, a polyribonucleotide comprises cytidine nucleosides, and at least 5% of cytidine nucleosides in a polyribonucleotide comprise N4-acetylcytidine.

In some embodiments, a polyribonucleotide comprises cytidine nucleosides, and less than 100% of cytidine nucleosides in the polyribonucleotide comprise N4-acetylcytidine.

In some embodiments, a polyribonucleotide comprises cytidine nucleosides, and at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% of cytidine nucleosides in a polyribonucleotide comprise N4-acetylcytidine.

In some embodiments, one or more modified ribonucleotides comprises a nucleoside comprising a hydroxymethyl group, wherein the nucleoside is 5-hydroxymethyluridine. In some embodiments, one or more modified ribonucleotides has a structure provided in Table 4. In some embodiments, one or more modified ribonucleotides has a structure provided in Table 5 when incorporated in a polynucleotide (e.g., polyribonucleotide). In some embodiments, a polynucleotide (e.g., polyribonucleotide) comprises one or more modified ribonucleotides that have a structure provided in Table 5.

In some embodiments, the nucleoside has a structure of:

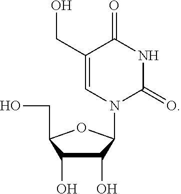

In some embodiments, one or more modified ribonucleotides has a structure of:

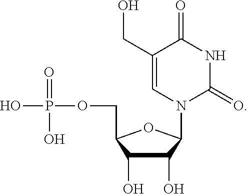

In some embodiments, one or more modified ribonucleotides has a structure of:

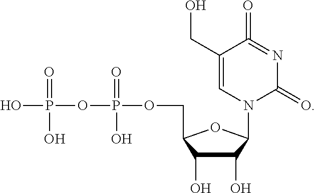

In some embodiments, one or more modified ribonucleotides has a structure of:

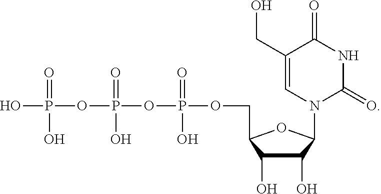

In some embodiments, a polynucleotide (e.g., polyribonucleotide) comprises one or more modified ribonucleotides that have the following structure:

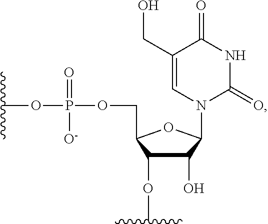

wherein [symbol] indicates the position of attachment to an adjacent ribonucleotide.

In some embodiments, a polynucleotide (e.g., polyribonucleotide) comprises one or more modified ribonucleotides that have the following structure:

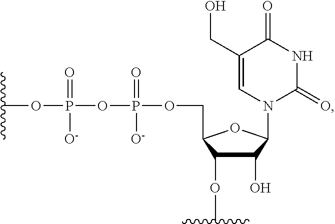

wherein [symbol] indicates the position of attachment to an adjacent ribonucleotide.

In some embodiments, a polynucleotide (e.g., polyribonucleotide) comprises one or more modified ribonucleotides that have the following structure:

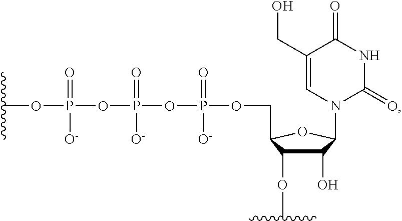

wherein [symbol] indicates the position of attachment to an adjacent ribonucleotide.

In some embodiments, polyribonucleotide made according to a method disclosed herein comprises uridine nucleosides and at least 5% of uridine nucleosides in the polyribonucleotide comprise 5-hydroxymethyluridine.

In some embodiments, a polyribonucleotide comprises uridine nucleosides and less than 100% of uridine nucleosides in the polyribonucleotide comprise 5-hydroxymethyluridine.

In some embodiments, a polyribonucleotide comprises uridine nucleosides and at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% of uridine nucleosides in a polyribonucleotide comprise 5-hydroxymethyluridine.

In some embodiments, a polyribonucleotide comprises uridine nucleosides and more than 60% of uridine nucleosides in a polyribonucleotide comprise 5-hydroxymethyluridine.

In some embodiments, polyribonucleotide made according to a method disclosed herein comprises one or modified nucleotides. In some embodiments, one or more modified ribonucleotides comprises: N4-acetylcytidine, 5-hydroxymethyluridine, N1-methylpseudouridine, pyridin-4-one ribonucleoside, 5-aza-uridine, 6-aza-uridine, 2-thio-5-aza-uridine, 2-thio-uridine (s2U), 5-methyl cytidine (m5C), 5-aza-cytidine, 6-aza-cytidine, pseudoisocytidine, 3-methyl-cytidine (m3C), 5-formyl-cytidine (f5C), N4-methyl-cytidine (m4C), 2-amino-purine, 2, 6-diaminopurine, 2-amino-6-halo-purine, 6-halo-purine, inosine (I), 1-methyl-inosine (m1 I), wyosine (imG), methylwyosine (mimG), 5-hydroxycytidine, 5-hydroxymethylcytidine, 5-carboxycytidine, 5-methoxycytidine, 5-propynylcytidine, 2-thiocytidine, 5-hydroxyuridine, 5-methyluridine, 5,6-dihydro-5-methyluridine, 2′-O-methyluridine, 2′-O-methyl-5-methyluridine, 2′-fluoro-2′-deoxyuridine, 2′-amino-2′-deoxyuridine, 2′-azido-2′-deoxyuridine, 4-thiouridine, 5-carboxyuridine, 5-carboxymethylesteruridine, 5-formyluridine, 5-methoxyuridine, 5-propynyluridine, 5-bromouridine, 5-iodouridine, 5-fluorouridine, pseudouridine, 2′-O-methyl-pseudouridine, N1-hydroxypseudouridine, 2′-O-methyl-N1-methylpseudouridine, N1-ethylpseudouridine, N1-hydroxymethylpseudouridine, ara-uridine, N6-methyladenosine, 2-aminoadenosine, 3-methyladenosine, 7-deazaadenosine, 8-oxoadenosine, thienoguanosine, 7-deazaguanosine, 8-oxoguanosine, 6-O-methylguanine, or any combination thereof. In some embodiments, one or more modified ribonucleotides comprises a nucleoside comprising a ribose moiety comprising an acetyl group, wherein the ribose is 2′-O-acetylated.

In some embodiments, one or more modified ribonucleotide has a structure of:

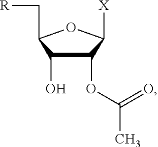

(a) wherein R is a monophosphate, a diphosphate, or a triphosphate; and
(b) wherein X is an adenine nucleobase, a guanine nucleobase, a cytosine nucleobase (e.g., N4-acetylcytosine), or a uracil nucleobase (e.g., 5-hydroxymethyluracil).

In some embodiments, one or more modified ribonucleotides comprises: (i) a 2′-O-acetylated ribose and (ii) an adenine nucleobase. In some embodiments, one or more modified ribonucleotides has a structure provided in Table 6. In some embodiments, one or more modified ribonucleotides has a structure provided in Table 7 when incorporated in a polynucleotide. In some embodiments, a polynucleotide (e.g., a polyribonucleotide) comprises one or more modified ribonucleotides that have a structure provided in Table 7.

In some embodiments, a polynucleotide (e.g., polyribonucleotide) comprises one or more modified ribonucleotides that have the following structure:

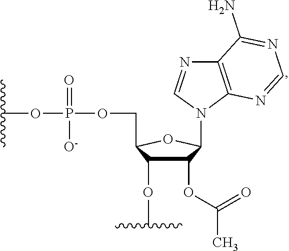

wherein [symbol] indicates the position of attachment to an adjacent ribonucleotide.

In some embodiments, a polynucleotide (e.g., polyribonucleotide) comprises one or more modified ribonucleotides that have the following structure:

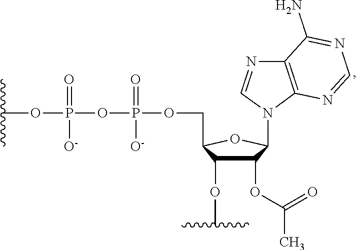

wherein [symbol] indicates the position of attachment to an adjacent ribonucleotide.

In some embodiments, a polynucleotide (e.g., polyribonucleotide) comprises one or more modified ribonucleotides that have the following structure:

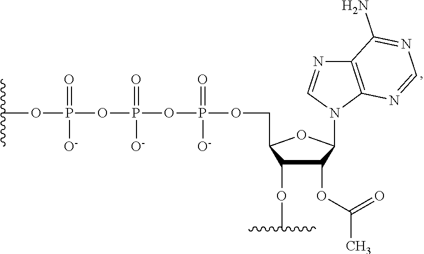

wherein [symbol] indicates the position of attachment to an adjacent ribonucleotide.

In some embodiments, one or more modified ribonucleotides comprises: (i) a 2′-O-acetylated ribose and (ii) a guanine nucleobase. In some embodiments, one or more modified ribonucleotides has a structure provided in Table 6. In some embodiments, one or more modified ribonucleotides has a structure provided in Table 7 when incorporated in a polynucleotide. In some embodiments, a polynucleotide (e.g., a polyribonucleotide) comprises one or more modified ribonucleotides that have a structure provided in Table 7.

In some embodiments, a polynucleotide (e.g., polyribonucleotide) comprises one or more modified ribonucleotides that have the following structure:

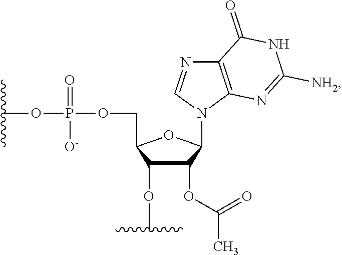

wherein [symbol] indicates the position of attachment to an adjacent ribonucleotide.

In some embodiments, a polynucleotide (e.g., polyribonucleotide) comprises one or more modified ribonucleotides that have the following structure:

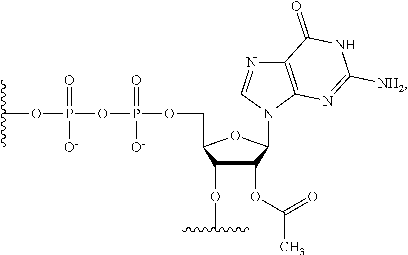

wherein [symbol] indicates the position of attachment to an adjacent ribonucleotide.

In some embodiments, a polynucleotide (e.g., polyribonucleotide) comprises one or more modified ribonucleotides that have the following structure:

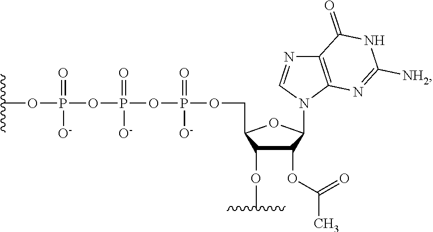

wherein [symbol] indicates the position of attachment to an adjacent ribonucleotide.

In some embodiments, one or more modified ribonucleotides comprises: (i) a 2′-O-acetylated ribose and (ii) a cytosine nucleobase (e.g., cytosine or N4-acetylcytosine). In some embodiments, one or more modified ribonucleotides has a structure provided in Table 6. In some embodiments, one or more modified ribonucleotides has a structure provided in Table 7 when incorporated in a polynucleotide. In some embodiments, a polynucleotide (e.g., a polyribonucleotide) comprises one or more modified ribonucleotides that have a structure provided in Table 7.

In some embodiments, a polynucleotide (e.g., polyribonucleotide) comprises one or more modified ribonucleotides that have the following structure:

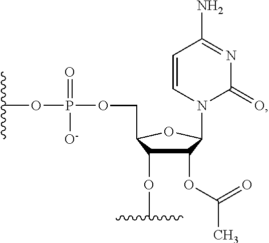

wherein [symbol] indicates the position of attachment to an adjacent ribonucleotide.

In some embodiments, a polynucleotide (e.g., polyribonucleotide) comprises one or more modified ribonucleotides that have the following structure:

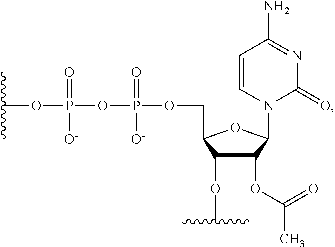

wherein [symbol] indicates the position of attachment to an adjacent ribonucleotide.

In some embodiments, a polynucleotide (e.g., polyribonucleotide) comprises one or more modified ribonucleotides that have the following structure:

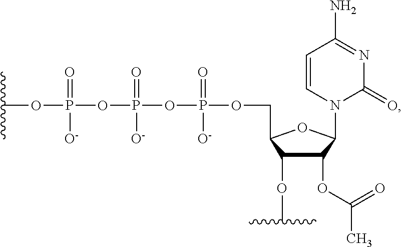

wherein [symbol] indicates the position of attachment to an adjacent ribonucleotide.

In some embodiments, one or more modified ribonucleotides comprising a 2′-O-acetylated ribose comprises a N4-acetylcytosine nucleobase. In some embodiments, one or more modified ribonucleotides has a structure provided in Table 6. In some embodiments, one or more modified ribonucleotides has a structure provided in Table 7 when incorporated in a polynucleotide. In some embodiments, a polynucleotide (e.g., a polyribonucleotide) comprises one or more modified ribonucleotides that have a structure provided in Table 7.

In some embodiments, a polynucleotide (e.g., polyribonucleotide) comprises one or more modified ribonucleotides that have the following structure:

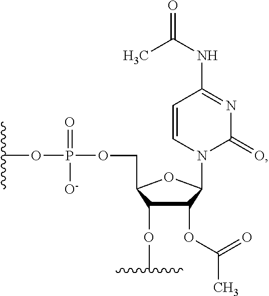

wherein [symbol] Indicates the position of attachment to an adjacent ribonucleotide.

In some embodiments, a polynucleotide (e.g., polyribonucleotide) comprises one or more modified ribonucleotides that have the following structure:

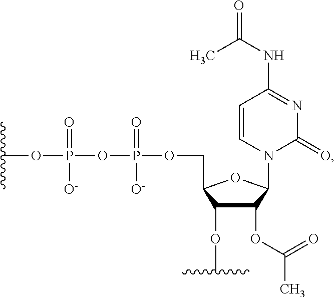

wherein [symbol] indicates the position of attachment to an adjacent ribonucleotide.

In some embodiments, a polynucleotide (e.g., polyribonucleotide) comprises one or more modified ribonucleotides that have the following structure:

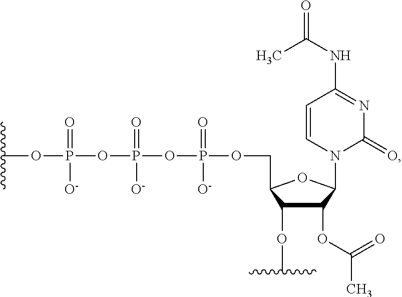

wherein [symbol] indicates the position of attachment to an adjacent ribonucleotide.

In some embodiments, one or more modified ribonucleotides comprising a 2′-O-acetylated ribose comprises a uracil nucleobase (e.g., uracil or 5-hydroxymethyluracil). In some embodiments, one or more modified ribonucleotides has a structure provided in Table 6. In some embodiments, one or more modified ribonucleotides has a structure provided in Table 7 when incorporated in a polynucleotide. In some embodiments, a polynucleotide (e.g., a polyribonucleotide) comprises one or more modified ribonucleotides that have a structure provided in Table 7.

In In some embodiments, a polynucleotide (e.g., polyribonucleotide) comprises one or more modified ribonucleotides that have the following structure:

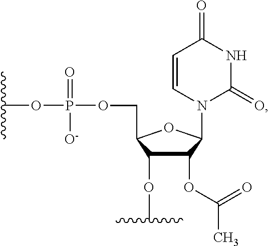

wherein [symbol] indicates the position of attachment to an adjacent ribonucleotide.

In some embodiments, a polynucleotide (e.g., polyribonucleotide) comprises one or more modified ribonucleotides that have the following structure:

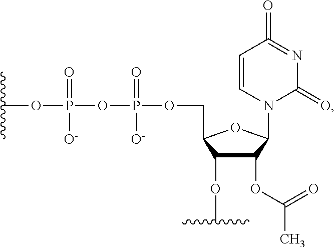

wherein [symbol] indicates the position of attachment to an adjacent ribonucleotide.

In some embodiments, a polynucleotide (e.g., polyribonucleotide) comprises one or more modified ribonucleotides that have the following structure:

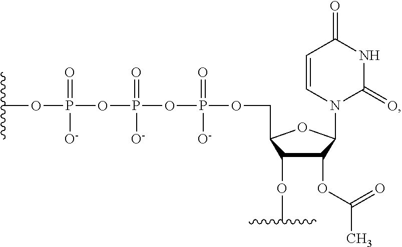

wherein [symbol] indicates the position of attachment to an adjacent ribonucleotide.

In some embodiments, one or more modified ribonucleotides comprising a 2′-O-acetylated ribose comprises a 5-hydroxymethyluracil nucleobase. In some embodiments, one or more modified ribonucleotides has a structure provided in Table 6. In some embodiments, one or more modified ribonucleotides has a structure provided in Table 7 when incorporated in a polynucleotide. In some embodiments, a polynucleotide (e.g., a polyribonucleotide) comprises one or more modified ribonucleotides that have a structure provided in Table 7.

In some embodiments, a polynucleotide (e.g., polyribonucleotide) comprises one or more modified ribonucleotides that have the following structure:

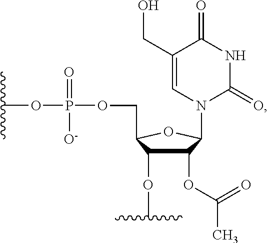

wherein [symbol] indicates the position of attachment to an adjacent ribonucleotide.

In some embodiments, a polynucleotide (e.g., polyribonucleotide) comprises one or more modified ribonucleotides that have the following structure:

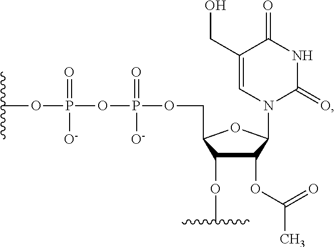

wherein [symbol] indicates the position of attachment to an adjacent ribonucleotide.

In some embodiments, a polynucleotide (e.g., polyribonucleotide) comprises one or more modified ribonucleotides that have the following structure:

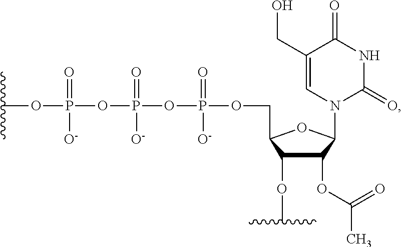

wherein [symbol] indicates the position of attachment to an adjacent ribonucleotide.

In some embodiments, one or more modified ribonucleotides comprising a 2′-O-acetylated ribose comprises a N1-methylpseudouracil nucleobase. In some embodiments, one or more modified ribonucleotides has a structure provided in Table 6. In some embodiments, one or more modified ribonucleotides has a structure provided in Table 7 when incorporated in a polynucleotide. In some embodiments, a polynucleotide (e.g., a polyribonucleotide) comprises one or more modified ribonucleotides that have a structure provided in Table 7.

In some embodiments, a polynucleotide (e.g., polyribonucleotide) comprises one or more modified ribonucleotides that have the following structure:

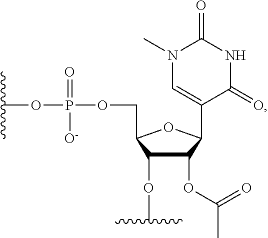

wherein [symbol] indicates the position of attachment to an adjacent ribonucleotide.

In some embodiments, a polynucleotide (e.g., polyribonucleotide) comprises one or more modified ribonucleotides that have the following structure:

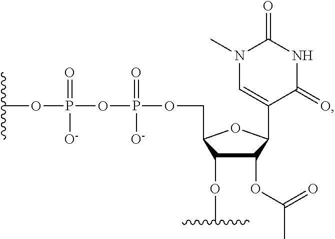

wherein [symbol] indicates the position of attachment to an adjacent ribonucleotide.

In some embodiments, a polynucleotide (e.g., polyribonucleotide) comprises one or more modified ribonucleotides that have the following structure:

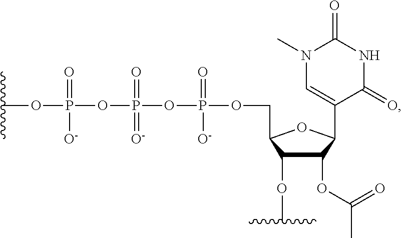

wherein [symbol] indicates the position of attachment to an adjacent ribonucleotide.

In some embodiments of any of the polyribonucleotides disclosed herein, at least 5% of ribose moieties are acetylated (2′-O-acetylated).

In some embodiments of any of the polyribonucleotides disclosed herein, about 5% to about 99% of ribose moieties are acetylated (2′-O-acetylated).

In some embodiments, polyribonucleotide made according to a method disclosed herein comprises a cap structure and a cap structure does not comprise a 2′-O-acetylated ribose.

In some embodiments, polyribonucleotide made according to a method disclosed herein comprises a cap structure and a cap structure comprises a 2′-O-acetylated ribose.

In some embodiments, polyribonucleotide made according to a method disclosed herein further comprises one or more ribonucleotides that does not comprise a 2′-0 acetylated ribose.

In some embodiments, polyribonucleotide made according to a method disclosed herein comprises, comprises a coding region. In some embodiments, a polyribonucleotide is or comprises an RNA oligo; a messenger RNA (mRNA); a gRNA; an inhibitory RNA; an miRNA or siRNA; an antisense oligonucleotide; or any combination thereof.

These, and other aspects encompassed by the present disclosure, are described in more detail below and in the claims.

### BRIEF DESCRIPTION OF THE DRAWINGS

FIG. 1 depicts a schema of exemplary T7, T3, and SP6 promoter proximal sequences in DNA templates which are transcribed to RNA sequences comprising promoter proximal sequences.

FIGS. 2A-2B depict total unmodified RNA yield and purity of RNAs comprising poly-adenine tracts which were made using IVT reactions with a DNA template having poly-adenine tracts at the 3′end of the T7 promoter. FIG. 2A depicts total RNA yield (ug) after IVT reactions of RNAs encoding the FLuc protein bearing increasing poly-adenine tracts (3 adenines, 6 adenines and 12 adenines). FIG. 2B shows an electropherogram generated following analysis of a gel capillary electrophoresis shows the purity of the listed RNAs on FIG. 2A.

FIGS. 3A-3B depict total unmodified RNA yield and purity of RNAs comprising poly-cytosine tracts which were made using IVT reactions with a DNA template having poly-cytosine tracts at the 3′end of the T7 promoter. FIG. 3A depicts total RNA yield (ug) after IVT reactions of RNAs encoding the FLuc protein bearing increasing poly-cytosine tracks (3 cytosines, 6 cytosines and 12 cytosines). FIG. 3B depicts an electropherogram generated following analysis of a gel capillary electrophoresis shows the purity of the listed RNAs on FIG. 3A.

FIGS. 4A-4B depict total unmodified RNA yield and purity of RNAs comprising poly-thymine tracts which were made using IVT reactions with a DNA template having poly-thymine tracts at the 3′end of the T7 promoter. FIG. 4A depicts total RNA yield (ug) after IVT reactions of RNAs encoding the FLuc protein bearing increasing poly-uridine tracks (3 uridines, 6 uridines and 12 uridines). FIG. 4B shows an electropherogram generated following analysis of a gel capillary electrophoresis shows the purity of the listed RNAs on FIG. 4A.

FIGS. 5A-5B depict total unmodified RNA yield and purity of RNAs comprising poly-guanine tracts which were made using IVT reactions with a DNA template having poly-guanine tracts at the 3′end of the T7 promoter. FIG. 5A depicts total RNA yield (ug) after IVT reactions of RNAs encoding the FLuc protein bearing increasing poly-guanine tracks (3 guanines, 6 guanines and 12 guanines). FIG. 5B depicts an electropherogram generated following analysis of a gel capillary electrophoresis shows the purity of the listed RNAs on FIG. 5A.

FIGS. 6A-6B depict total N1-methylpseudouridine modified RNA yield and purity of RNAs comprising poly-adenine tracts which were made using IVT reactions with a DNA template having poly-adenine tracts at the 3′end of the T7 promoter. FIG. 6A depicts total RNA yield (ug) after IVT reactions of RNAs encoding the FLuc protein bearing increasing poly-adenine tracks (3 adenines, 6 adenines and 12 adenines). FIG. 6B depicts an electropherogram generated following analysis of a gel capillary electrophoresis shows the purity of the listed RNAs on FIG. 6A.

FIGS. 7A-7B depict total N1-methylpseudouridine modified RNA yield and purity of RNAs comprising poly-cytosine tracts which were made using IVT reactions with a DNA template having poly-cytosine tracts at the 3′end of the T7 promoter. FIG. 7A depicts total RNA yield (ug) after IVT reactions of RNAs encoding the FLuc protein bearing increasing poly-cytosine tracks (3 cytosines, 6 cytosines and 12 cytosines). FIG. 7B depicts an electropherogram generated following analysis of a gel capillary electrophoresis shows the purity of the listed RNAs on FIG. 7A.

FIGS. 8A-8B depict total N1-methylpseudouridine modified RNA yield and purity of RNAs comprising poly-thymine tracts which were made using IVT reactions with a DNA template having poly-thymine tracts at the 3′end of the T7 promoter. FIG. 8A depicts total RNA yield (ug) after IVT reactions of RNAs encoding the FLuc protein bearing increasing poly-uridine tracks (3 uridines, 6 uridines and 12 uridines). FIG. 8B depicts an electropherogram generated following analysis of a gel capillary electrophoresis shows the purity of the listed RNAs on FIG. 8A.

FIGS. 9A-9B depict total N1-methylpseudouridine modified RNA yield and purity of RNAs comprising poly-guanine tracts which were made using IVT reactions with a DNA template having poly-guanine tracts at the 3′end of the T7 promoter. FIG. 9A depicts total RNA yield (ug) after IVT reactions of RNAs encoding the FLuc protein bearing increasing poly-guanine tracks (3 guanines, 6 guanines and 12 guanines). FIG. 9B depicts an electropherogram generated following analysis of a gel capillary electrophoresis shows the purity of the listed RNAs on FIG. 9A.

FIGS. 10A-10B depict total Ac4C/5hmU modified RNA yield and purity of RNAs comprising poly-adenine tracts which were made using IVT reactions with a DNA template having poly-adenine tracts at the 3′end of the T7 promoter. FIG. 10A depicts total RNA yield (ug) after IVT reactions of RNAs encoding the FLuc protein bearing increasing poly-adenine tracks (3 adenines, 6 adenines and 12 adenines). FIG. 10B depicts an electropherogram generated following analysis of a gel capillary electrophoresis shows the purity of the listed RNAs on FIG. 10A.

FIGS. 11A-11B depict total Ac4C/5hmU modified RNA yield and purity of RNAs comprising poly-cytosine tracts which were made using IVT reactions with a DNA template having poly-cytosine tracts at the 3′end of the T7 promoter. FIG. 11A depicts total RNA yield (ug) after IVT reactions of RNAs encoding the FLuc protein bearing increasing poly-cytosine tracks (3 cytosines, 6 cytosines and 12 cytosines). FIG. 11B depicts an electropherogram generated following analysis of a gel capillary electrophoresis shows the purity of the listed RNAs on FIG. 11A.

FIGS. 12A-12B depict total Ac4C/5hmU modified RNA yield and purity of RNAs comprising poly-thymine tracts which were made using IVT reactions with a DNA template having poly-thymine tracts at the 3′end of the T7 promoter. FIG. 12A depicts total RNA yield (ug) after IVT reactions of RNAs encoding the FLuc protein bearing increasing poly-uridine tracks (3 uridines, 6 uridines and 12 uridines). FIG. 12B depicts an electropherogram generated following analysis of a gel capillary electrophoresis shows the purity of the listed RNAs on FIG. 12A.

FIGS. 13A-13B depicts total Ac4C/5hmU modified RNA yield and purity of RNAs comprising poly-guanine tracts which were made using IVT reactions with a DNA template having poly-guanine tracts at the 3′end of the T7 promoter. FIG. 13A depicts total RNA yield (ug) after IVT reactions of RNAs encoding the FLuc protein bearing increasing poly-guanine tracks (3 guanines, 6 guanines and 12 guanines). FIG. 13B depicts an electropherogram generated following analysis of a gel capillary electrophoresis shows the purity of the listed RNAs on FIG. 13A.

FIG. 14 depicts total 2′O modified RNA yield of RNAs comprising poly-adenine tracts which were made using IVT reactions with a DNA template having poly-adenine tracts at the 3′end of the T7 promoter.

FIGS. 15A-15B depicts total unmodified RNA yield and purity of RNAs comprising poly-adenine tracts which were made using IVT reactions with a DNA template having poly-adenine tracts at the 3′end of the T3 promoter. FIG. 15A depicts total RNA yield (ug) after IVT reactions of RNAs encoding the FLuc protein bearing increasing poly-adenine tracks (3 adenines, 6 adenines and 12 adenines). FIG. 15B depicts an electropherogram generated following analysis of a gel capillary electrophoresis shows the purity of the listed RNAs on FIG. 15A.

FIGS. 16A-16B depict total N1-methylpseudouridine modified RNA yield and purity of RNAs comprising poly-adenine tracts which were made using IVT reactions with a DNA template having poly-adenine tracts at the 3′end of the T3 promoter. FIG. 16A depicts total RNA yield (ug) after IVT reactions of RNAs encoding the FLuc protein bearing increasing poly-adenine tracks (3 adenines, 6 adenines and 12 adenines). FIG. 16B depicts an electropherogram generated following analysis of a gel capillary electrophoresis shows the purity of the listed RNAs on FIG. 16A.

FIGS. 17A-17B depict total Ac4C/5hmU modified RNA yield and purity of RNAs comprising poly-adenine tracts which were made using IVT reactions with a DNA template having poly-adenine tracts at the 3′end of the T3 promoter. FIG. 17A depicts total RNA yield (ug) after IVT reactions of RNAs encoding the FLuc protein bearing increasing poly-adenine tracks (3 adenines, 6 adenines and 12 adenines). FIG. 17B depicts an electropherogram generated following analysis of a gel capillary electrophoresis shows the purity of the listed RNAs on FIG. 17A.

FIGS. 18A-18B depict total unmodified RNA yield and purity of RNAs comprising poly-adenine tracts which were made using IVT reactions with a DNA template having poly-adenine tracts at the 3′end of the SP6 AGA promoter variant. FIG. 18A depicts total RNA yield (ug) after IVT reactions of RNAs encoding the FLuc protein bearing increasing poly-adenine tracks (3 adenines, 6 adenines and 12 adenines). FIG. 18B depicts an electropherogram generated following analysis of a gel capillary electrophoresis shows the purity of the listed RNAs on FIG. 18A.

FIGS. 19A-19B depicts total N1-methylpseudouridine modified RNA yield and purity of RNAs comprising poly-adenine tracts which were made using IVT reactions with a DNA template having poly-adenine tracts at the 3′end of the SP6 AGA promoter variant. FIG. 19A depicts total RNA yield (ug) after IVT reactions of RNAs encoding the FLuc protein bearing increasing poly-adenine tracks (3 adenines, 6 adenines and 12 adenines). FIG. 19B depicts an electropherogram generated following analysis of a gel capillary electrophoresis shows the purity of the listed RNAs on FIG. 19A.

FIGS. 20A-20B depict total Ac4C/5hmU modified RNA yield and purity of RNAs comprising poly-adenine tracts which were made using IVT reactions with a DNA template having poly-adenine tracts at the 3′end of the SP6 AGA promoter variant. FIG. 20A depicts total RNA yield (ug) after IVT reactions of RNAs encoding the FLuc protein bearing increasing poly-adenine tracks (3 adenines, 6 adenines and 12 adenines). FIG. 20B depicts an electropherogram generated following analysis of a gel capillary electrophoresis shows the purity of the listed RNAs on FIG. 20A.

FIGS. 21A-21B depict total unmodified RNA yield and purity of RNAs comprising poly-adenine tracts which were made using IVT reactions with a DNA template having poly-adenine tracts at the 3′end of the SP6 AGG promoter variant. FIG. 21A depicts total RNA yield (ug) after IVT reactions of RNAs encoding the FLuc protein bearing increasing poly-adenine tracks (3 adenines, 6 adenines and 12 adenines). FIG. 21B depicts an electropherogram generated following analysis of a gel capillary electrophoresis shows the purity of the listed RNAs on FIG. 21A.

FIGS. 22A-22B depict total N1-methylpseudouridine modified RNA yield and purity of RNAs comprising poly-adenine tracts which were made using IVT reactions with a DNA template having poly-adenine tracts at the 3′end of the SP6 AGG promoter variant FIG. 22A depicts total RNA yield (ug) after IVT reactions of RNAs encoding the FLuc protein bearing increasing poly-adenine tracks (3 adenines, 6 adenines and 12 adenines). FIG. 22B depicts an electropherogram generated following analysis of a gel capillary electrophoresis shows the purity of the listed RNAs on FIG. 22A.

FIGS. 23A-23B depicts total Ac4C/5hmU modified RNA yield and purity of RNAs comprising poly-adenine tracts which were made using IVT reactions with a DNA template having poly-adenine tracts at the 3′end of the SP6 AGG promoter variant. FIG. 23A depicts total RNA yield (ug) after IVT reactions of RNAs encoding the FLuc protein bearing increasing poly-adenine tracks (3 adenines, 6 adenines and 12 adenines). FIG. 23B depicts an electropherogram generated following analysis of a gel capillary electrophoresis shows the purity of the listed RNAs on FIG. 23A.

FIGS. 24A-24B are graphs showing the effect of RNA transcribed with exemplary DNA templates bearing increasing 5′ poly-adenine tracts after the T7 bacteriophage promoter on cell viability (FIG. 24A) and protein expression (FIG. 24B) in the A549-Dual cell line.

FIGS. 25A-25B depicts characterization of RNAs with poly-adenine tracts which were made using IVT reactions with a DNA template having poly-adenine tracts at the 3′end of the T7 promoter. FIG. 25A depicts effect on cell viability of cells administered RNA having increasing poly-adenine tracts 72 hours after transfection in the A549-Dual cell line. FIG. 25B depicts effect on Luc2 expression in cells administered RNA having increasing 5′ poly-adenine tracts 72 hours after transfection in the A549-Dual cell line.

FIGS. 26A-26B are graphs showing the effect of RNA transcribed with exemplary DNA templates bearing increasing 5′ poly-adenine tracts after the T7 bacteriophage promoter on immunogenicity of generated RNA in the A549-Dual cell line. FIG. 26A shows results from an NF-kB assay, and FIG. 26B shows results from an IRF assay.

FIGS. 27A-27B are graphs showing the immunogenicity of unmodified or modified RNA transcribed from exemplary DNA templates having increasing poly-adenine tracts at the 3′ end of the T7 bacteriophage promoter when delivered to the A549-Dual cell line. FIG. 27A depicts effect on NF-kB activation in cells administered RNAs having increasing 5′ poly-adenine tracts 72 hours after transfection in the A549-Dual cell line. FIG. 27B depicts effect on IRF activation in cells administered RNAs having increasing poly-adenine tracts 72 hours after transfection in the A549-Dual cell line.

### CERTAIN DEFINITIONS

About or approximately: As used herein, the terms “about” and “approximately,” when used herein in reference to a value, refers to a value that is similar, in context to the referenced value. In general, those skilled in the art, familiar with the context, will appreciate the relevant degree of variance encompassed by “about” or “approximately” in that context. For example, in some embodiments, the term “about” or “approximately” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administering: As used herein, the term “administering” or “administration” typically refers to administration of a composition to a subject to achieve delivery of an agent that is, or is included in, the composition. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (e.g., by intratracheal instillation), vaginal, vitreal, etc. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Comparable: As used herein, the term “comparable” refers to two or more agents (e.g., entities or set(s) of conditions), situations, etc., that may not be identical to one another but that are sufficiently similar to permit comparison therebetween so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Delivery/contacting: As used interchangeably herein, the term “delivery,” “delivering,” or “contacting” refers to introduction of a polynucleotide (e.g., as described herein) or a fusion polypeptide (e.g., as described herein) into a target cell. A target cell can be cultured in vitro or ex vivo or be present in a subject (in vivo). Methods of introducing a polynucleotide (e.g., as described herein) or a fusion polypeptide (e.g., as described herein) into a target cell can vary with in vitro, ex vivo, or in vivo applications. In some embodiments, a polynucleotide (e.g., as described herein) or a fusion polypeptide (e.g., as described herein) can be introduced into a target cell in a cell culture by in vitro transfection. In some embodiments, a polynucleotide (e.g., as described herein) or a fusion polypeptide (e.g., as described herein) can be introduced into a target cell via delivery vehicles (e.g., nanoparticles, liposomes, and/or complexation with a cell-penetrating agent). In some embodiments, a polynucleotide (e.g., as described herein) or a fusion polypeptide (e.g., as described herein) can be introduced into a target cell in a subject by administering a polynucleotide (e.g., as described herein) or a fusion polypeptide (e.g., as described herein) to a subject.

Encode: As used herein, the team “encode” or “encoding” refers to a first molecule that is produced by a second molecule, wherein the sequence information of the second molecule determines the sequence of the first molecule. For example, a first molecule can have a defined sequence of nucleotides (e.g., a polyribonucleotide) or a defined sequence of amino acids which are determined by the sequence of the second molecule (e.g., a polynucleotide). For example, a DNA molecule (e.g., a second molecule) can encode an RNA molecule (e.g., a second molecule, for example by a transcription process that includes a DNA-dependent RNA polymerase enzyme) or a polypeptide (e.g., a first molecule for example by a transcription and a translation process). An RNA molecule (e.g., a second molecule) can encode a polypeptide (e.g., a first molecule for example by a translation process). Thus, a gene, a cDNA, or an RNA molecule encodes a polypeptide if transcription and translation of RNA corresponding to that gene produces the polypeptide in a cell or other biological system.

Functional: As used herein, the term “functional” is used to refer to a form or fragment of an entity that exhibits a particular property and/or activity.

Fragment: A “fragment” of a material or entity as described herein has a structure that includes a discrete portion of the whole, but lacks one or more moieties found in the whole. In some embodiments, a fragment consists of such a discrete portion. In some embodiments, a fragment consists of or comprises a characteristic structural element or moiety found in the whole. In some embodiments, a fragment comprises a polynucleotide fragment. In some embodiments, a fragment comprises a polypeptide fragment. In some embodiments, a polynucleotide fragment or a polypeptide fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) as found in the whole polynucleotide or whole polypeptide. In some embodiments, a polynucleotide fragment or a polypeptide fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units (e.g., residues) found in the whole polynucleotide or whole polypeptide. The whole polypeptide or whole polynucleotide may in some embodiments be referred to as the “parent” of the polynucleotide fragment or polypeptide fragment.

Nucleic acid/Oligonucleotide/Polynucleotide: As used herein, the terms “nucleic acid” and “polynucleotide” and “oligonucleotide” are used interchangeably, and refer to a polymer of 3 nucleotides or more. In some embodiments, a nucleic acid comprises DNA. In some embodiments, a nucleic acid comprises RNA. In some embodiments, a nucleic acid comprises messenger RNA (mRNA). In some embodiments, a nucleic acid is single stranded. In some embodiments, a nucleic acid is double stranded. In some embodiments, a nucleic acid comprises both single and double stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid”. In some embodiments, a nucleic acid comprises one or more, or all, natural nucleosides (e.g., adenosine, cytidine, guanosine, uridine, thymidine, deoxyadenosine, deoxycytidine, deoxyguanosine, deoxythymidine, deoxyuridine). In some embodiments, a nucleic acid comprises one or more, or all, non-natural nucleotides. In some embodiments, a non-natural nucleoside comprises a nucleoside analog (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 6-O-methylguanine, 2-thiocytidine, a nucleoside comprising a methylated base, a nucleoside comprising an intercalated base, and combinations thereof). In some embodiments, a non-natural nucleoside comprises a modified nucleoside, e.g., as described herein. In some embodiments, a non-natural nucleoside comprises N4-acetylcytidine, 5-hydroxymethyluridine and/or N1-methylpseudouridine. In some embodiments, a non-natural nucleoside comprises one or more modified sugars (e.g., 2′-fluororibose, ribose, 2′-deoxyribose, arabinose, and hexose) as compared to those in natural nucleosides. In some embodiments, a non-natural nucleoside comprises a 2′-O-acetylated ribose. As used herein, “adenosine nucleosides” encompasses a natural adenosine nucleoside, as well as modified adenosine nucleosides. Accordingly, when a percentage of adenosine nucleosides is provided herein, the percentage is based on a total number of natural and modified adenosine nucleosides. Similarly, “cytidine nucleosides” encompasses a natural cytidine nucleoside, as well as modified cytidine nucleosides (e.g., N4-acetylcytidine or 2′-O-acetylated N4-acetylcytidine); “guanosine nucleosides” encompasses a natural guanosine nucleoside, as well as modified guanosine nucleosides; and “uridine nucleosides” encompasses a natural uridine nucleoside, as well as modified uridine nucleosides (e.g., 5-hydroxymethyluridine or 2′-O-acetylated 5-hydroxymethyluridine). Further, as used herein, “adenine nucleobase” encompasses a natural adenine nucleobase, as well as modified adenine nucleobase; “cytosine nucleobase” encompasses a natural cytosine nucleobase, as well as modified cytosine nucleobase (e.g., N4-acetylcytosine); “guanine nucleobase” encompasses a natural guanine nucleobase, as well as modified guanine nucleobase; and “uracil nucleobase” encompasses a natural uracil nucleobase, as well as modified uracil nucleobase (e.g., 5-hydroxymethyluracil). In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro, reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, or 20,000 nucleotides long. When a number of nucleotides is used as an indication of size, e.g., of a polynucleotide, a certain number of nucleotides refers to the number of nucleotides on a single strand, e.g., of a polynucleotide.

Polypeptide: The term “polypeptide”, as used herein, generally has its art-recognized meaning of a polymer of at least three amino acids or more. Those of ordinary skill in the art will appreciate that the term “polypeptide” is intended to be sufficiently general as to encompass not only polypeptides having a complete sequence recited herein, but also to encompass polypeptides that represent functional, biologically active, or characteristic fragments, portions or domains (e.g., fragments, portions, or domains retaining at least one activity) of such complete polypeptides. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, polypeptides may comprise natural amino acids, non-natural amino acids, synthetic amino acids, and combinations thereof.

RNA oligonucleotide: As used herein, the term “RNA oligonucleotide” refers to an oligonucleotide of ribonucleotides. In some embodiments, an RNA oligonucleotide is single stranded. In some embodiments, an RNA oligonucleotide is double stranded. In some embodiments, an RNA oligonucleotide comprises both single and double stranded portions. In some embodiments, an RNA oligonucleotide can comprise a backbone structure as described in the definition of “Nucleic acid/Oligonucleotide” above. An RNA oligonucleotide can be a regulatory RNA (e.g., siRNA, microRNA, etc.), or a messenger RNA (mRNA) oligonucleotide. In some embodiments an RNA oligonucleotide can comprise at its 3′ end a poly(A) region. In some embodiments an RNA oligonucleotide comprises at its 5′ end a cap structure, e.g., for recognizing and attachment of an RNA to a ribosome to initiate translation. In some embodiments, a polynucleotide comprises an RNA oligonucleotide. When a number of ribonucleotides is used as an indication of size, e.g., of a polynucleotide, a certain number of nucleotides refers to the number of ribonucleotides on a single strand.

Subject: As used herein, the term “subject” refers an organism, typically a mammal (e.g., a human). In some embodiments, a subject is suffering from a disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Variant: As used herein, the term “variant” refers to an entity that shows significant structural identity with a reference entity but differs structurally from the reference entity in the presence or level of one or more chemical moieties as compared with the reference entity. In many embodiments, a variant also differs functionally from its reference entity. In general, whether a particular entity is properly considered to be a “variant” of a reference entity is based on its degree of structural identity with the reference entity. For example, a variant polypeptide may differ from a reference polypeptide as a result of one or more differences in amino acid sequence and/or one or more differences in chemical moieties (e.g., carbohydrates, lipids, etc.) covalently attached to the polypeptide backbone. Alternatively or additionally, in some embodiments, a variant polypeptide does not share at least one characteristic sequence element with a reference polypeptide. In some embodiments, the reference polypeptide has one or more biological activities. In some embodiments, a variant polypeptide shares one or more of the biological activities of the reference polypeptide. In some embodiments, a variant polypeptide lacks one or more of the biological activities of the reference polypeptide. In some embodiments, a variant polypeptide shows a reduced level of one or more biological activities as compared with the reference polypeptide.

Standard techniques may be used for recombinant DNA, oligonucleotide synthesis, e.g., RNA synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Green and Sambrook, Molecular Cloning: A Laboratory Manual (4^{th} ed., Cold Spring Harbor Laboratory Press, Cold Spring Harbor, N.Y. (2012)), which is incorporated herein by reference for any purpose.

### DETAILED DESCRIPTION OF CERTAIN EMBODIMENTS

RNA-based gene therapies have emerged as a promising therapeutic modality, e.g., due to their ability to manipulate gene expression and/or produce therapeutic proteins, offering the potential to target a wide range of diseases. However, the potential of RNA-based technologies as a therapeutic intervention is hampered by challenges in RNA manufacturing. For example, producing RNAs, e.g., therapeutic RNAs, at a purity and/or yield to be useful for therapeutic applications has been particularly challenging. Often times increasing RNA yield sacrifices RNA purity, or vice versa. Additionally, current methodologies for RNA manufacturing to produce RNA at sufficient purity and/or yield for therapeutics applications is very costly, thus hampering the development of RNAs as therapeutics.

RNA manufacturing is an essential area of RNA therapeutic development particularly for advancing the market for RNA therapeutics. A recent analysis estimated a total market size of RNA therapeutics in 2035 of $7-10 billion in the US. This analysis focused on prophylactic RNA vaccines, without considering therapeutic vaccines and other therapeutics for treating non-infectious diseases (Xie, W., et. al., 2021). By being able to increase RNA yield while maintaining its purity would enable cost-effective manufacturing, improved RNA quality and repeated dosing, as well as a rapidly increase access to this technology which has immense potential.

Traditionally, the T7 bacteriophage RNA polymerase (T7 RNAP) system has been widely used for RNA synthesis due to characteristics such as: a monomeric protein structure, high RNA yield, high processivity of long RNA transcripts, and high specificity for its promoter sequence (Sari Y. et. al., 2024). A prior report demonstrated that an engineered T7 RNA polymerase (RNAP) variant having a G47A/884G mutation reduced double-stranded RNA (dsRNA). However, the evaluation of RNA yield revealed a global decrease in RNA yield across transcripts of different lengths by the utilization of the double T7 RNAP mutant compared to the WT T7 RNAP (Dousis, A. et. al., 2022; Obexer, R. & Lovelock, S. L., 2023). Thus, the double T7 RNAP mutant is not beneficial in producing RNA with increased purity and yield, e.g., as required for use in therapeutic applications.

It has been disclosed that downstream sequences from position +1 to +6 of the T7 promoter sequence could influence RNA yield (Milligan, J., et. al., 1987). Yet another report, created a consensus T7 promoter (T7Max) based on previous T7 promoter variants, and reported an increase of only 0.5-fold by the use of the T7 Max promoter on the DNA IVT input compared to the WT T7 promoter (Deich C., et. al., 2023). However, this report is silent regarding the effect of T7 variants on RNA purity, an important feature in manufacturing RNA for therapeutic applications.

In contrast with the T7 bacteriophage promoter, the SP6 and T3 bacteriophage promoters have received less attention. This is despite the high degree of protein homology between the T7, SP6 and T3 promoter sequences. Accordingly, the benefit of altering and/or improving the SP6 and T3 promoters for RNA manufacturing is unclear. For SP6 and T3, there are reports describing the activity and sequence recognition of these promoters in vitro in comparison to the T7 promoter (Padmanabhan, R., et. al., 2020). However, engineering of the SP6 and/or T3 promoters for use in RNA manufacturing has not been reported.

Among other things, the present disclosure provides certain insights and technologies relating to improved polynucleotides for use in manufacturing RNA with sufficient yield and/or purity for use in downstream applications such as therapeutics. In particular, provided herein is the insight that polynucleotides comprising a promoter and a promoter proximal sequence comprising at least two consecutive identical nucleotides (e.g., poly-adenine, poly-cytosine and/or poly-thymine tracts) can enhance the yield and/or purity of polyribonucleotides produced with these polynucleotides. Specifically, polynucleotides provided herein can be used for producing polyribonucleotides with high yield while maintaining the purity of the polyribonucleotides. As shown in Examples 2-5 polynucleotides provided herein can be used to increase polyribonucleotide yield by about 2-fold to 5-fold. The increase polyribonucleotide yield is observed with polyribonucleotides comprising unmodified nucleotides or polyribonucleotides comprising modified nucleotides.

This disclosure further recognizes that the increase in yield and/or purity observed with polyribonucleotides made from polynucleotides having a promoter and a promoter proximal sequence comprising at least two consecutive identical nucleotides (e.g., poly-adenine, poly-cytosine and/or poly-thymine tracts) is not due to the mere length of the promoter proximal sequence. For example, as shown in Example 3, when polynucleotides having poly-guanine tracts at the 3′ end of the promoter sequence were used to synthesize polyribonucleotides, the yield and purity of the resulting polyribonucleotides was significantly reduced compared to control polyribonucleotides synthesized with polynucleotides having a similar promoter without a poly-guanine tract at the 3′ end of the promoter. This data shows that the beneficial effects in yield and purity observed with polyribonucleotides made from polynucleotides having a promoter and a promoter proximal sequence comprising at least two consecutive identical nucleotides (e.g., poly-adenine, poly-cytosine and/or poly-thymine tracts) cannot be solely attributed to the length of the promoter proximal sequence itself.

Furthermore, polyribonucleotides made from polynucleotides disclosed herein can be successfully transfected into cells without a change in cell viability. See Example 6 showing no change in cell viability from transfection of polyribonucleotides made with polynucleotides disclosed herein compared to cell viability of cells transfected with polyribonucleotides made with comparable polynucleotides without promoter proximal sequences. Polyribonucleotides made from polynucleotides disclosed herein also result in expression of a target (e.g., a target polypeptide) at a comparable level as compared to target expression from polyribonucleotides made with otherwise similar polynucleotides without promoter proximal sequences (see Example 5.

Additionally, when introduced in vivo, polyribonucleotides made from polynucleotides disclosed herein showed reduced immunogenicity in particular when the polyribonucleotides include modified nucleotides (such as N4-acetylcytidine and/or 5-hydroxymethyluridine) or 2′O ribose modifications (see Example 7). Thus, polyribonucleotides made from polynucleotides disclosed herein are not only produced at higher yield and/or purity compared to polyribonucleotides made from polynucleotides without promoter proximal sequences, but also have meaningful characteristics that support their utility in various applications.

A person of skill in the art will appreciate upon reading this disclosure that the present disclosure is the first to recognize that polynucleotides disclosed herein which include a promoter sequence, a promoter proximal sequence comprising two or more consecutive nucleotides, and a sequence encoding a target (e.g., a payload) can be beneficial in producing polyribonucleotides with sufficient yield and/or purity required for downstream applications such as therapeutic uses. Also provided herein is the insight that polyribonucleotide made with polynucleotides disclosed herein can be safely administered as a therapeutic as observed by the similar expression profile, viability and immunogenicity as compared to otherwise similar polyribonucleotides made with polynucleotides without promoter proximal sequences as disclosed herein.

The present disclosure provides, among other things, polynucleotides disclosed herein and compositions comprising the same, methods of using polynucleotides disclosed herein to make polyribonucleotides with a desired yield and/or purity (e.g., a yield and/or purity sufficient for therapeutic applications), as well as polyribonucleotides made with the disclosed methods, and compositions comprising the same. Further disclosed herein are uses (e.g., applications such as therapeutic applications) of polyribonucleotides made with the disclosed methods.

### Polynucleotides Comprising Exemplary Promoters and a Promoter Proximal Sequences

Provided herein are polynucleotide sequences comprising a promoter sequence, a promoter proximal sequence comprising two or more consecutive nucleotides, and a sequence encoding a target (e.g., a payload). In polynucleotides disclosed herein, promoter proximal sequences are positioned at the 3′ end of a promoter sequence (e.g., downstream of the promoter sequence), and at the 5′ end of a sequence encoding a target (e.g., at the 5′ end of a sequence encoding a target).

Among other things, the present disclosure provides polynucleotide sequences comprising exemplary promoter sequences disclosed herein. Promoters that can be useful in polynucleotides disclosed herein include promoters recognized by RNA polymerases for generating in vitro transcribed polyribonucleotides. Exemplary promoters recognized by RNA polymerases include T7, T3 or SP6 promoters.

In some embodiments, a promoter sequence is a T7 bacteriophage promoter or a variant or a fragment thereof.

A person of skill in the art will recognize T7 bacteriophage RNA polymerase (T7 RNAP) systems may be used for RNA synthesis due to desirable characteristics (e.g., a monomeric protein structure, high RNA yield, processivity of long RNA transcripts, high specificity for its promoter sequence). See, for example, Sari, Y., “Comprehensive evaluation of T7 promoter for enhanced yield and quality in mRNA production”, Scientific Reports, 2024 14(1), 9655, which is incorporated herein by reference in its entirety.

In some embodiments, a T7 promoter comprises an AGG sequence at the 3′ end of the promoter sequence. In some embodiments, a T7 promoter does not comprise an AGG sequence at the 3′ end of the promoter sequence. In some embodiments, a T7 promoter comprises a sequence of SEQ ID NO: 6.

A person of skill will appreciate upon reading this disclosure that T3 and SP6 promoters comprise a series of conserved sequence elements compared with a wild type T7 promoter (e.g., transcription start site sequence element, motif and unwinding region sequence element, core region promoter sequence element). See, for example, Padmanabhan, R., Sarcar, S. N., & Miller, D. L. (2020). Promoter length affects the initiation of T7 RNA polymerase in vitro: New insights into promoter/polymerase co-evolution. Journal of Molecular Evolution, 88(2), 179-193, which is incorporated herein by reference in its entirety.

In some embodiments, some embodiments, a promoter sequence is a T3 bacteriophage promoter or a variant or a fragment thereof. In some embodiments, a T3 promoter comprises a sequence of SEQ ID NO: 17.

In some embodiments, a promoter sequence is an SP6 promoter or a variant or a fragment thereof.

In some embodiments, an SP6 promoter comprises an AGG sequence at the 3′ end of the promoter sequence (e.g., before a promoter proximal sequence). In some embodiments, an SP6 promoter does not comprise an AGG sequence at the 3′ end of the promoter sequence. In some embodiments, an SP6 promoter comprises a sequence of SEQ ID NO: 27.

In some embodiments, an SP6 promoter comprises an AGA sequence at the 3′ end of the promoter sequence (e.g., before a promoter proximal sequence). In some embodiments, an SP6 promoter does not comprise an AGA sequence at the 3′ end of the promoter sequence. In some embodiments, an SP6 promoter comprises a sequence of SEQ ID NO: 60.

As disclosed herein, promoter sequences useful in polynucleotides disclosed herein include promoters recognized by RNA polymerase. Such promoters include a transcription start site which is typically positioned at or near the 3′ end of a promoter sequence and upstream of a sequence encoding a target. In polynucleotides disclosed herein, a transcription start site is positioned in a promoter sequence.

In some embodiments of polynucleotides disclosed herein, a transcription start site is positioned in a T7 promoter sequence. In some embodiments, a transcription start site is within SEQ ID NO: 6. In some embodiments, when a polynucleotide comprising a T7 promoter sequence and a promoter proximal sequence is used to make a polyribonucleotide, a promoter proximal sequence is transcribed into the polyribonucleotide sequence such that the polyribonucleotide sequence comprises a promoter proximal sequence and a sequence encoding a target.

In some embodiments of polynucleotides disclosed herein, a transcription start site is positioned in a T7 promoter sequence. In some embodiments, a transcription start site is within SEQ ID NO: 70. In some embodiments, when a polynucleotide comprising a T7 promoter sequence and a promoter proximal sequence is used to make a polyribonucleotide, a promoter proximal sequence is transcribed into the polyribonucleotide sequence such that the polyribonucleotide sequence comprises a promoter proximal sequence and a sequence encoding a target.

In some embodiments of polynucleotides disclosed herein, a transcription start site is positioned in a T3 promoter sequence. In some embodiments, a transcription start site is within SEQ ID NO: 17. In some embodiments, when a polynucleotide comprising a T3 promoter sequence and a promoter proximal sequence is used to make a polyribonucleotide, a promoter proximal sequence is transcribed into the polyribonucleotide sequence such that the polyribonucleotide sequence comprises a promoter proximal sequence and a sequence encoding a target.

In some embodiments of polynucleotides disclosed herein, a transcription start site is positioned in a T3 promoter sequence. In some embodiments, a transcription start site is within SEQ ID NO: 81. In some embodiments, when a polynucleotide comprising a T3 promoter sequence and a promoter proximal sequence is used to make a polyribonucleotide, a promoter proximal sequence is transcribed into the polyribonucleotide sequence such that the polyribonucleotide sequence comprises a promoter proximal sequence and a sequence encoding a target.

In some embodiments of polynucleotides disclosed herein, a transcription start site is positioned in a SP6 promoter sequence. In some embodiments, a transcription start site is within SEQ ID NO: 27. In some embodiments, when a polynucleotide comprising a SP6 promoter sequence and a promoter proximal sequence is used to make a polyribonucleotide, a promoter proximal sequence is transcribed into the polyribonucleotide sequence such that the polyribonucleotide sequence comprises a promoter proximal sequence and a sequence encoding a target.

In some embodiments of polynucleotides disclosed herein, a transcription start site is positioned in a SP6 promoter sequence. In some embodiments, a transcription start site is within SEQ ID NO: 60. In some embodiments, when a polynucleotide comprising a SP6 promoter sequence and a promoter proximal sequence is used to make a polyribonucleotide, a promoter proximal sequence is transcribed into the polyribonucleotide sequence such that the polyribonucleotide sequence comprises a promoter proximal sequence and a sequence encoding a target.

### Promoter Proximal Sequences

This disclosure provides polynucleotide sequences comprising a promoter sequence comprising a 5′ end and a 3′ end (e.g., as disclosed herein) and a promoter proximal sequence adjacent to the 3′ end of the promoter sequence (e.g., as disclosed herein). In some embodiments, a promoter proximal sequence comprises two or more consecutive nucleotides.

In some embodiments, a promoter proximal sequence comprises adenines, thymines, cytosines, or any combination thereof. In some embodiments, a promoter proximal sequence comprises two or more consecutive adenines, thymines, or cytosines. In some embodiments, a promoter proximal sequence comprises two or more consecutive adenines. In some embodiments, a promoter proximal sequence comprises two or more consecutive cytosines. In some embodiments, a promoter proximal sequence comprises two or more consecutive thymines.

In some embodiments, a promoter proximal sequence is at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides in length.

In some embodiments, a promoter proximal sequence is no more than about 500 nucleotides in length.

In some embodiments, a promoter proximal sequence is about 3 nucleotides to about 500, about 3 to about 400, about 3 to about 300, about 3 to about 200, about 3 to about 100, about 3 to about 50, about 3 to about 40, about 3 to about 30, about 3 to about 20, about 3 to about 15, about 3 to about 14, about 3 to about 13, about 3 to about 12, to about 11, about 3 to about 10, about 3 to about 9, about 3 to about 8, about 3 to about 7, about 3 to about 6, about 3 to about 5 nucleotides in length.

In some embodiments, a promoter proximal sequence disclosed herein is about 4 to about 500, about 5 to about 500, about 10 to about 500, about 20 to about 500, about 50 to about 500, about 100 to about 500, about 200 to about 500, about 300 to about 500 or about 400 to about 500 nucleotides in length.

In some embodiments, a promoter proximal sequence disclosed herein is at least about 3 nucleotides, at least about 4 nucleotides, at least about 5 nucleotides, at least about 6 nucleotides, at least about 7 nucleotides, at least about 8 nucleotides, at least about 9 nucleotides, at least about 10 nucleotides, at least about 11 nucleotides, at least about 12 nucleotides, at least about 13 nucleotides, at least about 14 nucleotides, at least about 15 nucleotides, at least about 16 nucleotides, at least about 17 nucleotides, at least about 18 nucleotides, at least about 19 nucleotides, or at least about 20 nucleotides in length.

In some embodiments, a promoter proximal sequence described herein comprises two or more consecutive nucleotides that are the same nucleotide.

In some embodiments, a promoter proximal sequence described herein comprises two or more consecutive adenines. In some embodiments, a promoter proximal sequence comprises adenines. In some embodiments, a promoter proximal sequence described herein comprises at least 2 adenines, at least 3 adenines, at least 4 adenines, at least 5 adenines, at least 6 adenines, at least 7 adenines, at least 8 adenines, at least 9 adenines, at least 10 adenines, at least 11 adenines, at least 12 adenines, at least 13 adenines, at least 14 adenines, at least 15 adenines, at least 16 adenines, at least 17 adenines, at least 18 adenines, at least 19 adenines, at least 20 adenines.

In some embodiments, a promoter proximal described herein comprises about 2 to about 20 adenines, about 3 to about 20 adenines, about 4 to about 20 adenines, about 5 to about 20 adenines, about 6 to about 20 adenines, about 7 to about 20 adenines, about 8 to about 20 adenines, about 9 to about 20 adenines, about 10 to about 20 adenines, about 11 to about 20 adenines, about 12 to about 20 adenines, about 13 to about 20 adenines, about 14 to about 20 adenines, about 15 to about 20 adenines, about 16 to about 20 adenines, about 17 to about 20 adenines, about 18 to about 20 adenines, about 19 to about 20 adenines, about 2 to about 19 adenines, about 2 to about 18 adenines, about 2 to about 17 adenines, about 2 to about 16 adenines, about 2 to about 15 adenines, about 2 to about 14 adenines, about 2 to about 13 adenines, about 2 to about 12 adenines, about 2 to about 11 adenines, about 2 to about 10 adenines, about 2 to about 9 adenines, about 2 to about 8 adenines, about 2 to about 7 adenines, about 2 to about 6 adenines, about 2 to about 5 adenines, about 2 to about 4 adenines, about 2 to about 3 adenines.

In some embodiments, a polynucleotide disclosed herein comprises a promoter sequence (e.g., T7, SP3, or SP6 as disclosed herein) and a promoter proximal sequence comprising at least 2 adenines, at least 3 adenines, at least 4 adenines, at least 5 adenines, at least 6 adenines, at least 7 adenines, at least 8 adenines, at least 9 adenines, at least 10 adenines, at least 11 adenines, at least 12 adenines, at least 13 adenines, at least 14 adenines, at least 15 adenines, at least 16 adenines, at least 17 adenines, at least 18 adenines, at least 19 adenines, at least 20 adenines.

In some embodiments, a polynucleotide disclosed herein comprises a sequence provided in any one of Tables 1-3. In some embodiments, a polynucleotide herein comprises a sequence of SEQ ID NO: 7. In some embodiments, a polynucleotide herein comprises a sequence of SEQ ID NO: 8. In some embodiments, a polynucleotide herein comprises a sequence of SEQ ID NO: 9. In some embodiments, a polynucleotide herein comprises a sequence of SEQ ID NO: 10. In some embodiments, a polynucleotide herein comprises a sequence of SEQ ID NO: 71. In some embodiments, a polynucleotide herein comprises a sequence of SEQ ID NO: 72. In some embodiments, a polynucleotide herein comprises a sequence of SEQ ID NO: 73. In some embodiments, a polynucleotide herein comprises a sequence of SEQ ID NO: 74. In some embodiments, a polynucleotide herein comprises a sequence of SEQ ID NO: 18. In some embodiments, a polynucleotide herein comprises a sequence of SEQ ID NO: 19. In some embodiments, a polynucleotide herein comprises a sequence of SEQ ID NO: 20. In some embodiments, a polynucleotide herein comprises a sequence of SEQ ID NO: 82. In some embodiments, a polynucleotide herein comprises a sequence of SEQ ID NO: 83. In some embodiments, a polynucleotide herein comprises a sequence of SEQ ID NO: 84. In some embodiments, a polynucleotide herein comprises a sequence of SEQ ID NO: 28. In some embodiments, a polynucleotide herein comprises a sequence of SEQ ID NO: 29. In some embodiments, a polynucleotide herein comprises a sequence of SEQ ID NO: 30. In some embodiments, a polynucleotide herein comprises a sequence of SEQ ID NO: 61. In some embodiments, a polynucleotide herein comprises a sequence of SEQ ID NO: 62. In some embodiments, a polynucleotide herein comprises a sequence of SEQ ID NO: 63.

In some embodiments, a promoter proximal sequence described herein comprises two or more consecutive cytosines. In some embodiments, a promoter proximal sequence described herein comprises at least 2 cytosines, at least 3 cytosines, at least 4 cytosines, at least 5 cytosines, at least 6 cytosines, at least 7 cytosines, at least 8 cytosines, at least 9 cytosines, at least 10 cytosines, at least 11 cytosines, at least 12 cytosines, at least 13 cytosines, at least 14 cytosines, at least 15 cytosines, at least 16 cytosines, at least 17 cytosines, at least 18 cytosines, at least 19 cytosines, at least 20 cytosines.

In some embodiments, a promoter proximal described herein comprises about 2 to about 20 cytosines, about 3 to about 20 cytosines, about 4 to about 20 cytosines, about 5 to about 20 cytosines, about 6 to about 20 cytosines, about 7 to about 20 cytosines, about 8 to about 20 cytosines, about 9 to about 20 cytosines, about 10 to about 20 cytosines, about 11 to about 20 cytosines, about 12 to about 20 cytosines, about 13 to about 20 cytosines, about 14 to about 20 cytosines, about 15 to about 20 cytosines, about 16 to about 20 cytosines, about 17 to about 20 cytosines, about 18 to about 20 cytosines, about 19 to about 20 cytosines, about 2 to about 19 cytosines, about 2 to about 18 cytosines, about 2 to about 17 cytosines, about 2 to about 16 cytosines, about 2 to about 15 cytosines, about 2 to about 14 cytosines, about 2 to about 13 cytosines, about 2 to about 12 cytosines, about 2 to about 11 cytosines, about 2 to about 10 cytosines, about 2 to about 9 cytosines, about 2 to about 8 cytosines, about 2 to about 7 cytosines, about 2 to about 6 cytosines, about 2 to about 5 cytosines, about 2 to about 4 cytosines, about 2 to about 3 cytosines.

In some embodiments, a polynucleotide disclosed herein comprises a promoter sequence (e.g., T7, SP3, or SP6 as disclosed herein) and a promoter proximal sequence comprising at least 2 cytosines, at least 3 cytosines, at least 4 cytosines, at least 5 cytosines, at least 6 cytosines, at least 7 cytosines, at least 8 cytosines, at least 9 cytosines, at least 10 cytosines, at least 11 cytosines, at least 12 cytosines, at least 13 cytosines, at least 14 cytosines, at least 15 cytosines, at least 16 cytosines, at least 17 cytosines, at least 18 cytosines, at least 19 cytosines, at least 20 cytosines.

In some embodiments, a polynucleotide disclosed herein comprises a sequence provided in any one of Tables 1-3. In some embodiments, a polynucleotide herein comprises a sequence of SEQ ID NO: 14. In some embodiments, a polynucleotide herein comprises a sequence of SEQ ID NO: 15. In some embodiments, a polynucleotide herein comprises a sequence of SEQ ID NO: 16. In some embodiments, a polynucleotide herein comprises a sequence of SEQ ID NO: 78. In some embodiments, a polynucleotide herein comprises a sequence of SEQ ID NO: 79. In some embodiments, a polynucleotide herein comprises a sequence of SEQ ID NO: 80. In some embodiments, a polynucleotide herein comprises a sequence of SEQ ID NO: 24. In some embodiments, a polynucleotide herein comprises a sequence of SEQ ID NO: 25. In some embodiments, a polynucleotide herein comprises a sequence of SEQ ID NO: 26. In some embodiments, a polynucleotide herein comprises a sequence of SEQ ID NO: 88. In some embodiments, a polynucleotide herein comprises a sequence of SEQ ID NO: 89. In some embodiments, a polynucleotide herein comprises a sequence of SEQ ID NO: 90. In some embodiments, a polynucleotide herein comprises a sequence of SEQ ID NO: 34. In some embodiments, a polynucleotide herein comprises a sequence of SEQ ID NO: 35. In some embodiments, a polynucleotide herein comprises a sequence of SEQ ID NO: 36.

In some embodiments, a promoter proximal sequence described herein comprises two or more consecutive thymines. In some embodiments, a promoter proximal sequence comprises thymines. In some embodiments, a promoter proximal sequence described herein comprises at least 2 thymines, at least 3 thymines, at least 4 thymines, at least 5 thymines, at least 6 thymines, at least 7 thymines, at least 8 thymines, at least 9 thymines, at least 10 thymines, at least 11 thymines, at least 12 thymines, at least 13 thymines, at least 14 thymines, at least 15 thymines, at least 16 thymines, at least 17 thymines, at least 18 thymines, at least 19 thymines, at least 20 thymines.

In some embodiments, a promoter proximal described herein comprises about 2 to about 20 thymines, about 3 to about 20 thymines, about 4 to about 20 thymines, about 5 to about 20 thymines, about 6 to about 20 thymines, about 7 to about 20 thymines, about 8 to about 20 thymines, about 9 to about 20 thymines, about 10 to about 20 thymines, about 11 to about 20 thymines, about 12 to about 20 thymines, about 13 to about 20 thymines, about 14 to about 20 thymines, about 15 to about 20 thymines, about 16 to about 20 thymines, about 17 to about 20 thymines, about 18 to about 20 thymines, about 19 to about 20 thymines, about 2 to about 19 thymines, about 2 to about 18 thymines, about 2 to about 17 thymines, about 2 to about 16 thymines, about 2 to about 15 thymines, about 2 to about 14 thymines, about 2 to about 13 thymines, about 2 to about 12 thymines, about 2 to about 11 thymines, about 2 to about 10 thymines, about 2 to about 9 thymines, about 2 to about 8 thymines, about 2 to about 7 thymines, about 2 to about 6 thymines, about 2 to about 5 thymines, about 2 to about 4 thymines, about 2 to about 3 thymines.

In some embodiments, a polynucleotide disclosed herein comprises a promoter sequence (e.g., T7, SP3, or SP6 as disclosed herein) and a promoter proximal sequence comprising at least 2 thymines, at least 3 thymines, at least 4 thymines, at least 5 thymines, at least 6 thymines, at least 7 thymines, at least 8 thymines, at least 9 thymines, at least 10 thymines, at least 11 thymines, at least 12 thymines, at least 13 thymines, at least 14 thymines, at least 15 thymines, at least 16 thymines, at least 17 thymines, at least 18 thymines, at least 19 thymines, at least 20 thymines.

In some embodiments, a polynucleotide disclosed herein comprises a sequence provided in any one of Tables 1-3. In some embodiments, a polynucleotide herein comprises a sequence of SEQ ID NO: 11. In some embodiments, a polynucleotide herein comprises a sequence of SEQ ID NO: 12. In some embodiments, a polynucleotide herein comprises a sequence of SEQ ID NO: 13. In some embodiments, a polynucleotide herein comprises a sequence of SEQ ID NO: 75. In some embodiments, a polynucleotide herein comprises a sequence of SEQ ID NO: 76. In some embodiments, a polynucleotide herein comprises a sequence of SEQ ID NO: 77. In some embodiments, a polynucleotide herein comprises a sequence of SEQ ID NO: 21. In some embodiments, a polynucleotide herein comprises a sequence of SEQ ID NO: 22. In some embodiments, a polynucleotide herein comprises a sequence of SEQ ID NO: 23. In some embodiments, a polynucleotide herein comprises a sequence of SEQ ID NO: 85. In some embodiments, a polynucleotide herein comprises a sequence of SEQ ID NO: 86. In some embodiments, a polynucleotide herein comprises a sequence of SEQ ID NO: 87. In some embodiments, a polynucleotide herein comprises a sequence of SEQ ID NO: 31. In some embodiments, a polynucleotide herein comprises a sequence of SEQ ID NO: 32. In some embodiments, a polynucleotide herein comprises a sequence of SEQ ID NO: 33. In some embodiments, a polynucleotide herein comprises a sequence of SEQ ID NO: 64. In some embodiments, a polynucleotide herein comprises a sequence of SEQ ID NO: 65. In some embodiments, a polynucleotide herein comprises a sequence of SEQ ID NO: 66.

TABLE 1
Exemplary promoter proximal sequences with T7 promoter

| | | SEQ ID |
|---|---|---|
| Description | Nucleotide Sequence | NO: |
| T7 promoter (AGG) | GAATTTAATACGACTCACTATAAGG | 6 |
| T7 (AGG) + 3As | GAATTTAATACGACTCACTATAAGGAAA | 7 |
| T7 (AGG) + 6As | GAATTTAATACGACTCACTATAAGGAAAAAA | 8 |
| T7 (AGG) + 9As | GAATTTAATACGACTCACTATAAGGAAAAAAAAAAAA | 9 |
| T7 (AGG) + 12As | GAATTTAATACGACTCACTATAAGGAAAAAAAAAAAAAAA | 10 |
| T7 (AGG) + 3Ts | GAATTTAATACGACTCACTATAAGGTTT | 11 |
| T7 (AGG) + 6Ts | GAATTTAATACGACTCACTATAAGGTTTTTT | 12 |
| T7 (AGG) + 12Ts | GAATTTAATACGACTCACTATAAGGTTTTTTTTTTTT | 13 |
| T7 (AGG) + 3Cs | GAATTTAATACGACTCACTATAAGGCCC | 14 |
| T7 (AGG) + 6Cs | GAATTTAATACGACTCACTATAAGGCCCCCC | 15 |
| T7 (AGG) + 12Cs | GAATTTAATACGACTCACTATAAGGCCCCCCCCCCCC | 16 |
| T7 promoter (GGG) | GAATTTAATACGACTCACTATAGGG | 70 |
| T7 (GGG) + 3As | GAATTTAATACGACTCACTATAGGGAAA | 71 |
| T7 (GGG) + 6As | GAATTTAATACGACTCACTATAGGGAAAAAA | 72 |
| T7 (GGG) + 9As | GAATTTAATACGACTCACTATAGGGAAAAAAAAA | 73 |
| T7 (GGG) + 12As | GAATTTAATACGACTCACTATAGGGAAAAAAAAAAAA | 74 |
| T7 (GGG) + 3Ts | GAATTTAATACGACTCACTATAGGGTTT | 75 |
| T7 (GGG) + 6Ts | GAATTTAATACGACTCACTATAGGGTTTTTT | 76 |
| T7 (GGG) + 12Ts | GAATTTAATACGACTCACTATAGGGTTTTTTTTTTTT | 77 |
| T7 (GGG) + 3Cs | GAATTTAATACGACTCACTATAGGGCCC | 78 |
| T7 (GGG) + 6Cs | GAATTTAATACGACTCACTATAGGGCCCCCC | 79 |
| T7 (GGG) + 12Cs | GAATTTAATACGACTCACTATAGGGCCCCCCCCCCCC | 80 |

TABLE 2
Exemplary promoter proximal sequences with T3 promoter

| | | SEQ ID |
|---|---|---|
| Description | Nucleotide Sequence | NO: |
| T3 promoter (AGG) | AATTAACCCTCACTAAAAGG | 17 |
| T3 (AGG) + 3As | AATTAACCCTCACTAAAAGGAAA | 18 |
| T3 (AGG) + 6As | AATTAACCCTCACTAAAAGGAAAAAA | 19 |
| T3 (AGG) + 12As | AATTAACCCTCACTAAAAGGAAAAAAAAAAAAAAA | 20 |
| T3 (AGG) + 3Ts | AATTAACCCTCACTAAAAGGTTT | 21 |
| T3 (AGG) + 6Ts | AATTAACCCTCACTAAAAGGTTTTTT | 22 |
| T3 (AGG) + 12Ts | AATTAACCCTCACTAAAAGGTTTTTTTTTTTT | 23 |
| T3 (AGG) + 3Cs | AATTAACCCTCACTAAAAGGCCC | 24 |
| T3 (AGG) + 6Cs | AATTAACCCTCACTAAAAGGCCCCCC | 25 |
| T3 (AGG) + 12Cs | AATTAACCCTCACTAAAAGGCCCCCCCCCCCC | 26 |
| T3 promoter (GGG) | AATTAACCCTCACTAAAGGG | 81 |
| T3 (GGG) + 3As | AATTAACCCTCACTAAAGGGAAA | 82 |
| T3 (GGG) + 6As | AATTAACCCTCACTAAAGGGAAAAAA | 83 |
| T3 (GGG) + 12As | AATTAACCCTCACTAAAGGGAAAAAAAAAAAA | 84 |
| T3 (GGG) + 3Ts | AATTAACCCTCACTAAAGGGTTT | 85 |
| T3 (GGG) + 6Ts | AATTAACCCTCACTAAAGGGTTTTTT | 86 |
| T3 (GGG) + 12Ts | AATTAACCCTCACTAAAGGGTTTTTTTTTTTT | 87 |
| T3 (GGG) + 3Cs | AATTAACCCTCACTAAAGGGCCC | 88 |
| T3 (GGG) + 6Cs | AATTAACCCTCACTAAAGGGCCCCCC | 89 |
| T3 (GGG) + 12Cs | AATTAACCCTCACTAAAGGGCCCCCCCCCCCC | 90 |

TABLE 3
Exemplary promoter proximal sequences with SP6 promoter

| | | SEQ |
|---|---|---|
| | | ID |
| Description | Nucleotide Sequence | NO: |
| SP6 promoter (AGG) | ATTTAGGTGACACTATAAGG | 27 |
| SP6 (AGG) + 3As | ATTTAGGTGACACTATAAGGAAA | 28 |
| SP6 (AGG) + 6As | ATTTAGGTGACACTATAAGGAAAAAA | 29 |
| SP6 (AGG) + 12As | ATTTAGGTGACACTATAAGGAAAAAAAAAAAAAAA | 30 |
| SP6 (AGG) + 3Ts | ATTTAGGTGACACTATAAGGTTT | 31 |
| SP6 (AGG) + 6Ts | ATTTAGGTGACACTATAAGGTTTTTT | 32 |
| SP6 (AGG) + 12Ts | ATTTAGGTGACACTATAAGGTTTTTTTTTTTT | 33 |
| SP6 (AGG) + 3Cs | ATTTAGGTGACACTATAAGGCCC | 34 |
| SP6 (AGG) + 6Cs | ATTTAGGTGACACTATAAGGCCCCCC | 35 |
| SP6 (AGG) + 12Cs | ATTTAGGTGACACTATAAGGCCCCCCCCCCCC | 36 |
| SP6 promoter (AGA) | ATTTAGGTGACACTATAAGA | 60 |
| SP6 (AGA) + 3As | ATTTAGGTGACACTATAAGAAAA | 61 |
| SP6 (AGA) + 6As | ATTTAGGTGACACTATAAGAAAAAAA | 62 |
| SP6 (AGA) + 9As | ATTTAGGTGACACTATAAGAAAAAAAAAAAAA | 63 |
| SP6 (AGA) + 3Ts | ATTTAGGTGACACTATAAGATTT | 64 |
| SP6 (AGA) + 6Ts | ATTTAGGTGACACTATAAGATTTTTT | 65 |
| SP6 (AGA) + 12Ts | ATTTAGGTGACACTATAAGATTTTTTTTTTTT | 66 |
| SP6 (AGA) + 3Cs | ATTTAGGTGACACTATAAGACCC | 67 |
| SP6 (AGA) + 6Cs | ATTTAGGTGACACTATAAGACCCCCC | 68 |
| SP6 (AGA) + 12Cs | ATTTAGGTGACACTATAAGACCCCCCCCCCCC | 69 |

Characterization of Polyribonucleotides Made with Polynucleotides Comprising Promoter Proximal Sequences

This disclosure also provide polyribonucleotides synthesized, e.g., using fin vitro transcription (IVT) reactions from polynucleotides disclosed herein.

Polyribonucleotides made from polynucleotides disclosed herein have reduced immunogenicity when administered to a cell, tissue or subject compared to immunogenicity from polyribonucleotides made from comparable polynucleotides without a promoter proximal sequence disclosed herein.

In some embodiments, reduced immunogenicity is about 1.5-fold, 2-fold, 4-fold, 6-fold, 8-fold, 10-fold or 20-fold lesser compared to immunogenicity from polyribonucleotides made from comparable polynucleotides without a promoter proximal sequence disclosed herein.

In some embodiments, reduced immunogenicity is observed with polyribonucleotides having unmodified nucleotides.

In some embodiments, reduced immunogenicity is observed with polyribonucleotides having modified nucleotides (e.g., Ac4C, 5hmU, N1-methylpseudouridine, or combinations thereof).

In some embodiments, reduced immunogenicity is observed with polyribonucleotides having 2′O ribose modifications.

In some embodiments, reduced immunogenicity is observed with polyribonucleotides comprising less than 12 poly-adenines (e.g., synthesized from polynucleotides having a promoter and a promoter proximal sequence having less than 12 poly-adenines).

In some embodiments, reduced immunogenicity is observed with polyribonucleotides comprising less than 12 poly-cytosines (e.g., synthesized from polynucleotides having a promoter and a promoter proximal sequence having less than 12 poly-cytosines).

In some embodiments, reduced immunogenicity is observed with polyribonucleotides comprising less than 12 poly-thymines (e.g., synthesized from polynucleotides having a promoter and a promoter proximal sequence having less than 12 poly-thymines).

In some embodiments, reduced immunogenicity is observed with polyribonucleotides having Ac4C, 5hmU or both. In some embodiments, polyribonucleotides having Ac4C, 5hmU or both have a poly-adenine tract comprising two or more adenines.

In some embodiments, polyribonucleotides having Ac4C, 5hmU or both have a poly-cytosine tract comprising two or more cytosine. In some embodiments, polyribonucleotides having Ac4C, 5hmU or both have a poly-thymine tract comprising two or more thymines.

In some embodiments, polyribonucleotides made from polynucleotides disclosed herein have similar cell viability when administered to a cell compared to cell viability from polyribonucleotides made from comparable polynucleotides without a promoter proximal sequence disclosed herein.

In some embodiments, polyribonucleotides made from polynucleotides disclosed herein have similar payload expression when administered to a cell, tissue or subject compared to payload expression from polyribonucleotides made from comparable polynucleotides without a promoter proximal sequence disclosed herein.

In some embodiments, polyribonucleotides made from polynucleotides disclosed herein have increased yield as compared to polyribonucleotides made from comparable polynucleotides without a promoter proximal sequence disclosed herein. In some embodiments, yield of a polyribonucleotide made from a polynucleotide disclosed herein is at least 1.5-fold, at least 2-fold, at least 3-fold, at least 4-fold, at least 5-fold, at least 6-fold, at least 7-fold, at least 8-fold, at least 9-fold, at least 10-fold, at least 15-fold, at least 20-fold, or at least 50-fold more than the yield of a polyribonucleotide made from a comparable polynucleotide without a promoter proximal sequence disclosed herein.

In some embodiments, yield of a polyribonucleotide made from a polynucleotide disclosed herein is about 1.5-fold, about 2-fold, about 3-fold, about 4-fold, about 5-fold, about 6-fold, about 7-fold, about 8-fold, about 9-fold, about 10-fold, about 15-fold, about 20-fold, about 50-fold more than the yield of a polyribonucleotide made from a comparable polynucleotide without a promoter proximal sequence disclosed herein.

In some embodiments, yield of a polyribonucleotide made from a polynucleotide disclosed herein is about 1.5-fold to about 50-fold, about 1.5-fold to about 40-fold, about 1.5-fold to about 30-fold, about 1.5-fold to about 20-fold, about 1.5-fold to about 10-fold, about 1.5-fold to about 5 fold, about 1.5-fold to about 4-fold, about 1.5-fold to about 3-fold, or about 1.5-fold to about 2-fold.

In some embodiments, polyribonucleotides made from polynucleotides disclosed herein have similar purity as compared to polyribonucleotides made from comparable polynucleotides without a promoter proximal sequence disclosed herein.

Modified Ribonucleotides: Ac4C and 5-hmU

Polynucleotides disclosed herein can be a polyribonucleotide which further comprises one or more modified ribonucleotides comprising: a modified nucleobase, a modified ribose, a modified backbone, or any combination thereof.

In some embodiments, a polynucleotide is a polyribonucleotide. In some embodiments, a polyribonucleotide comprises one or more modified ribonucleotides. In some embodiments, a polyribonucleotide comprises a modified nucleobase, a modified ribose, a modified backbone, or any combination thereof.

In some embodiments, a polyribonucleotide comprises one or more modified nucleosides or nucleobases.

In some embodiments, a modified ribonucleotides comprises an N4-acetylcytidine nucleoside. In some embodiments, the nucleoside has the following structure:

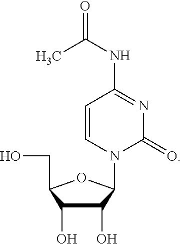

In some embodiments, a modified ribonucleotide comprises a 5-hydroxymethyluridine nucleoside. In some embodiments, the nucleoside has the following structure:

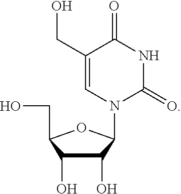

In some embodiments, a modified ribonucleotide has a structure provided in Table 4. In some embodiments, a modified ribonucleotide has a structure provided in Table 5 when incorporated in a polynucleotide. In some embodiments, a polynucleotide (e.g., a polyribonucleotide) comprises one or more modified ribonucleotides that have a structure provided in Table 5.

TABLE 4
Exemplary modified ribonucleotides
N4-acetylcytidine: 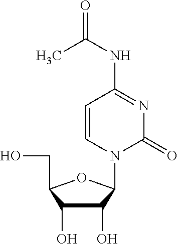
N4-acetylcytidine monophosphate: 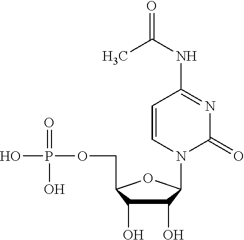
N4-acetylcytidine diphosphate: 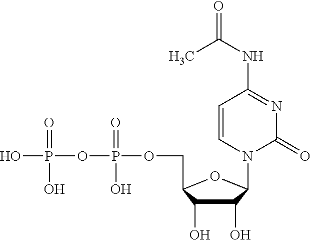
N4-acetylcytidine triphosphate: 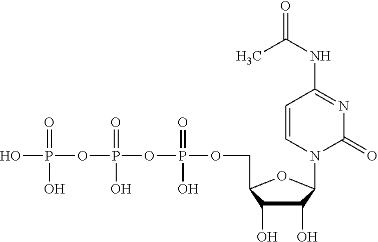
5-hydroxymethyluridine: 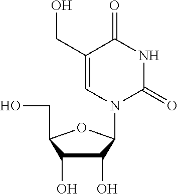
5-hydroxymethyluridine monophosphate: 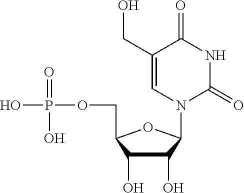
5-hydroxymethyluridine diphosphate: 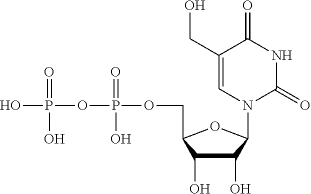
5-hydroxymethyluridine triphosphate:

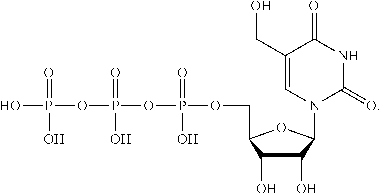

TABLE 5
Exemplary modified ribonucleotides
(when incorporated into a polyribonucleotide)
N4-acetylcytidine: 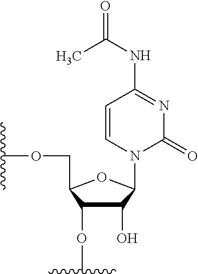
N4-acetylcytidine monophosphate: 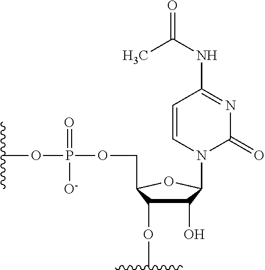
N4-acetylcytidine diphosphate: 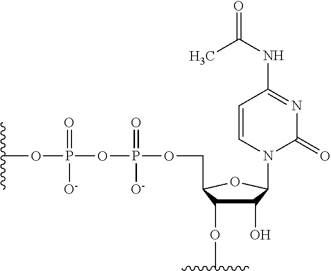
N4-acetylcytidine triphosphate: 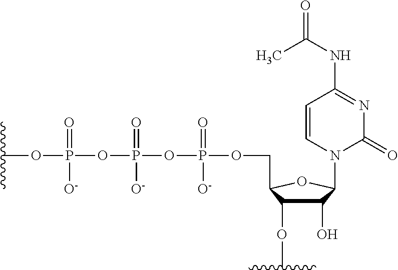
5-hydroxymethyluridine: 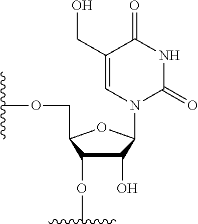
5-hydroxymethyluridine monophosphate: 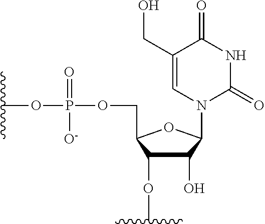
5-hydroxymethyluridine diphosphate: 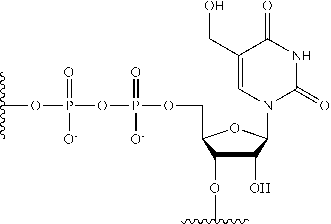
5-hydroxymethyluridine triphosphate: 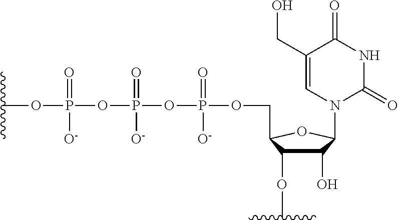
Note: [symbol] indicates the position of attachment to an adjacent ribonucleotide.

In some embodiments, a polyribonucleotide comprises cytidine nucleosides. In some embodiments, at least 5% of cytidine nucleosides in a polyribonucleotide comprise N4-acetylcytidine. In some embodiments, less than 100% of cytidine nucleosides in a polyribonucleotide comprise N4-acetylcytidine. In some embodiments, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% of cytidine nucleosides in a polyribonucleotide comprise N4-acetylcytidine.

In some embodiments, a polyribonucleotide comprises uridine nucleosides. In some embodiments, at least 5% of uridine residues in a polyribonucleotide comprise 5-hydroxymethyluridine. In some embodiments, less than 100% of uridine nucleosides in a polyribonucleotide comprise 5-hydroxymethyluridine. In some embodiments, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99% of uridine nucleosides in a polyribonucleotide comprise 5-hydroxymethyluridine. In some embodiments, more than 60% of uridine nucleosides in a polyribonucleotide comprise 5-hydroxymethyluridine.

In some embodiments, a polyribonucleotide disclosed herein comprises one or more modified ribonucleotides comprises a nucleoside comprising: N4-acetylcytidine, 5-hydroxymethyluridine, N1-methylpseudouridine, pyridin-4-one ribonucleoside, 5-aza-uridine, 6-aza-uridine, 2-thio-5-aza-uridine, 2-thio-uridine (s2U), 5-methyl cytidine (m5C), 5-aza-cytidine, 6-aza-cytidine, pseudoisocytidine, 3-methyl-cytidine (m3C), 5-formyl-cytidine (f5C), N4-methyl-cytidine (m4C), 2-amino-purine, 2, 6-diaminopurine, 2-amino-6-halo-purine, 6-halo-purine, inosine (I), 1-methyl-inosine (m1 I), wyosine (imG), methylwyosine (mimG), 5-hydroxycytidine, 5-hydroxymethylcytidine, 5-carboxycytidine, 5-methoxycytidine, 5-propynylcytidine, 2-thiocytidine, 5-hydroxyuridine, 5-methyluridine, 5,6-dihydro-5-methyluridine, 2′-O-methyluridine, 2′-O-methyl-5-methyluridine, 2′-fluoro-2′-deoxyuridine, 2′-amino-2′-deoxyuridine, 2′-azido-2′-deoxyuridine, 4-thiouridine, 5-carboxyuridine, 5-carboxymethylesteruridine, 5-formyluridine, 5-methoxyuridine, 5-propynyluridine, 5-bromouridine, 5-iodouridine, 5-fluorouridine, pseudouridine, 2′-O-methyl-pseudouridine, N1-hydroxypseudouridine, 2′-O-methyl-N1-methylpseudouridine, N1-ethylpseudouridine, N1-hydroxymethylpseudouridine, ara-uridine, N6-methyladenosine, 2-aminoadenosine, 3-methyladenosine, 7-deazaadenosine, 8-oxoadenosine, thienoguanosine, 7-deazaguanosine, 8-oxoguanosine, 6-O-methylguanine, or any combination thereof.

### Modified Ribose

Polynucleotides disclosed herein can be a polyribonucleotide which further comprises one or more modified ribonucleotides comprising: a modified nucleobase, a modified ribose, a modified backbone, or any combination thereof.

In some embodiments, a polynucleotide is a polyribonucleotide. In some embodiments, a polyribonucleotide comprises one or more modified ribonucleotides. In some embodiments, a modified ribonucleotide comprises a modified ribose.

In some embodiments, a modified ribonucleotide comprises a 2′-O-acetylated ribose. In some embodiments, a modified ribonucleotide comprises a structure of:

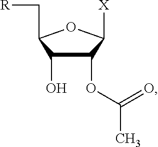

(a) wherein R is a monophosphate, a diphosphate, or a triphosphate; and
(b) wherein X is an adenine nucleobase, a guanine nucleobase, a cytosine nucleobase (e.g., N4-acetylcytosine), or a uracil nucleobase (e.g., 5-hydroxymethyluracil).

In some embodiments, a modified ribonucleotide comprises a 2′-O-acetylated ribose and an adenine nucleobase.

In some embodiments, a modified ribonucleotide comprises a 2′-O-acetylated ribose and a guanine nucleobase.

In some embodiments, a modified ribonucleotide comprises a 2′-O-acetylated ribose and a cytosine nucleobase.

In some embodiments, a modified ribonucleotide comprises a 2′-O-acetylated ribose and a N4-acetylcytidine nucleoside.

In some embodiments, a modified ribonucleotide comprises a 2′-O-acetylated ribose and a uracil nucleobase.

In some embodiments, a modified ribonucleotide comprises a 2′-O-acetylated ribose and a 5-hydroxymethyluridine nucleoside.

In some embodiments, a modified ribonucleotide comprises a 2′-O-acetylated ribose and a N1-methylpseudouracil nucleobase.

In some embodiments, a modified ribonucleotide comprises a structure provided in Table 6. In some embodiments, a modified ribonucleotide comprises a structure provided in Table 7 when incorporated in a polynucleotide. In some embodiments, a polynucleotide (e.g., a polyribonucleotide) comprises one or more modified ribonucleotides that have a structure provided in Table 7.

TABLE 6
Exemplary modified ribonucleotides
2′-O-acetyl N4-acetylcytidine: 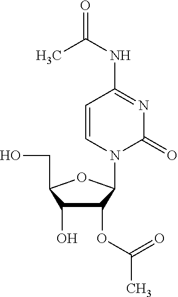
2′-O-acetyl N4-acetylcytidine monophosphate: 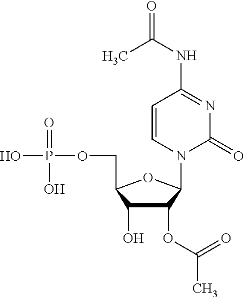
2′-O-acetyl N4-acetylcytidine diphosphate: 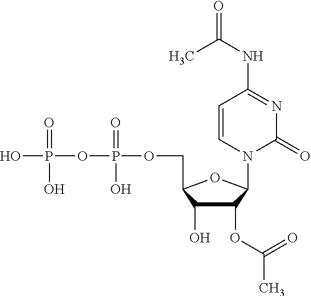
2′-O-acetyl N4-acetylcytidine triphosphate: 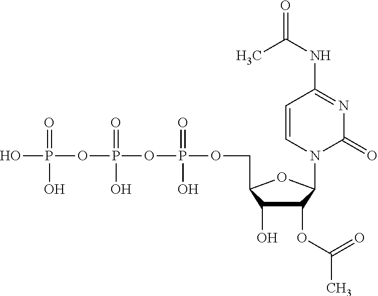
2′-O-acetyl 5- hydroxymethyluridine: 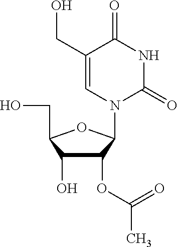
2′-O-acetyl 5-hydroxymethyluridine monophosphate: 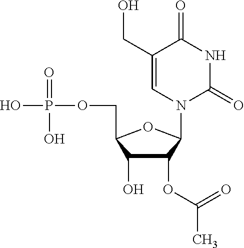
2′-O-acetyl 5- hydroxymethyluridine diphosphate: 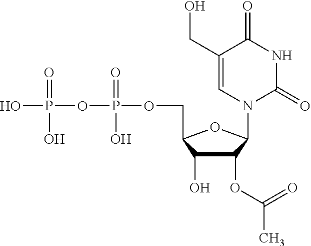
2′-O-acetyl 5-hydroxymethyluridine triphosphate: 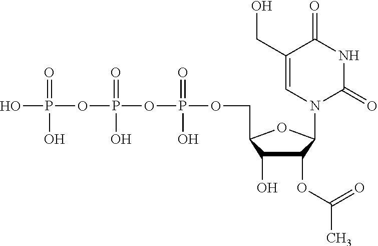
2′-O-acetyl-1-methylpseudouridine 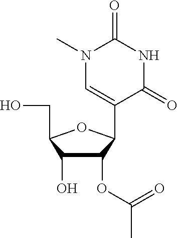
2′-O-acetyl-1-methylpseudouridine monophosphate 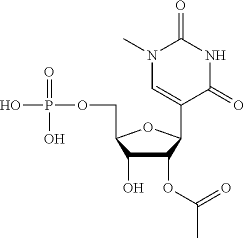
2′-O-acetyl-1-methylpseudouridine diphosphate 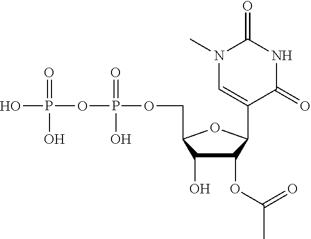
2′-O-acetyl-1-methylpseudouridine triphosphate 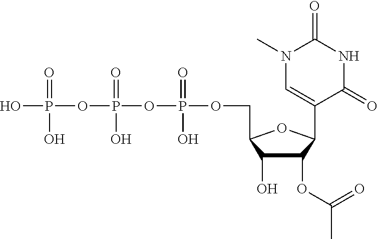
2′-O-acetyluridine: 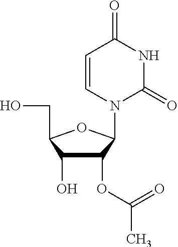
2′-O-acetyluridine monophosphate: 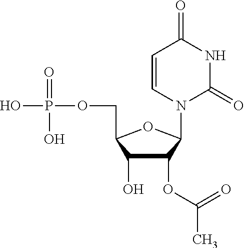
2′-O-acetyluridine diphosphate: 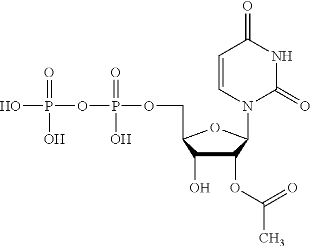
2′-O-acetyluridine triphosphate: 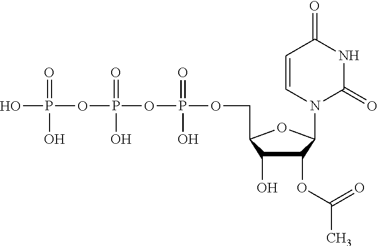
2′-O-acetylcytidine: 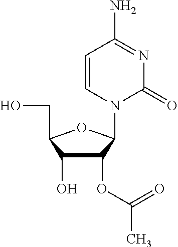
2′-O-acetylcytidine monophosphate: 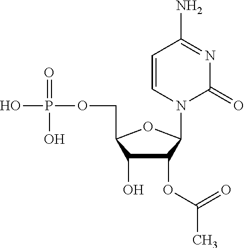
2′-O-acetylcytidine diphosphate: 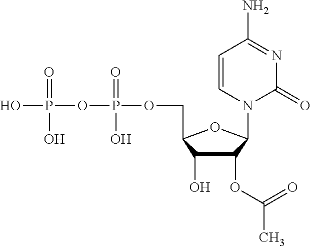
2′-O-acetylcytidine triphosphate: 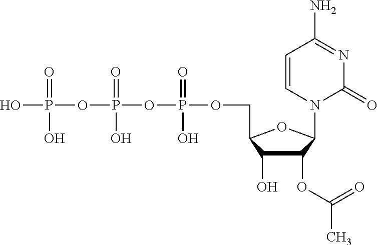
2′-O-acetylguanosine: 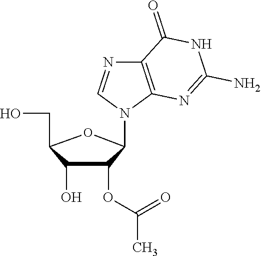
2′-O-acetylguanosine monophosphate: 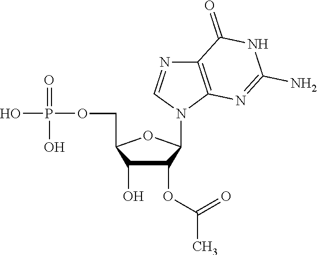
2′-O-acetylguanosine diphosphate: 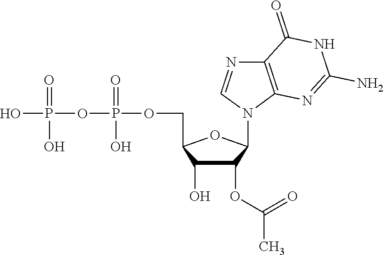
2′-O-acetylguanosine triphosphate: 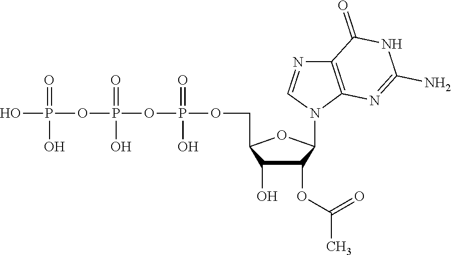
2′-O-acetyladenosine: 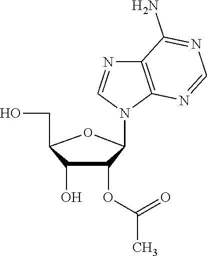
2′-O-acetyladenosine monophosphate: 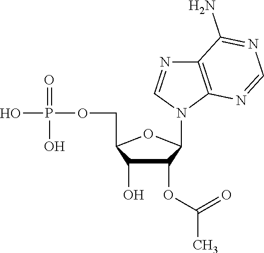
2′-O-acetyladenosine diphosphate: 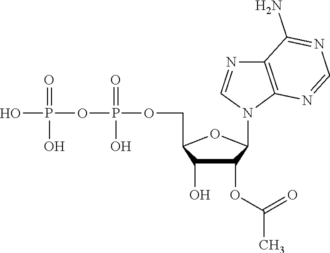
2′-O-acetyladenoosine triphosphate:

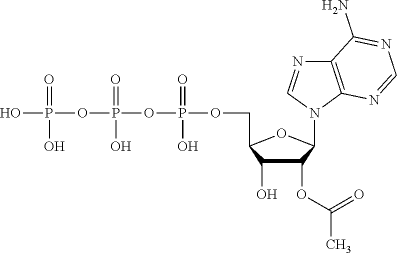

TABLE 7
Exemplary modified ribonucleotides
(when incorporated into a polyribonucleotide)
2′-O-acetyl N4-acetylcytidine: 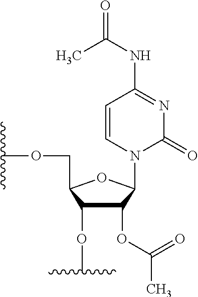
2′-O-acetyl N4-acetylcytidine monophosphate: 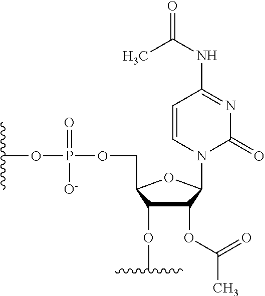
2′-O-acetyl N4-acetylcytidine diphosphate: 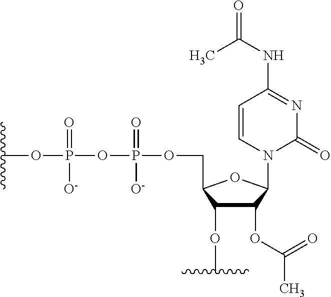
2′-O-acetyl N4-acetylcytidine triphosphate: 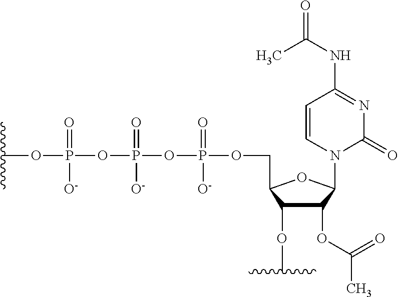
2′-O-acetyl 5- hydroxymethyluridine: 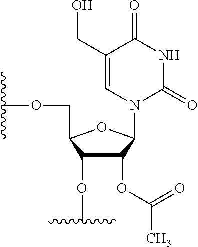
2′-O-acetyl 5- hydroxymethyluridine monophosphate: 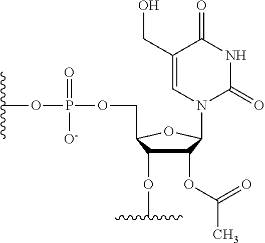
2′-O-acetyl 5- hydroxymethyluridine diphosphate: 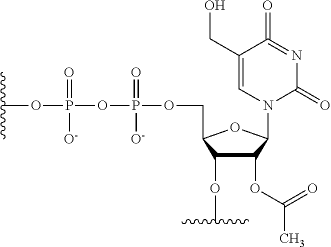
2′-O-acetyl 5- hydroxymethyluridine triphosphate: 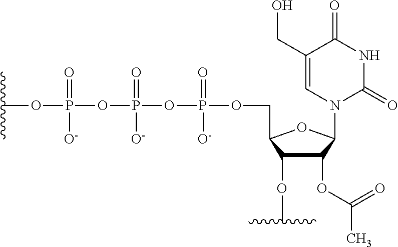
2′-O-acetyluridine: 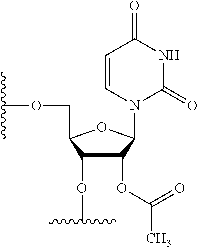
2′-O-acetyluridine monophosphate: 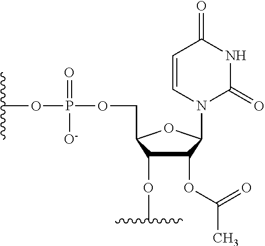
2′-O-acetyluridine diphosphate: 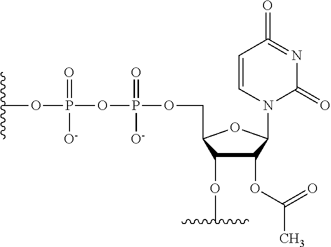
2′-O-acetyluridine triphosphate: 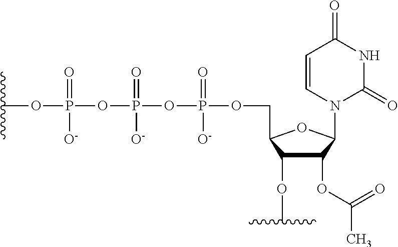
2′-O-acetylcytidine: 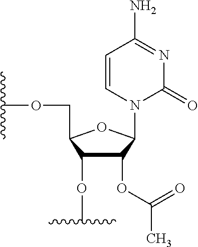
2′-O-acetylcytidine monophosphate: 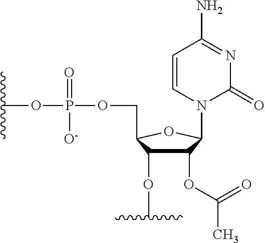
2′-O-acetylcytidine diphosphate: 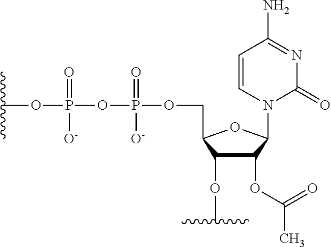
2′-O-acetylcytidine triphosphate: 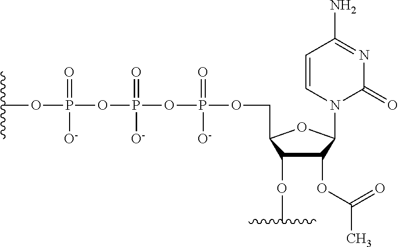
2′-O-acetylguanosine: 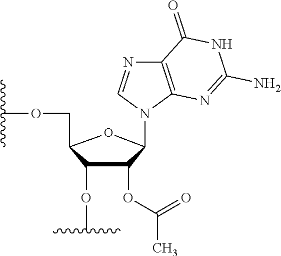
2′-O-acetylguanosine monophosphate: 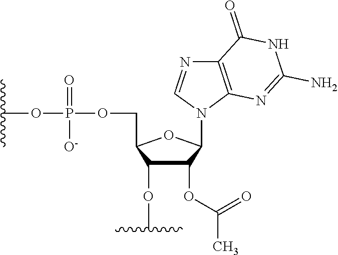
2′-O-acetylguanosine diphosphate: 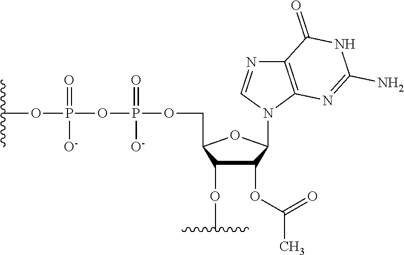
2′-O-acetylguanosine triphosphate: 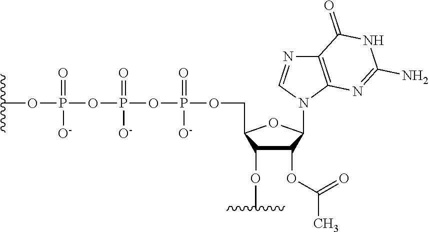
2′-O-acetyladenosine: 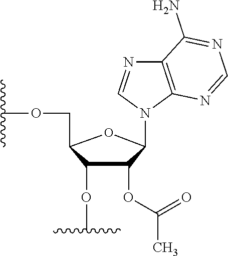
2′-O-acetyladenosine monophosphate: 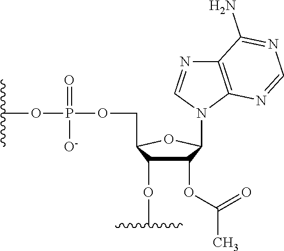
2′-O-acetyladenosine diphosphate: 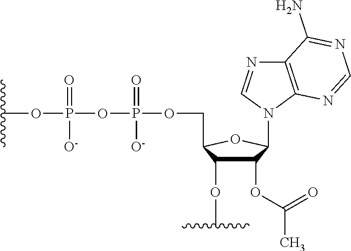
2′-O-acetyladenoosine triphosphate: 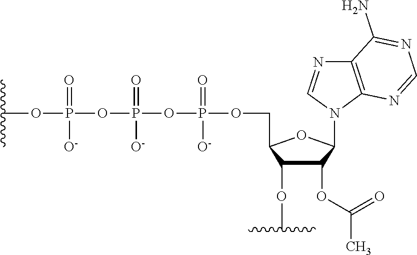
2′-O-acetyl-1- methylpseudouridine 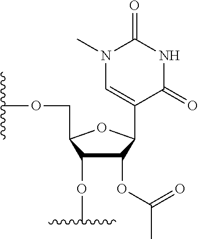
2′-O-acetyl-1- methylpseudouridine monophosphate 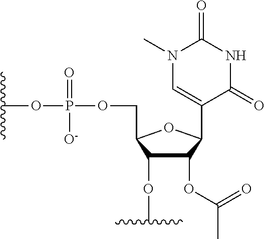
2′-O-acetyl-1- methylpseudouridine diphosphate 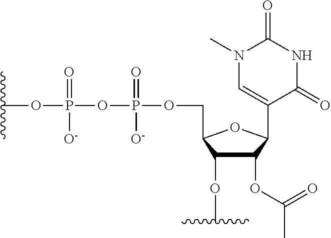
2′-O-acetyl-1- methylpseudouridine triphosphate 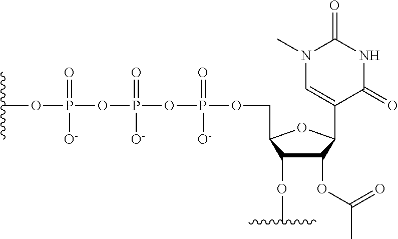
NOTE: [symbol] indicates the position of attachment to an adjacent ribonucleotide.

In some embodiments, at least 5% of ribose moieties of a polyribonucleotide are 2′-O-acetylated.

In some embodiments, about 5% to about 99% of the ribose moieties of a polyribonucleotide are 2′-O-acetylated.

In some embodiments, a polyribonucleotide disclosed herein comprises a cap structure and the cap structure does not comprise a 2′-O-acetylated ribose.

In some embodiments, a polyribonucleotide disclosed herein comprises a cap structure and the cap structure comprises a 2′-O-acetylated ribose.

In some embodiments, a polyribonucleotide disclosed herein further comprises one or more ribonucleotides that does not comprise a 2′-0 acetylated ribose.

### Cap Structures

In some embodiments of any of the polyribonucleotides disclosed herein, a polyribonucleotide comprises a cap structure.

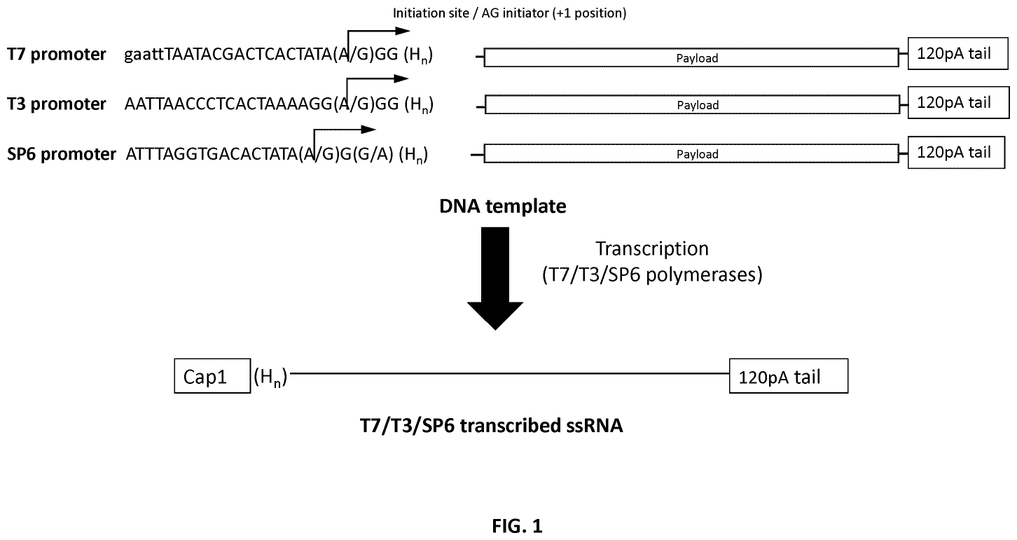

Prior versions of RNA capping have been described, e.g., in Decroly E et al. (2012) Nature Reviews 10: 51-65; and in Ramanathan A. et al., (2016) Nucleic Acids Res, 44(16): 7511-7526, the entire contents of each of which is hereby incorporated by reference. 5′ caps include a Cap-0 (also referred herein as “Cap0”, a Cap-1 (also referred herein as “Cap1”, or Cap-2 (also referred herein as “Cap2”). See, e.g., FIG. 1 of Ramanathan A et al., and FIG. 1 of Decroly E et al.

The term “5′-cap” as used herein refers to a structure found on the 5′-end of an RNA, e.g., Mrna, and generally includes a guanosine nucleotide connected to an RNA, e.g., Mrna, via a 5′- to 5′-triphosphate linkage (also referred to as Gppp or G(5′ppp(5)).

In some embodiments of a polyribonucleotide disclosed herein, the 5′ end of the polyribonucleotide comprises a compound of formula I:

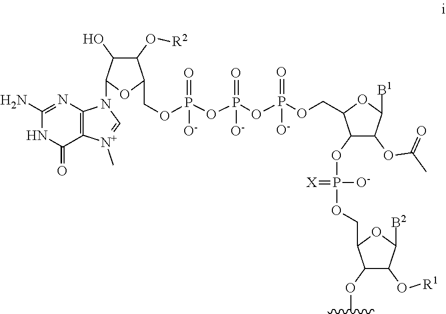

or a salt thereof, wherein:
[symbol] indicates the position at which the compound is attached to the polyribonucleotide;
each of B¹ and B² is independently selected from a natural base and a modified base;
R¹ is selected from hydrogen, C₁₋₆ alkyl, and —C(═O)CH₃;
R² is hydrogen or —CH₃; and
X is O or S.

As defined generally above for formula I, each of B¹ and B² is independently selected from a natural base and a modified base. In some embodiments, B¹ is a natural base. In some embodiments, B¹ is adenine. In some embodiments, B¹ is cytosine. In some embodiments, B¹ is guanine. In some embodiments, B¹ is uracil.

In some embodiments, B¹ is a modified base. In some embodiments, B¹ is selected from N1-methylpseudouracil, a pyridin-4-one ribonucleoside, 5-aza-uracil, 6-aza-uracil, 2-thio-5-aza-uracil, 2-thio-uracil, 5-methyl cytosine, 5-aza-cytosine, 6-aza-cytosine, pseudoisocytosine, 3-methyl-cytosine, 5-formyl-cytosine, N4-methyl-cytosine, a 2-amino-purine, a 2,6-diaminopurine, a 2-amino-6-halo-purine, a 6-halo-purine, hypoxanthine, 1-methyl-hypoxanthine, 4,6-dimethyl-3,3a,4,9a-tetrahydro-9H-imidazo[1,2-a]purin-9-one, 4,6,7-trimethyl-3,3a,4,9a-tetrahydro-9H-imidazo[1,2-a]purin-9-one, N4-acetylcytosine, and 5-hydroxymethyluracil.

In some embodiments, B² is a natural base. In some embodiments, B² is adenine. In some embodiments, B² is cytosine. In some embodiments, B² is guanine. In some embodiments, B² is uracil.

In some embodiments, B² is a modified base. In some embodiments, B² is selected from N1-methylpseudouracil, a pyridin-4-one ribonucleoside, 5-aza-uracil, 6-aza-uracil, 2-thio-5-aza-uracil, 2-thio-uracil, 5-methyl cytosine, 5-aza-cytosine, 6-aza-cytosine, pseudoisocytosine, 3-methyl-cytosine, 5-formyl-cytosine, N4-methyl-cytosine, a 2-amino-purine, a 2,6-diaminopurine, a 2-amino-6-halo-purine, a 6-halo-purine, hypoxanthine, 1-methyl-hypoxanthine, 4,6-dimethyl-3,3a,4,9a-tetrahydro-9H-imidazo[1,2-a]purin-9-one, 4,6,7-trimethyl-3,3a,4,9a-tetrahydro-9H-imidazo[1,2-a]purin-9-one, N4-acetylcytosine, and 5-hydroxymethyluracil.

As defined generally above for formula I, R¹ is selected from hydrogen, C₁₋₆ alkyl, and —C(═O)CH₃. In some embodiments, R¹ is hydrogen. In some embodiments, R¹ is C₁₋₆ alkyl. In some embodiments, R¹ is —CH₃. In some embodiments, R¹ is —C(═O)CH₃.

As defined generally above for formula I, R² is hydrogen or —CH₃. In some embodiments, R² is hydrogen. In some embodiments, R² is —CH₃.

As defined generally above for formula I, X is O or S. In some embodiments, X is O. In some embodiments, X is S.

In some embodiments, the present disclosure provides a compound of any of formulae I-a, I-b, I-c, I-d, I-e, and I-f:

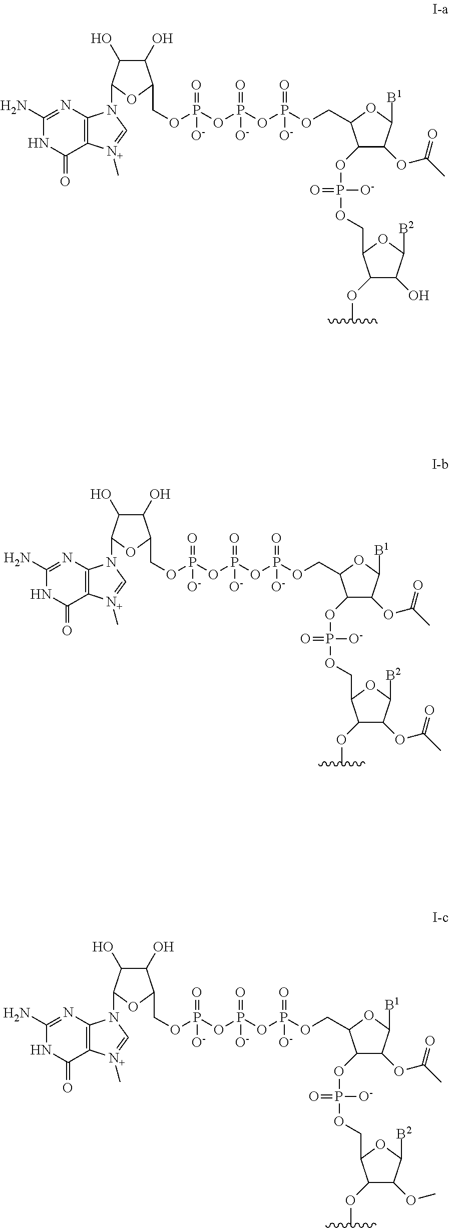

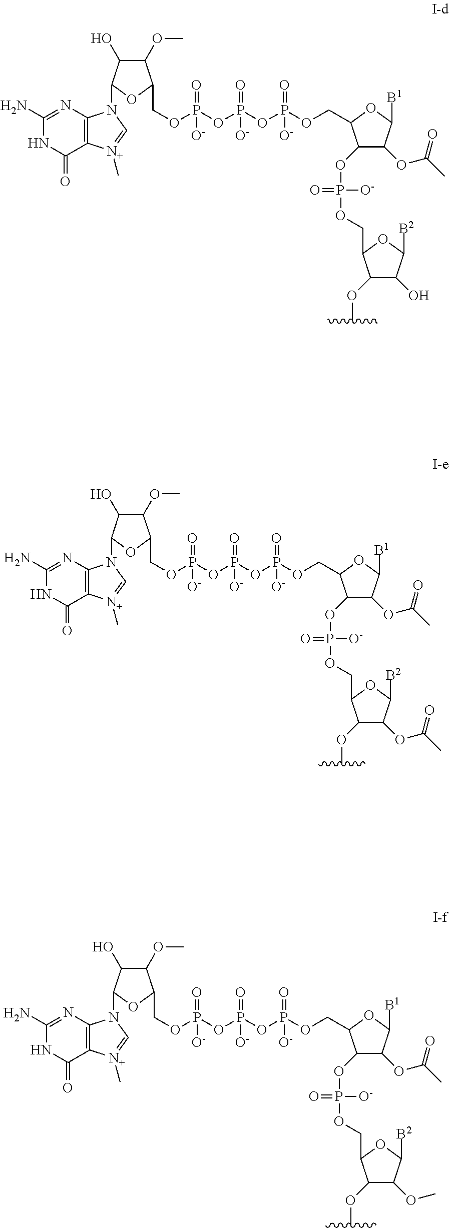

or a salt thereof, wherein each of B¹ and B² is as defined above and described herein.

In some embodiments, a compound of formula I is selected from

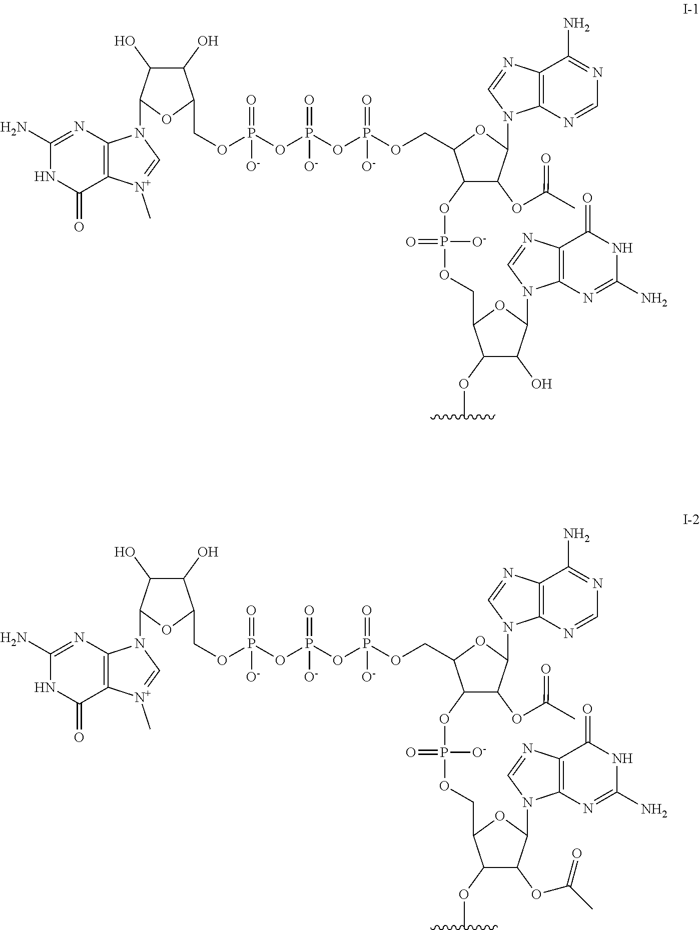 [SMILES: *OC1C(COP(=O)([O-])OC2C(COP(=O)([O-])OP(=O)([O-])OP(=O)([O-])OCC3OC(n4c[n+](C)c5c(=O)[nH]c(N)nc54)C(O)C3O)OC(n3cnc4c(N)ncnc43)C2OC(C)=O)OC(n2cnc3c(=O)[nH]c(N)nc32)C1O ; *OC1C(COP(=O)([O-])OC2C(COP(=O)([O-])OP(=O)([O-])OP(=O)([O-])OCC3OC(n4c[n+](C)c5c(=O)[nH]c(N)nc54)C(O)C3O)OC(n3cnc4c(N)ncnc43)C2OC(C)=O)OC(n2cnc3c(=O)[nH]c(N)nc32)C1OC(C)=O]

or a salt thereof.

In some embodiments, a polyribonucleotide disclosed herein comprises at the 5′ end a ^{m7}GpppN₁Pn₂-OH cap with the following structure:

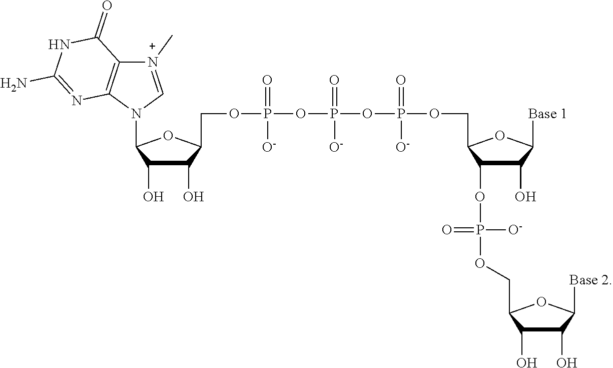

In some embodiments, a polyribonucleotide disclosed herein comprises at the 5′ end a compound with the following structure:

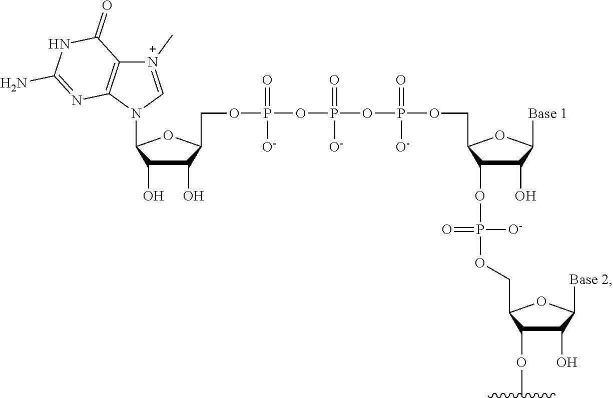

wherein [symbol] indicates the position at which the compound is attached to the polyribonucleotide.

In some embodiments, a polyribonucleotide disclosed herein comprises a ^{m7}GpppN₁^{(2′-Ome)}Pn₂-OH cap with the following structure:

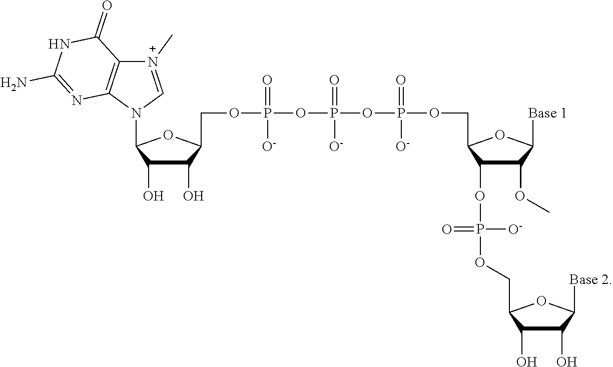

In some embodiments, a polyribonucleotide disclosed herein comprises at the 5′ end a compound with the following structure:

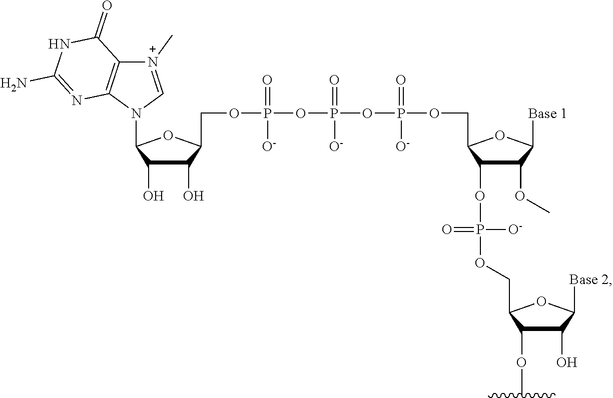

wherein [symbol] indicates the position at which the compound is attached to the polyribonucleotide.

In some embodiments, a polyribonucleotide disclosed herein comprises a ^{m7}GpppN₁^{(2′-Ome)}Pn₂^{(2′-Ome)}—OH cap with the following structure:

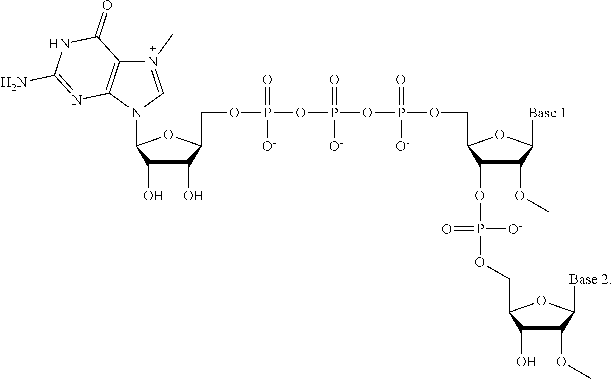

In some embodiments, a polyribonucleotide disclosed herein comprises at the 5′ end a compound with the following structure:

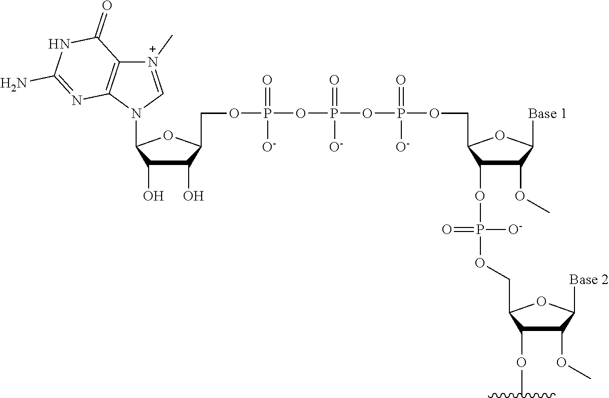

wherein [symbol] indicates the position at which the compound is attached to the polyribonucleotide.

In some embodiments, for any of the cap structures disclosed herein, each of B¹ and B² is independently selected from a natural base and a modified base. In some embodiments, B¹ is a natural base. In some embodiments, B¹ is adenine. In some embodiments, B¹ is cytosine. In some embodiments, B¹ is guanine. In some embodiments, B¹ is uracil.

In some embodiments, B¹ is a modified base. In some embodiments, B¹ is selected from N1-methylpseudouracil, a pyridin-4-one ribonucleoside, 5-aza-uracil, 6-aza-uracil, 2-thio-5-aza-uracil, 2-thio-uracil, 5-methyl cytosine, 5-aza-cytosine, 6-aza-cytosine, pseudoisocytosine, 3-methyl-cytosine, 5-formyl-cytosine, N4-methyl-cytosine, a 2-amino-purine, a 2,6-diaminopurine, a 2-amino-6-halo-purine, a 6-halo-purine, hypoxanthine, 1-methyl-hypoxanthine, 4,6-dimethyl-3,3a,4,9a-tetrahydro-9H-imidazo[1,2-a]purin-9-one, 4,6,7-trimethyl-3,3a,4,9a-tetrahydro-9H-imidazo[1,2-a]purin-9-one, N4-acetylcytosine, and 5-hydroxymethyluracil.

In some embodiments, B² is a natural base. In some embodiments, B² is adenine. In some embodiments, B² is cytosine. In some embodiments, B² is guanine. In some embodiments, B² is uracil.

In some embodiments, B² is a modified base. In some embodiments, B² is selected from N1-methylpseudouracil, a pyridin-4-one ribonucleoside, 5-aza-uracil, 6-aza-uracil, 2-thio-5-aza-uracil, 2-thio-uracil, 5-methyl cytosine, 5-aza-cytosine, 6-aza-cytosine, pseudoisocytosine, 3-methyl-cytosine, 5-formyl-cytosine, N4-methyl-cytosine, a 2-amino-purine, a 2,6-diaminopurine, a 2-amino-6-halo-purine, a 6-halo-purine, hypoxanthine, 1-methyl-hypoxanthine, 4,6-dimethyl-3,3a,4,9a-tetrahydro-9H-imidazo[1,2-a]purin-9-one, 4,6,7-trimethyl-3,3a,4,9a-tetrahydro-9H-imidazo[1,2-a]purin-9-one, N4-acetylcytosine, and 5-hydroxymethyluracil.

### Method of Making Polyribonucleotides

This disclosure provides, among other things, methods of making a polyribonucleotide using a polynucleotide disclosed herein. In some embodiments, disclosed herein is method of producing a polyribonucleotide comprising a step of incubating an in vitro transcription mixture, wherein the in vitro transcription mixture comprises: (i) a DNA template comprising a polynucleotide comprising a promoter proximal sequence disclosed herein; (ii) at least one RNA polymerase or a variant or a fragment thereof; and (iii) a plurality of ribonucleotides.

Also disclosed herein are in vitro transcription mixtures useful in producing a polyribonucleotide disclosed herein. In some embodiments, disclosed herein is in vitro transcription mixture comprising: (i) a DNA template comprising a polynucleotide comprising a promoter proximal sequence disclosed herein; (ii) at least one RNA polymerase or a variant or a fragment thereof; and (iii) a plurality of ribonucleotides.

In some embodiments, a method or an in vitro transcription mixture produces a plurality of polyribonucleotides.

In some embodiments, an RNA polymerase is chosen from: a bacteriophage RNA polymerase, a mitochondrial RNA polymerase, a eukaryotic RNA polymerase, a bacterial RNA polymerase, or any combination thereof. In some embodiments, an RNA polymerase comprises a T7 RNA polymerase, a T3 RNA polymerase, a SP6 RNA polymerase, a viral RNA polymerase, a N4 virion RNA polymerase, or a variant of any of the foregoing.

In some embodiments, a polyribonucleotide produced by a method disclosed herein or using an in vitro transcription mixture comprises a coding region. In some embodiments, a coding region encodes a gene product, e.g., as described herein.

In some embodiments, a polyribonucleotide produced by a method disclosed herein or using an in vitro transcription mixture does not comprise a coding region. In some embodiments, a polyribonucleotide which does not comprises a coding region can also be referred to as a non-coding RNA. Exemplary non-coding RNA include, but are not limited to, long non-coding RNAS (lncRNA), microRNAs, siRNAs, piRNAs, snoRNAs, snRNAs, exRNAs, scaRNAs rRNA, and tRNA,

In some embodiments, a polyribonucleotide produced by a method disclosed herein or using an in vitro transcription mixture comprises a guide RNA, a short hairpin RNA, an siRNA, a microRNA, a long non-coding RNA, a circular RNA, or a messenger RNA (mRNA), or any combination thereof.

In some embodiments, a polyribonucleotide produced by a method disclosed herein or using an in vitro transcription mixture comprises a modified nucleobase, a modified ribose, a modified backbone, or any combination thereof.

In some embodiments, a polyribonucleotide produced by a method disclosed herein or using an in vitro transcription mixture encodes a payload. In some embodiments, a payload comprises one or more target polypeptides. In some embodiments, a payload comprises an RNA situated in a polyribonucleotide.

In some embodiments, a polyribonucleotide produced by a method disclosed herein or using an in vitro transcription mixture comprises a cap structure as disclosed herein.

In some embodiments, a polyribonucleotide produced by a method disclosed herein or using an in vitro transcription mixture does not comprise a cap structure.

In some embodiments of a method of producing a polyribonucleotide disclosed herein, an incubating step occurs at a temperature of at least 37° C.

In some embodiments of a method of producing a polyribonucleotide disclosed herein, an incubating step occurs at a temperature of about 45° C., 46° C., 47° C., 48° C., 49° C., 50° C., 51° C., 52° C., 53° C., 54° C., 55° C., or higher.

In some embodiments of a method of producing a polyribonucleotide disclosed herein, an incubating step is performed for at least 1 hour, at least 2 hours, at least 3 hours, at least 4 hours or longer.

In some embodiments method of producing a polyribonucleotide disclosed herein, an incubating step is performed at a pH of: about 5 to about 8, about 5 to about 7.9, about 5 to about 7.8, about 5 to about 7.7, about 5 to about 7.6, about 5 to about 7.5, about 5 to about 7.4, about 5 to about 7.3, about 5 to about 7.2, about 5 to about 7.1, about 5 to about 7.0, about 5 to about 6.9, about 5 to about 6.8, about 5 to about 6.7, about 5 to about 6.6, about 5 to about 6.5, about 5 to about 6.4, about 5 to about 6.3, about 5 to about 6.2, about 5 to about 6.1, about 5 to about 6.0, about 5 to about 5.9, about 5 to about 5.8, about 5 to about 5.7, about 5 to about 5.6, about 5 to about 5.5, about 5 to about 5.4, about 5 to about 5.3, about 5 to about 5.2, about 5 to about 5.1, or about 5.1 to about 8, about 5.2 to about 8, about 5.3 to about 8, about 5.4 to about 8, about 5.5 to about 8, about 5.6 to about 8, about 5.7 to about 8, about 5.8 to about 8, about 5.9 to about 8, about 6.0 to about 8, about 6.1 to about 8, about 6.2 to about 8, about 6.3 to about 8, about 6.4 to about 8, about 6.5 to about 8, about 6.6 to about 8, about 6.7 to about 8, about 6.8 to about 8, about 6.9 to about 8, about 7.0 to about 8, about 7.1 to about 8, about 7.2 to about 8, about 7.3 to about 8, about 7.4 to about 8, about 7.5 to about 8, about 7.6 to about 8, about 7.7 to about 8, about 7.8 to about 8, or about 7.9 to about 8.

In some embodiments method of producing a polyribonucleotide disclosed herein, an incubating step is performed at a pH of about 5, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, or about 8.0.

### Compositions and Pharmaceutical Compositions

Among other things, provided herein are compositions comprising polynucleotides disclosed herein.

In some embodiments, a composition is a pharmaceutical composition.

Pharmaceutical compositions of the present disclosure may comprise a polynucleotide disclosed herein, or an expression vector comprising a polynucleotide. In some embodiments, a pharmaceutical composition may comprise a pharmaceutically acceptable excipient, a diluent, or a combination thereof. In some embodiments, a pharmaceutical composition may comprise buffers such as neutral buffered saline, phosphate buffered saline and the like; carbohydrates such as glucose, mannose, sucrose, or dextrans; mannitol; proteins; polypeptides or amino acids such as glycine; antioxidants; chelating agents such as EDTA or glutathione; and preservatives.

In some embodiments, a pharmaceutical composition is formulated for administration according to any of the routes of administration disclosed herein. In some embodiments, a pharmaceutical composition is formulated for intramuscular, intradermal, intravenous, subcutaneous, mucosal, inhaled, or intranodal delivery.

### Formulations

Polynucleotides disclosed herein can be polyribonucleotides. Among other things, provided herein are compositions comprising a polyribonucleotide disclosed herein, and formulations thereof. In some embodiments, a composition comprising a polyribonucleotide disclosed herein is formulated in a lipid nanoparticle (LNP) formulation.

In some embodiments, a polyribonucleotide disclosed herein encodes for a polypeptide. In some embodiments, a polyribonucleotide disclosed herein is or comprises a messenger RNA. In some embodiments, a composition comprising a polyribonucleotide comprising a messenger RNA is formulated in a lipid nanoparticle (LNP) formulation.

In some embodiments, a polyribonucleotide disclosed herein is or comprises a Grna. In some embodiments, a composition comprising a polyribonucleotide comprising a Grna is formulated in a lipid nanoparticle (LNP) formulation.

In some embodiments, a polyribonucleotide disclosed herein is or comprises an inhibitory RNA. In some embodiments, a composition comprising a polyribonucleotide comprising an inhibitory RNA is formulated in a lipid nanoparticle (LNP) formulation.

In some embodiments, a polyribonucleotide disclosed herein is or comprises an miRNA or siRNA. In some embodiments, a composition comprising a polyribonucleotide comprising a miRNA or siRNA is formulated in a lipid nanoparticle (LNP) formulation.

In some embodiments, a polyribonucleotide disclosed herein is or comprises an antisense oligonucleotide. In some embodiments, a composition comprising a polyribonucleotide comprising an antisense oligonucleotide is formulated in a lipid nanoparticle (LNP) formulation.

In some embodiments, the disclosure provides an LNP formulation comprising a polyribonucleotide disclosed herein for use in a pharmaceutical composition, e.g., an immunogenic composition.

### Methods of Using Polynucleotides or Compositions Disclosed Herein

The disclosure provides, among other things, methods for using a polynucleotide disclosed herein, or a composition comprising the same.

In some embodiments, provided herein is a method of administering a polynucleotide disclosed herein or a composition comprising a polynucleotide disclosed herein to a cell, tissue or subject.

In some embodiments, provided herein is a vaccination method comprising administering a polynucleotide disclosed herein or a composition comprising a polynucleotide disclosed herein to a cell, tissue or subject.

In some embodiments, disclosed herein is a gene therapy method comprising administering a polynucleotide disclosed herein or a composition comprising a polynucleotide disclosed herein to a cell, tissue or subject. In some embodiments, a gene therapy method comprises delivery of one or more components of a gene therapy, e.g., a guide RNA and/or a Cas polypeptide.

In some embodiments, provided herein is a method for stimulating an immune response comprising administering a polynucleotide disclosed herein or a composition comprising a polynucleotide disclosed herein to a cell, tissue or subject.

In some embodiments, also provided herein is a cell therapy engineering method comprising administering a polynucleotide disclosed herein or a composition comprising a polynucleotide disclosed herein to a cell, tissue or subject.

In some embodiments, provided herein is an immunotherapy method comprising administering a polynucleotide disclosed herein or a composition comprising a polynucleotide disclosed herein to a cell, tissue or subject. In some embodiments, an immunotherapy method comprises delivery of an antibody therapy and/or an immune checkpoint therapy.

In some embodiments, disclosed herein is a protein replacement therapy method comprising administering a polynucleotide disclosed herein or a composition comprising a polynucleotide disclosed herein to a cell, tissue or subject. In some embodiments, a protein replacement therapy comprises delivery of an enzyme replacement therapy.

In some embodiments, provided herein is a chemotherapeutic method comprising administering a polynucleotide disclosed herein or a composition comprising a polynucleotide disclosed herein to a cell, tissue or subject.

### Kits

Another aspect of the present disclosure further provides a pharmaceutical pack or kit. In some embodiments, a kit can comprise a polynucleotide or a composition described herein. In some embodiment, kits may be used in any applicable method, e.g., methods as described herein.

### Enumerated Embodiments

Embodiment 1. A recombinant polynucleotide sequence, comprising:
(i) a promoter sequence comprising a 5′ end and a 3′ end;
(ii) a promoter proximal sequence adjacent to the 3′ end of the promoter sequence, wherein the promoter proximal sequence comprises two or more consecutive nucleotides; and
(iii) a sequence encoding a target operably linked to (ii).

Embodiment 2. The recombinant polynucleotide of any one of the preceding embodiments, wherein the promoter proximal sequence comprises three or more consecutive nucleotides.

Embodiment 3. The recombinant polynucleotide of any one of the preceding embodiments, wherein the promoter proximal sequence comprises four or more consecutive nucleotides.

Embodiment 4. The recombinant polynucleotide of any one of the preceding embodiments, wherein the promoter proximal sequence comprises five or more consecutive nucleotides.

Embodiment 5. The recombinant polynucleotide of any one of the preceding embodiments, wherein the promoter proximal sequence comprises six or more consecutive nucleotides.

Embodiment 6. The recombinant polynucleotide of any one of the preceding embodiments, wherein the promoter proximal sequence comprises seven or more consecutive nucleotides.

Embodiment 7. The recombinant polynucleotide of any one of the preceding embodiments, wherein the promoter proximal sequence comprises eight or more consecutive nucleotides.

Embodiment 8. The recombinant polynucleotide of any one of the preceding embodiments, wherein the promoter proximal sequence comprises nine or more consecutive nucleotides.

Embodiment 9. The recombinant polynucleotide of any one of the preceding embodiments, wherein the promoter proximal sequence comprises ten or more consecutive nucleotides.

Embodiment 10. The recombinant polynucleotide of any one of the preceding embodiments, wherein the promoter proximal sequence comprises eleven or more consecutive nucleotides.

Embodiment 11. The recombinant polynucleotide of any one of the preceding embodiments, wherein the promoter proximal sequence comprises twelve or more consecutive nucleotides.

Embodiment 12. The recombinant polynucleotide of any one of the preceding embodiments, wherein the promoter proximal sequence comprises 2, 3, 4, 5, 6, 7, 8, 9, 10, 11 or 12 consecutive nucleotides.

Embodiment 13. The recombinant polynucleotide of any one of the preceding embodiments, wherein the promoter proximal sequence comprises 3 consecutive nucleotides.

Embodiment 14. The recombinant polynucleotide of any one of the preceding embodiments, wherein the promoter proximal sequence comprises 6 consecutive nucleotides.

Embodiment 15. The recombinant polynucleotide of any one of the preceding embodiments, wherein the promoter proximal sequence comprises 12 consecutive nucleotides.

Embodiment 16. The recombinant polynucleotide of any one of the preceding embodiments, wherein each of the consecutive nucleotides form exactly two hydrogen bonds when paired with a complementary nucleotide.

Embodiment 17. The recombinant polynucleotide of any one of embodiments 1-15, wherein each of the consecutive nucleotides forms exactly three hydrogen bonds when paired with a complementary nucleotide.

Embodiment 18. The recombinant polynucleotide of any one of the preceding embodiments, wherein the consecutive nucleotides are the same nucleotide.

Embodiment 19. The recombinant polynucleotide of any one of the preceding embodiments, wherein when paired with a complementary sequence of nucleotides, the promoter proximal sequence has a lower melting temperature than a reference promoter proximal sequence.

Embodiment 20. The recombinant polynucleotide of any one of the preceding embodiments, wherein the recombinant polynucleotide has a lower persistence length than a comparable reference polynucleotide, wherein the reference polynucleotide is identical to the recombinant polynucleotide except that the reference polynucleotide comprises a reference promoter proximal sequence instead of the promoter proximal sequence.

Embodiment 21. The recombinant polynucleotide of embodiment 19 or 20, wherein the reference promoter proximal sequence comprises the same number of nucleotides compared to the promoter proximal sequence.

Embodiment 22. The recombinant polynucleotide of embodiment 19 or 20, wherein the reference promoter proximal sequence comprises a fewer number of nucleotides compared to the promoter proximal sequence.

Embodiment 23. The recombinant polynucleotide of any one of embodiments 19-22, wherein the ratio of guanine/cytosine to adenine/thymine in the reference promoter proximal sequence is 1:1 or greater.

Embodiment 24. The recombinant polynucleotide of any one of the preceding embodiments, wherein the two or more consecutive nucleotides in the promoter proximal sequence are: adenine or a non-natural variant thereof, thymine or a non-natural variant thereof, cytosine or a non-natural variant thereof, or combinations thereof.

Embodiment 25. The recombinant polynucleotide of any one of embodiments 19-23, wherein the comparable reference promoter proximal sequence comprises only guanine.

Embodiment 26. The recombinant polynucleotide of any one of embodiments 19-23, wherein the comparable reference promoter proximal sequence comprises a lesser number of consecutive: adenine or a non-natural variant thereof, thymine or a non-natural variant thereof, or cytosine or a non-natural variant thereof, as compared to a promoter proximal sequence.

Embodiment 27. The recombinant polynucleotide of any one of the preceding embodiments, wherein the promoter proximal sequence comprises adenines, thymines, cytosines, or any combination thereof.

Embodiment 28. The recombinant polynucleotide of any one of the preceding embodiments, wherein the promoter proximal sequence is at least 3 nucleotides in length.

Embodiment 29. The recombinant polynucleotide of any one of the preceding embodiments, wherein the promoter proximal sequence is at least 4 nucleotides in length.

Embodiment 30. The recombinant polynucleotide of any one of the preceding embodiments, wherein the promoter proximal sequence is at least 5 nucleotides in length.

Embodiment 31. The recombinant polynucleotide of any one of the preceding embodiments, wherein the promoter proximal sequence is at least 6 nucleotides in length.

Embodiment 32. The recombinant polynucleotide of any one of the preceding embodiments, wherein the promoter proximal sequence is at least 7 nucleotides in length.

Embodiment 33. The recombinant polynucleotide of any one of the preceding embodiments, wherein the promoter proximal sequence is at least 8 nucleotides in length.

Embodiment 34. The recombinant polynucleotide of any one of the preceding embodiments, wherein the promoter proximal sequence is at least 9 nucleotides in length.

Embodiment 35. The recombinant polynucleotide of any one of the preceding embodiments, wherein the promoter proximal sequence is at least 10 nucleotides in length.

Embodiment 36. The recombinant polynucleotide of any one of the preceding embodiments, wherein the promoter proximal sequence is at least 11 nucleotides in length.

Embodiment 37. The recombinant polynucleotide of any one of the preceding embodiments, wherein the promoter proximal sequence is at least 12 nucleotides in length.

Embodiment 38. The recombinant polynucleotide of any one of the preceding embodiments, wherein the promoter proximal sequence is no more than 500 nucleotides in length.

Embodiment 39. The recombinant polynucleotide of any one of embodiments 1-27, wherein the promoter proximal sequence is about 3 nucleotides to about 500, about 3 to about 400, about 3 to about 300, about 3 to about 200, about 3 to about 100, about 3 to about 50, about 3 to about 40, about 3 to about 30, about 3 to about 20, about 3 to about 15, about 3 to about 14, about 3 to about 13, about 3 to about 12, about 3 to about 12, about 3 to about 11, about 3 to about 10, about 3 to about 9, about 3 to about 8, about 3 to about 7, about 3 to about 6, about 3 to about 5 nucleotides in length.

Embodiment 40. The recombinant polynucleotide of any one of embodiments 1-27, wherein the promoter proximal sequence is about 4 to about 500, about 5 to about 500, about 10 to about 500, about 20 to about 500, about 50 to about 500, about 100 to about 500, about 200 to about 500, about 300 to about 500 or about 400 to about 500 nucleotides in length.

Embodiment 41. The recombinant polynucleotide of any one of embodiments 1-27, wherein the promoter proximal sequence is at least about 3 nucleotides, at least about 4 nucleotides, at least about 5 nucleotides, at least about 6 nucleotides, at least about 7 nucleotides, at least about 8 nucleotides, at least about 9 nucleotides, at least about 10 nucleotides, at least about 11 nucleotides, at least about 12 nucleotides, at least about 13 nucleotides, at least about 14 nucleotides, at least about 15 nucleotides, at least about 16 nucleotides, at least about 17 nucleotides, at least about 18 nucleotides, at least about 19 nucleotides, or at least about 20 nucleotides in length.

Embodiment 42. The recombinant polynucleotide of any one of the preceding embodiments, wherein the promoter proximal sequence comprises adenines.

Embodiment 43. The recombinant polynucleotide of embodiment 42, wherein the promoter proximal sequence comprises at least 3 adenines, at least 4 adenines, at least 5 adenines, at least 6 adenines, at least 7 adenines, at least 8 adenines, at least 9 adenines, at least 10 adenines, at least 11 adenines, at least 12 adenines, at least 13 adenines, at least 14 adenines, or at least 15 adenines.

Embodiment 44. The recombinant polynucleotide of any one of embodiments 1-41, wherein the promoter proximal sequence comprises thymines.

Embodiment 45. The recombinant polynucleotide of embodiment 44, wherein the promoter proximal sequence comprises at least 3 thymines, at least 4 thymines, at least 5 thymines, at least 6 thymines, at least 7 thymines, at least 8 thymines, at least 9 thymines, at least 10 thymines, at least 11 thymines, at least 12 thymines, at least 13 thymines, at least 14 thymines, or at least 15 thymines.

Embodiment 46. The recombinant polynucleotide of any one of embodiments 1-41, wherein the promoter proximal sequence comprises cytosines.

Embodiment 47. The recombinant polynucleotide of embodiment 46, wherein the promoter proximal sequence comprises at least 3 cytosines, at least 4 cytosines, at least 5 cytosines, at least 6 cytosines, at least 7 cytosines, at least 8 cytosines, at least 9 cytosines, at least 10 cytosines, at least 11 cytosines, at least 12 cytosines, at least 13 cytosines, at least 14 cytosines, or at least 15 cytosines.

Embodiment 48. The recombinant polynucleotide of any one of the preceding embodiments, wherein the promoter is an RNA polymerase promoter.

Embodiment 49. The recombinant polynucleotide of embodiment 48, wherein the promoter is a bacteriophage promoter, a viral promoter, a bacterial promoter, a eukaryotic promoter or an engineered promoter.

Embodiment 50 The recombinant polynucleotide of embodiment 49, wherein the promoter is a T7 promoter or a variant or a fragment thereof.

Embodiment 51. The recombinant polynucleotide of embodiment 50, wherein the T7 promoter comprises an AGG sequence downstream of a TATA sequence, or a GGG downstream of a TATA sequence.

Embodiment 52. The recombinant polynucleotide of embodiment 50 or 51, wherein the promoter comprises a sequence of SEQ ID NO: 6 or SEQ ID NO: 70.

Embodiment 53. The recombinant polynucleotide of any one of the preceding embodiments, wherein the polynucleotide comprises a sequence of SEQ ID NO: 7, SEQ ID NO: 8, SEQ ID NO: 9 or SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO:79 or SEQ ID NO: 80.

Embodiment 54. The recombinant polynucleotide of embodiment 49, wherein the promoter is, a T3 promoter or a variant or a fragment thereof.

Embodiment 55. The recombinant polynucleotide of embodiment 54, wherein the T3 promoter comprises a sequence of SEQ ID NO: 17 or SEQ ID NO: 81.

Embodiment 56. The recombinant polynucleotide of any one of embodiments 1-49 or 54-55, wherein the polynucleotide comprises a sequence of SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89 or SEQ ID NO: 90.

Embodiment 57. The recombinant polynucleotide of embodiment 49, wherein the promoter is, or an SP6 promoter or a variant or a fragment thereof.

Embodiment 58. The recombinant polynucleotide of embodiment 57, wherein the SP6 promoter comprises a sequence of SEQ ID NO: 27.

Embodiment 59. The recombinant polynucleotide of any one of embodiments 1-49 or 57-58, wherein the polynucleotide comprises a sequence of SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35 or SEQ ID NO: 36.

Embodiment 60. The recombinant polynucleotide of any one of the preceding embodiments, wherein the sequence encoding the target is situated 3′ of the promoter proximal sequence.

Embodiment 61. The recombinant polynucleotide of any one of the preceding embodiments, wherein the target comprises a polyribonucleotide, e.g., an RNA oligo; a messenger RNA; a gRNA; an inhibitory RNA; an miRNA or siRNA; an antisense oligonucleotide; a long-non-coding RNA, a circular RNA, or any combination thereof.

Embodiment 62. The recombinant polynucleotide of any one of the preceding embodiments, further comprising one or more additional elements.

Embodiment 63. The recombinant polynucleotide of embodiment 62, wherein the one or more additional elements comprises one or more UTRs, a polyadenylation signal sequence, or combinations thereof.

Embodiment 64. A method making a polyribonucleotide, comprising a step of incubating a transcription mixture comprising:
(i) a recombinant polynucleotide of any one of the preceding embodiments;
(ii) at least one RNA polymerase that recognizes the promoter sequence; and
(iii) a plurality of ribonucleotides comprising at least two different types of ribonucleotides, each type comprising a different nucleoside;
   thereby producing the polyribonucleotide.

Embodiment 65. The method of embodiment 64, wherein the polyribonucleotide produced by the method is an in vitro transcribed polyribonucleotide.

Embodiment 66. The method of embodiment 64 or 65, wherein the in vitro transcription mixture further comprises a cap moiety to allow for co-transcriptional capping of the polyribonucleotide.

Embodiment 67. The method of embodiment 66, wherein the cap moiety comprises a Cap 1 structure.

Embodiment 68. The method of embodiment 66 or 67, wherein the cap moiety comprises m7G(5)ppp(5)(2′OmeA)Pg or a variant thereof.

Embodiment 69. The method of embodiment any one of embodiments 64-68, wherein the method produces a polyribonucleotide comprising a Cap1 structure that is co-transcriptionally added.

Embodiment 70. The method of any one of embodiments 64-69, wherein the polyribonucleotide is suitable for use as a therapeutic.

Embodiment 71. The method of embodiment 70, wherein the therapeutic is used in: (i) a method to stimulate an immune response; (ii) an antibody therapy method; (iii) an immune-modulation method; (iv) a vaccination method; (v) a gene therapy method; (vi) a cell therapy engineering method; (vii) an immunotherapy method; (viii) a protein replacement therapy method; (ix) a chemotherapeutic method; or (x) a combination of (i)-(ix).

Embodiment 72. The method of any one of embodiments 64-71, wherein the polyribonucleotide is in a composition.

Embodiment 73. The method of embodiment 72, wherein the composition is a pharmaceutical composition.

Embodiment 74. The method of any one of embodiments 64-73, wherein the polyribonucleotide is produced at a higher yield compared to an otherwise similar polyribonucleotide made with a DNA template without a promoter proximal sequence or with a different promoter proximal sequence.

Embodiment 75. The method of embodiment 74, wherein the yield is at least about 1.5-fold higher, at least about 2-fold higher, at least about 2.5-fold higher, at least about 3-fold higher, at least about 4-fold higher, at least about 5-fold higher, at least about 6-fold higher, at least about 7-fold higher, at least about 8-fold higher, at least about 9-fold higher, at least about 10-fold higher, at least about 20-fold higher, or at least about 50-fold higher.

Embodiment 76. The method of embodiment 74, wherein the yield is at least 1.5-fold higher, at least 2-fold higher, at least 2.5-fold higher, at least 3-fold higher, at least 4-fold higher, at least 5-fold higher, at least 6-fold higher, at least 7-fold higher, at least 8-fold higher, at least 9-fold higher, at least 10-fold higher, at least 20-fold higher, or at least 50-fold higher.

Embodiment 77. The method of any one of embodiments 64-76, wherein the polyribonucleotide is characterized in that when administered to a cell, tissue, or subject, the polyribonucleotide is expressed and/or translated at a substantially similar level when compared to expression and/or translation of an otherwise similar polyribonucleotide made with a DNA template without a promoter proximal sequence or with a different promoter proximal sequence that is administered to the cell, tissue, or subject.

Embodiment 78. The method of any one of embodiments 64-77, wherein the polyribonucleotide is characterized in that when administered to a cell, tissue, or subject, the cell, tissue, or subject has a substantially similar viability as compared to the viability of a cell, tissue, or subject administered an otherwise similar polyribonucleotide made with a DNA template without a promoter proximal sequence or with a different promoter proximal sequence.

Embodiment 79. The method of any one of embodiments 64-78, wherein the polyribonucleotide is characterized in that when administered to a cell, tissue, or subject, the polyribonucleotide has similar immunogenicity when compared to immunogenicity of an otherwise similar polyribonucleotide made with a DNA template without a promoter proximal sequence or with a different promoter proximal sequence that is administered to the cell, tissue, or subject.

Embodiment 80. The method of any one of embodiments 64-78, wherein the polyribonucleotide is characterized in that when administered to a cell, tissue, or subject, the polyribonucleotide has reduced immunogenicity when compared to immunogenicity of an otherwise similar polyribonucleotide made with a DNA template without a promoter proximal sequence or with a different promoter proximal sequence that is administered to the cell, tissue, or subject.

Embodiment 81. The method of embodiment 80, wherein reduced immunogenicity is observed with a polyribonucleotide comprising a promoter proximal sequence having less than 12 consecutive adenine, thymine or cytosine bases.

Embodiment 82. The method of embodiment 80 or 81, wherein the polyribonucleotide comprises a 2′O ribose modification.

Embodiment 83. The method of embodiment 80, wherein reduced immunogenicity is observed with a polyribonucleotide comprising a promoter proximal sequence having a modified ribonucleotide and at least six consecutive adenine, thymine or cytosine bases.

Embodiment 84. The method of embodiment 83, wherein the modified ribonucleotide is N4-acetylctyidine or 5-hydroxymethyluridine.

Embodiment 85. The method of any one of embodiments 80-84, wherein the polyribonucleotide further comprises a 2′O ribose modification.

Embodiment 86. The method of any one of embodiments 79-85, wherein immunogenicity is assessed by activation of pathways of NFkb, IRF, and/or other cytokines resulting from inflammation in the cell, tissue or organism.

Embodiment 87. The method of any one of embodiments 64-86, wherein the polyribonucleotide comprises one or more nucleosides comprising a modified nucleobases.

Embodiment 88. The method of embodiment 87, wherein the nucleobase comprising a modification is chosen from adenine, guanine, cytosine, or uracil.

Embodiment 89. The method of embodiment 88, wherein the modification comprises N4-acetyl-cytidine (ac4C), 5-hydroxymethyluridine (5-hmU), N1-methylpseudouridine, pyridin-4-one ribonucleoside, 5-aza-uridine, 6-aza-uridine, 2-thio-5-aza-uridine, 2-thio-uridine (s2U), 5-methyl cytidine (m5C), 5-aza-cytidine, 6-aza-cytidine, pseudoisocytidine, 3-methyl-cytidine (m3C), 5-formyl-cytidine (f5C), N4-methyl-cytidine (m4C), 2-amino-purine, 2, 6-diaminopurine, 2-amino-6-halo-purine, 6-halo-purine, inosine (I), 1-methyl-inosine (m1 I), wyosine (imG), methylwyosine (mimG), or any combination thereof.

Embodiment 90. The method of any one of embodiments 87-89, wherein the polyribonucleotide comprises N4-acetyl-cytidine (ac4C).

Embodiment 91. The method of embodiment 90, wherein the polyribonucleotide comprises cytidine residues and at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% of cytidine residues in the polyribonucleotide comprise N4-acetylcytidine.

Embodiment 92. The method of any one of embodiments 87-91, wherein the polyribonucleotide comprises 5-hydroxymethyluridine (5-hmU).

Embodiment 93. The method of embodiment 92, wherein the polyribonucleotide comprises uridine residues and at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% of 5-hydroxymethyluridine (5-hmU).

Embodiment 94. The method of any one of embodiments 87-93, wherein the polyribonucleotide comprises N4-acetyl-cytidine (ac4C) and 5-hydroxymethyluridine (5-hmU).

Embodiment 95. The method of embodiment 94, wherein:
(i) the polyribonucleotide comprises cytidine residues and at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% of cytidine residues in the polyribonucleotide comprise N4-acetylcytidine; and
(ii) the polyribonucleotide comprises uridine residues and at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% of 5-hydroxymethyluridine (5-hmU).

Embodiment 96. The method of any one of embodiments 87-95, wherein the polyribonucleotide comprises N1-methylpseudouridine,

Embodiment 97. The method of embodiment 96, wherein the polyribonucleotide comprises uridine residues and at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% of uridine residues in the polyribonucleotide comprise N1-methylpseudouridine.

Embodiment 98. The method of any one of embodiments 64-97, wherein the polyribonucleotide comprises one or more nucleosides comprising a modified ribose.

Embodiment 99. The method of embodiment 98, wherein the modified ribose is 2′-O-acetylated.

Embodiment 100. The method of embodiment 99, wherein: (i) at least 1% of the nucleosides comprise a 2-0 acetylated ribose; or (ii) no more than 90% of the nucleosides comprise a 2-0 acetylated ribose.

Embodiment 101. A polyribonucleotide made according to the method of any one of embodiments 64-100.

Embodiment 102. The polyribonucleotide of embodiment 101, wherein the polyribonucleotide is in a composition.

Embodiment 103. The polyribonucleotide of embodiment 102, wherein the composition is a pharmaceutical composition.

Embodiment 104. A pharmaceutical composition comprising a polyribonucleotide made according to the method of any one of embodiments 64-100.

Embodiment 105. A method comprising administering: a pharmaceutical composition made according to the method of any one of embodiments 64-100.

Embodiment 106. A method comprising administering the pharmaceutical composition of embodiment 104.

Embodiment 107. The method of embodiment 105 or 106, wherein the method is: (i) a method to stimulate an immune response; (ii) an antibody therapy method; (iii) an immune-modulation method; (iv) a vaccination method; (v) a gene therapy method; (vi) a cell therapy engineering method; (vii) an immunotherapy method; (viii) a protein replacement therapy method; (ix) a chemotherapeutic method; or (x) any combination of (i)-(ix).

Embodiment 108. Use of a polyribonucleotide made according to the method of any one of embodiments 64-100, in the preparation of a medicament for: (i) a method to stimulate an immune response; (ii) an antibody therapy method; (iii) an immune-modulation method; (iv) a vaccination method; (v) a gene therapy method; (vi) a cell therapy engineering method; (vii) an immunotherapy method; (viii) a protein replacement therapy method; (ix) a chemotherapeutic method; or (x) any combination of (i)-(ix).

Embodiment 109. Use of the pharmaceutical composition of embodiment 104, in the preparation of a medicament for: (i) a method to stimulate an immune response; (ii) an antibody therapy method; (iii) an immune-modulation method; (iv) a vaccination method; (v) a gene therapy method; (vi) a cell therapy engineering method; (vii) an immunotherapy method; (viii) a protein replacement therapy method; (ix) a chemotherapeutic method; or (x) any combination of (i)-(ix).

Embodiment 110. A composition comprising a polyribonucleotide made according to the method of any one of embodiments 64-100, for use in: (i) a method to stimulate an immune response; (ii) an antibody therapy method; (iii) an immune-modulation method; (iv) a vaccination method; (v) a gene therapy method; (vi) a cell therapy engineering method; (vii) an immunotherapy method; (viii) a protein replacement therapy method; (ix) a chemotherapeutic method; or (x) any combination of (i)-(ix).

Embodiment 111. A composition comprising the pharmaceutical composition of embodiment 104, for use in: (i) a method to stimulate an immune response; (ii) an antibody therapy method; (iii) an immune-modulation method; (iv) a vaccination method; (v) a gene therapy method; (vi) a cell therapy engineering method; (vii) an immunotherapy method; (viii) a protein replacement therapy method; (ix) a chemotherapeutic method; or (x) any combination of (i)-(ix).

Embodiment 112. The method of any one of embodiments 105-107, wherein the polyribonucleotide or the pharmaceutical composition is administered to a subject.

Embodiment 113. The use of embodiment 108 or 109, wherein the polyribonucleotide or the pharmaceutical composition is administered to a subject.

Embodiment 114. The composition for use of embodiment 110 or 111, wherein the composition is administered to a subject.

Embodiment 115. The method of embodiment 112, the use of embodiment 113 or the composition for use of embodiment 114, wherein the subject is a mammal.

Embodiment 116. The method of embodiment 115, the use of embodiment 115 or the composition for use of embodiment 115, wherein the mammal is a human.

### Exemplification

### Example 1: Materials and Methods

IVT Template production: The Luc2 gene encoding an optimized version of firefly luciferase was amplified from Pucidt-AMP (IDT). Amplification was carried out at an annealing temperature of 50° C. in a 20 I reaction consisting of 0.25 mM each primer, which were used to add three different lengths of poly-nucleotide fragments after the core sequence corresponding to the bacteriophage T7 promoter starting on the +4 position; as well as 1× Herculase II buffer, 25 mM each dNTP, 15 ng Pucidt-AMP (IDT) and 2 U Herculase II enzyme per PCR reaction. After incubation, the PCR reaction was subjected to treatment with 20 U of Dpn1 enzyme (New England Biolabs) to digest the template plasmid. The digested products were purified using the DNA Clean & Concentrator-25 kit (Zymo Research) and eluted into 30 μL nuclease free water. The Luc2 gene encoding an optimized version of firefly luciferase was amplified from Pucidt-AMP (IDT). Amplification was carried out at an annealing temperature of 50° C. in a 20 μL reaction consisting of 0.25 mM each primer, which were used to add three different lengths of 5′ poly-adenine, poly-thymine, poly-cytosine or poly-guanine tracts after the sequence corresponding to the bacteriophage T7 promoter.

After incubation, the PCR reaction was subjected to treatment with 20 U of Dpn1 enzyme (New England Biolabs) to digest template plasmid. The digested products were purified using the DNA Clean & Concentrator-25 kit (Zymo Research) and eluted into 30 μL nuclease free water. The sequences of primers used were as follows:

```
T7-AGG-fwd:
  (SEQ ID NO: 1)
gaattTAATACGACTCACTATAAGGcttgttctttttgcagaagc
T7-AGG-fwd + 3As:
  (SEQ ID NO: 2)
gaattTAATACGACTCACTATAAGGAAActtgttctttttgcagaagc
T7-AGG-fwd + 6As:
  (SEQ ID NO: 3)
gaattTAATACGACTCACTATAAGGAAAAAActtgttctttttgcagaagc
T7-AGG-fwd + 12As:
  (SEQ ID NO: 4)
gaattTAATACGACTCACTATAAGGAAAAAAAAAAAActtgttctttttgcagaagc
120Pa_rev:
  (SEQ ID NO: 5)
TTTTTTTTTTTTTTTTTTTTTTTTTTTTTTTTTTTTTTTTTTTTTTTTTTTTTTTTTTTTTTTTTTTTTTTTTTTTTTTTTTTTTTTT
TTTTTTTTTTTTTTTTTTTTTTTTTTTTTTTagaatgtgaagaaactttctttttattag
T7_AGG + 3Ts_fwd
  (SEQ ID NO: 37)
GAATTTAATACGACTCACTATAAGGTTTcttgttctttttgcagaagc
T7_AGG + 6Ts_fwd:
  (SEQ ID NO: 38)
GAATTTAATACGACTCACTATAAGGTTTTTTcttgttctttttgcagaagc
T7_AGG + 12Ts_fwd:
  (SEQ ID NO: 39)
GAATTTAATACGACTCACTATAAGGTTTTTTTTTTTTcttgttctttttgcagaagc
T7_AGG+ 3Cs_fwd:
  (SEQ ID NO: 40)
GAATTTAATACGACTCACTATAAGGCCCcttgttctttttgcagaagc
T7_AGG + 6Cs_fwd:
  (SEQ ID NO: 41)
GAATTTAATACGACTCACTATAAGGCCCCCCcttgttctttttgcagaagc
T7_AGG + 12Cs_fwd:
  (SEQ ID NO: 42)
GAATTTAATACGACTCACTATAAGGCCCCCCCCCCCCcttgttctttttgcagaagc
T7_AGG + 3Gs_fwd:
  (SEQ ID NO: 43)
GAATTTAATACGACTCACTATAAGGGGGcttgttctttttgcagaagc
T7_AGG + 6Gs_fwd:
(SEQ ID NO: 44)
GAATTTAATACGACTCACTATAAGGGGGGGGcttgttctttttgcagaagc
T7_AGG+ 12Gs_fwd:
(SEQ ID NO: 45)
GAATTTAATACGACTCACTATAAGGGGGGGGGGGGGGcttgttctttttgcagaagc
T3_no_Pa_fwd:
  (SEQ ID NO: 46)
AATTAACCCTCACTAAAAGGcttgttctttttgcagaagc
T3 + 3As_fwd:
  (SEQ ID NO: 47)
AATTAACCCTCACTAAAAGGAAActtgttctttttgcagaagc
T3 + 6As_fwd:
  (SEQ ID NO: 48)
AATTAACCCTCACTAAAAGGAAAAAActtgttctttttgcagaagc
T3 + 12As_fwd:
(SEQ ID NO: 49)
ATTAACCCTCACTAAAAGGAAAAAAAAAAAActtgttctttttgcagaagc
ASP6_no_Pa_AGG_fwd:
  (SEQ ID NO: 50)
ATTTAGGTGACACTATAAGGcttgttctttttgcagaagc
SP6 + 3As_AGG_fwd:
  (SEQ ID NO: 51)
ATTTAGGTGACACTATAAGGAAActtgttctttttgcagaagc
SP6 + 6As_AGG_fwd :
  (SEQ ID NO: 53)
ATTTAGGTGACACTATAAGGAAAAAActtgttctttttgcagaagc
SP6 + 12As_AGG_fwd:
  (SEQ ID NO: 54)
ATTTAGGTGACACTATAAGGAAAAAAAAAAAActtgttctttttgcagaagc
SP6_no_Pa_AGA_fwd:
  (SEQ ID NO: 55)
ATTTAGGTGACACTATAAGActtgttctttttgcagaagc
SP6 + 3As_AGA_fwd:
(SEQ ID NO: 56)
ATTTAGGTGACACTATAAGAAAActtgttctttttgcagaagc
SP6 + 6As_AGA_fwd:
  (SEQ ID NO: 57)
ATTTAGGTGACACTATAAGAAAAAAActtgttctttttgcagaagc
SP6 + 12As_AGA_fwd:
(SEQ ID NO: 58)
ATTTAGGTGACACTATAAGAAAAAAAAAAAAActtgttctttttgcagaagc
Luc2 gene encoding an optimized version of firefly luciferase amplified from Pucidt-AMP (IDT):
  (SEQ ID NO: 59)
CTTGTTCTTTTTGCAGAAGCTCAGAATAAACGCTCAACTTTGGCCACCATGGAAGATGCCAAAAACATTAAGAAGGGCCCAGCGC
CATTCTACCCACTCGAAGACGGGACCGCCGGCGAGCAGCTGCACAAAGCCATGAAGCGCTACGCCCTGGTGCCCGGCACCATCG
CCTTTACCGACGCACATATCGAGGTGGACATTACCTACGCCGAGTACTTCGAGATGAGCGTTCGGCTGGCAGAAGCTATGAAGC
GCTATGGGCTGAATACAAACCATCGGATCGTGGTGTGCAGCGAGAATAGCTTGCAGTTCTTCATGCCCGTGTTGGGTGCCCTGT
TCATCGGTGTGGCTGTGGCCCCAGCTAACGACATCTACAACGAGCGCGAGCTGCTGAACAGCATGGGCATCAGCCAGCCCACCG
TCGTATTCGTGAGCAAGAAAGGGCTGCAAAAGATCCTCAACGTGCAAAAGAAGCTACCGATCATACAAAAGATCATCATCATGGA
TAGCAAGACCGACTACCAGGGCTTCCAAAGCATGTACACCTTCGTGACTTCCCATTTGCCACCCGGCTTCAACGAGTACGACTTC
GTGCCCGAGAGCTTCGACCGGGACAAAACCATCGCCCTGATCATGAACAGTAGTGGCAGTACCGGATTGCCCAAGGGCGTAGCC
CTACCGCACCGCACCGCTTGTGTCCGATTCAGTCATGCCCGCGACCCCATCTTCGGCAACCAGATCATCCCCGACACCGCTATCC
TCAGCGTGGTGCCATTTCACCACGGCTTCGGCATGTTCACCACGCTGGGCTACTTGATCTGCGGCTTTCGGGTCGTGCTCATGT
ACCGCTTCGAGGAGGAGCTATTCTTGCGCAGCTTGCAAGACTATAAGATTCAATCTGCCCTGCTGGTGCCCACACTATTTAGCTT
CTTCGCTAAGAGCACTCTCATCGACAAGTACGACCTAAGCAACTTGCACGAGATCGCCAGCGGCGGGGCGCCGCTCAGCAAGGA
GGTAGGTGAGGCCGTGGCCAAACGCTTCCACCTACCAGGCATCCGCCAGGGCTACGGCCTGACAGAAACAACCAGCGCCATTCT
GATCACCCCCGAAGGGGACGACAAGCCTGGCGCAGTAGGCAAGGTGGTGCCCTTCTTCGAGGCTAAGGTGGTGGACTTGGACA
CCGGTAAGACACTGGGTGTGAACCAGCGCGGCGAGCTGTGCGTCCGTGGCCCCATGATCATGAGCGGCTACGTTAACAACCCC
GAGGCTACAAACGCTCTCATCGACAAGGACGGCTGGCTGCACAGCGGCGACATCGCCTACTGGGACGAGGACGAGCACTTCTTC
ATCGTGGACCGGCTGAAGAGCCTGATCAAATACAAGGGCTACCAGGTAGCCCCAGCCGAACTGGAGAGCATCCTGCTGCAACAC
CCCAACATCTTCGACGCCGGGGTCGCCGGCCTGCCCGACGACGATGCCGGCGAGCTGCCCGCCGCAGTCGTCGTGCTGGAACA
CGGTAAAACCATGACCGAGAAGGAGATCGTGGACTATGTGGCCAGCCAGGTTACAACCGCCAAGAAGCTGCGCGGTGGTGTTG
TGTTCGTGGACGAGGTGCCTAAAGGACTGACCGGCAAGTTGGACGCCCGCAAGATCCGCGAGATTCTCATTAAGGCCAAGAAGG
GCGGCAAGATCGCCGTGTAATAATGATAGACCAGCCTCAAGAACACCCGAATGGAGTCTCTAAGCTACATAATACCAACTTACAC
TTTACAAAATGTTGTCCCCCAAAATGTAGCCATTCGTATCTGCTCCTAATAAAAAGAAAGTTTCTTCACATTCT
```

In vitro transcription (IVT) of Luc2 RNA: The different Luc2 RNA constructs were synthesized in 20 μL IVT reactions consisting of 100 ng of the corresponding Luc2 template, 20 mM MgCl2, 7.5 mM each NTP, 1× HiScribe Transcription Buffer, and 2 μL HiScribe polymerase mix (New England Biolabs) and incubated at 37° C. for 2 hours.

The transcribed products were then digested in 24 μL reactions consisting of 2 U of Dnase I (Rnase-free) (New England Biolabs) and 20 U of Alkaline Phosphatase (Promega) at 37° C. for 7 minutes to degrade DNA template and non-capped 5′-triphosphate RNA. Dnase I treated samples were cleaned up using Monarch 500 ug RNA Clean Up kit (New England Biolabs) and eluted into 80 μL nuclease-free water.

RNA Quantification: RNA concentration was determined using a NanoDrop OneC spectrophotometer (Thermo Scientific).

2% Agarose denaturing gels: The RNA samples were diluted in a 1:20 ratio with water and loaded into a 2% E-Gel EX precast agarose gel (Invitrogen).

Cell lines: A549-Dual cells (InvivoGen) were cultured in high glucose GlutaMAX Dulbecco's Modified Eagle Medium (Thermo Fisher) supplemented with 10% heat-inactivated fetal bovine serum, 100 units/mL penicillin, 100 μg/mL streptomycin, 10 μg/mL blasticidin, and 0.1 μg/mL zeocin and maintained at 37° C. and 5% CO₂. Cells were plated in a 96-well plate at 2,000 cells/well 24 h prior to transfection.

Cell transfection: Transfection was carried out using Lipofectamine MessengerMAX (Thermo Fisher) at a 1.5 μL reagent per μg RNA and using Opti-MEM media as diluent.

Cell Readouts: Viability and expression levels were assayed using the One-Glo+Tox Luciferase Reporter and Cell Viability Assay kit (Promega) 72 h after transfection. Immunogenicity was measured at the same time point, where NF-Kb promoter activation was measured using the QUANTI-Blue assay (InvivoGen), and IRF reporter activation was measured using the QUANTI-Luc assay (Invivogen). All measurements were taken on a GloMax Discover Microplate Reader (Promega). The signal data from experimental groups was normalized to untreated cells.

Capillary electrophoresis: RNA was analyzed by capillary electrophoresis using a pre-assembled BFS capillary cartridge operated by the BioPhase 8800 system (SCIEX). Reagents provided on the RNA 9000 Purity & Integrity kit (SCIEX) were prepared according to manufacturer instructions and placed on disposable BioPhase starter outlet and inlet plates. RNA samples were prepared at a final concentration of 3 ng/μL with Nuclease free-water (New England Biolabs) and a subsequent 1:1 dilution was prepared with sample loading solution (SLS, SCIEX) for the final 60 μL working solution at a concentration of 1.5 ng/μL. Finally, samples were denatured at 70 C for 5 minutes and immediately placed on a PCR cold block for 2-5 minutes. Similarly, the ssRNA ladder was prepared as described in the user manual for the mentioned kit. Data acquisition and analysis were performed using the BioPhase 8800 software v1.2.20 e-license.

### Example 2: Use of Polynucleotides Having a T7 Bacteriophage Promoter with Varying Lengths of Promoter Proximal Sequences for Generating RNA

This Example describes use of exemplary DNA templates having poly-adenine, poly-cytosine, poly-thymine, or poly-guanine tracts at the 3′ end of a T7 bacteriophage promoter for synthesizing RNA. The methods used in this Example are provided in Example 1.

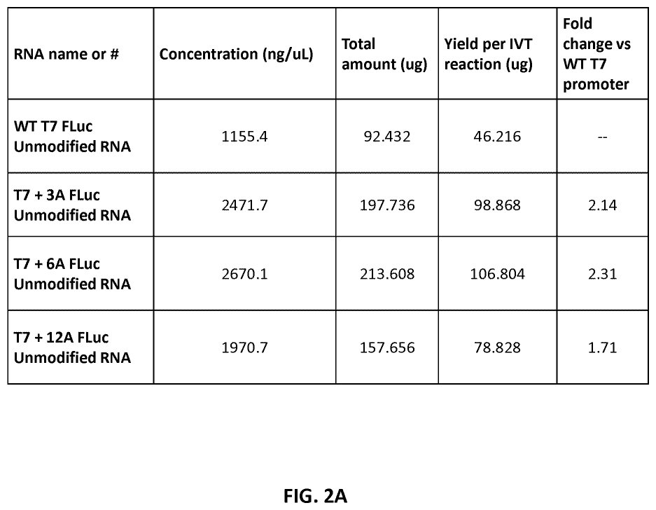

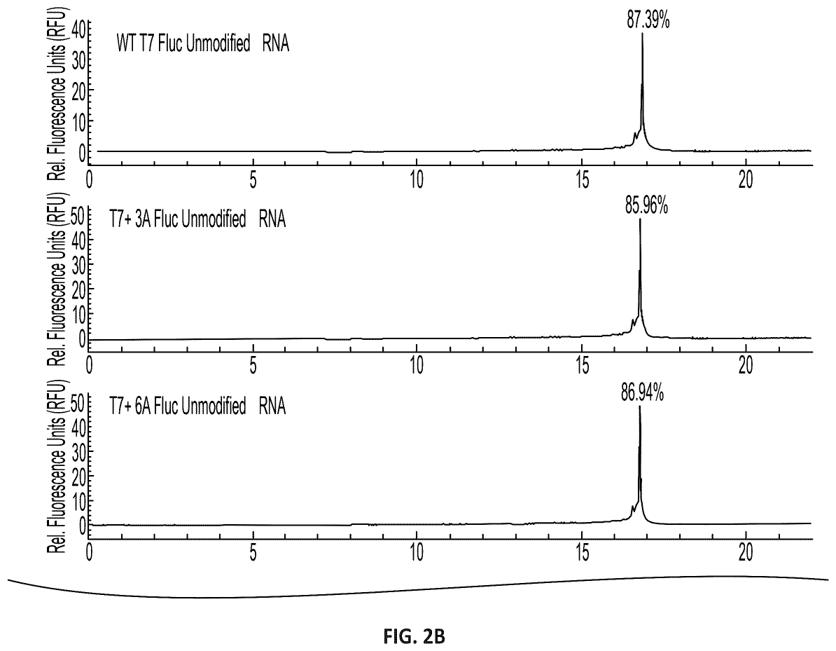

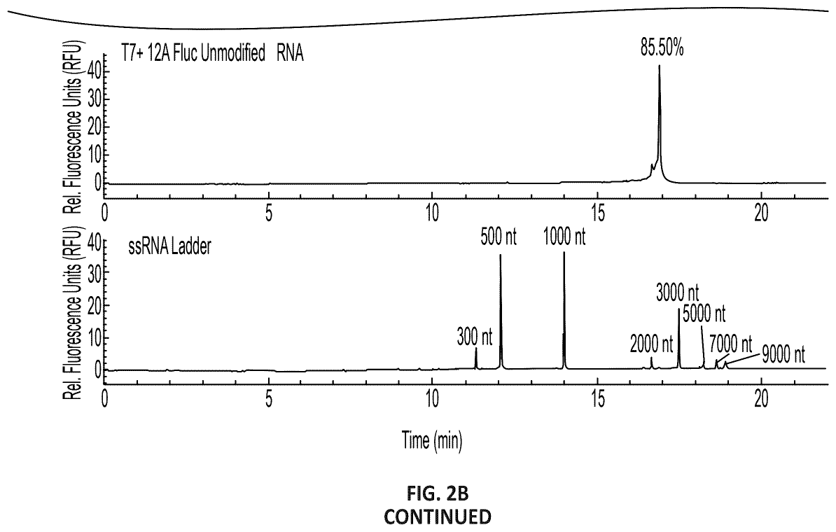

FIG. 2A, shows the increase on total unmodified Mrna yield after in vitro transcription reactions using four DNA templates encoding an exemplary payload (Fluc protein), three of which had increasing poly-adenine tracts downstream (i.e., at the 3′ end) of the T7 bacteriophage core promoter starting on the +4 position. By adding three and six extra adenines (T7+3A and T7+6A) yield of the exemplary RNA increased approximately 2-fold, and 2.3-fold, respectively, compared to RNA yield with the WT T7 promoter. In contrast, adding a 12-mer poly-adenine tract (T7+12A) increased yield of the exemplary RNA by on 1.7-fold. FIG. 2B shows the electropherogram of the different sample traces detected by gel capillary electrophoresis (CE) of the mentioned RNAs, along with an ssRNA ladder as a size reference. According to the CE analysis, all RNAs exhibited similar traces corresponding to the full-length desired product and non-tailed impurities. Additionally, the data shows that the purity of exemplary RNAs with increasing poly-adenine tracts (made with DNA templates having poly-adenine tracts at the 3′end of the T7 promoter) did not impact RNA purity as compared to RNA purity of RNA made from DNA templates having the WT T7 control (purity >85%).

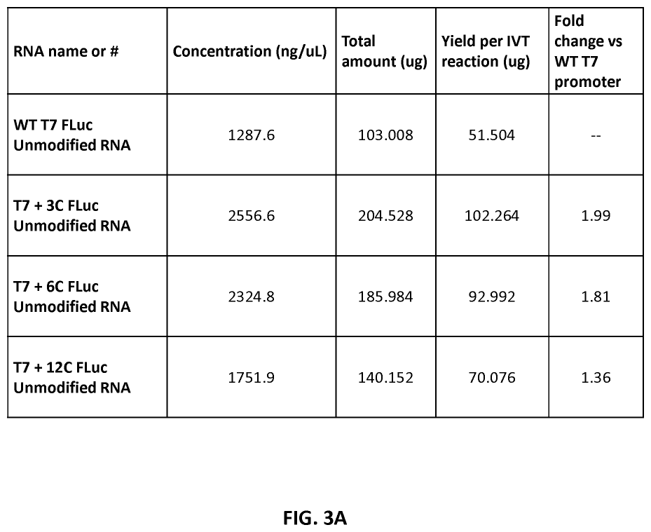

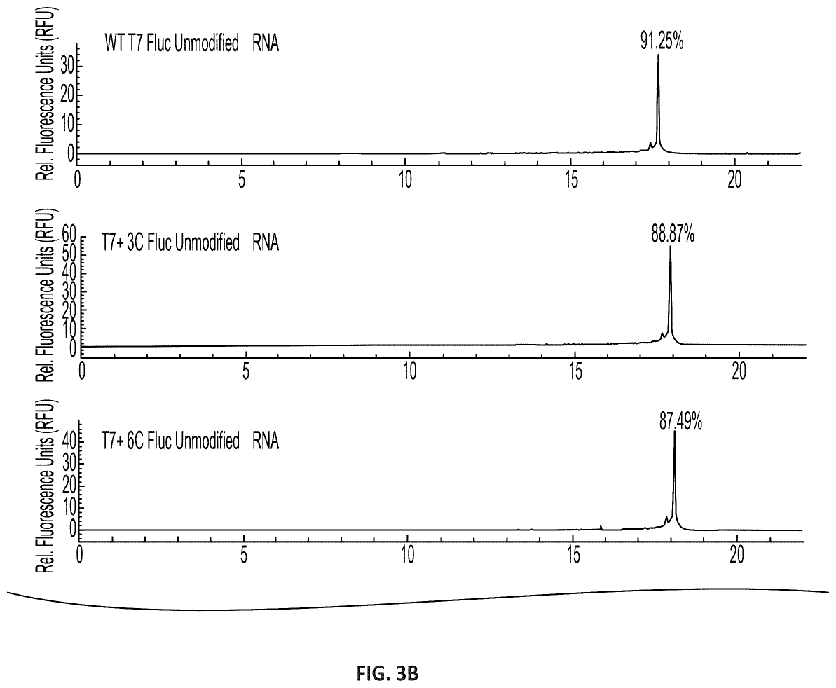

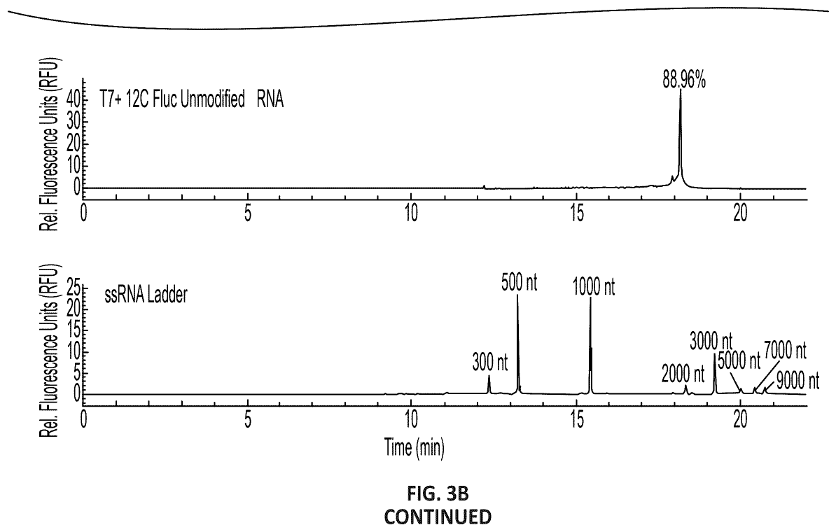

FIG. 3A shows total RNA yield after an in vitro transcription reaction using DNA templates encoding an exemplary payload (Fluc protein) with a wild-type T7 bacteriophage promoter, or with a T7 promoter having poly-cytosine tracts of increasing length at the 3′ end of the T7 promoter: 3 cytosines, 6 cytosines, or 12 cytosines. RNA was synthesized with unmodified ribonucleotides. By adding three or six cytosines (T7+3C or T7+6C), RNA yield increased approximately 2-fold, and 1.8-fold, respectively, compared to RNA yield with the WT T7 promoter. In comparison, when a 12-mer poly-cytosine tract was used (T7+12C) the yield was only 1.4-fold higher. According to CE analysis shown in FIG. 3B, each RNA exhibited similar traces corresponding to the desired product and non-tailed impurities. Additionally, the results indicate that the purity of RNAs having increasing poly-cytosine tracts (made with DNA templates having increasing poly-cytosine tracts) was comparable to RNA purity observed with RNA made from DNA templates having the WTT7 promoter (purity >85%).

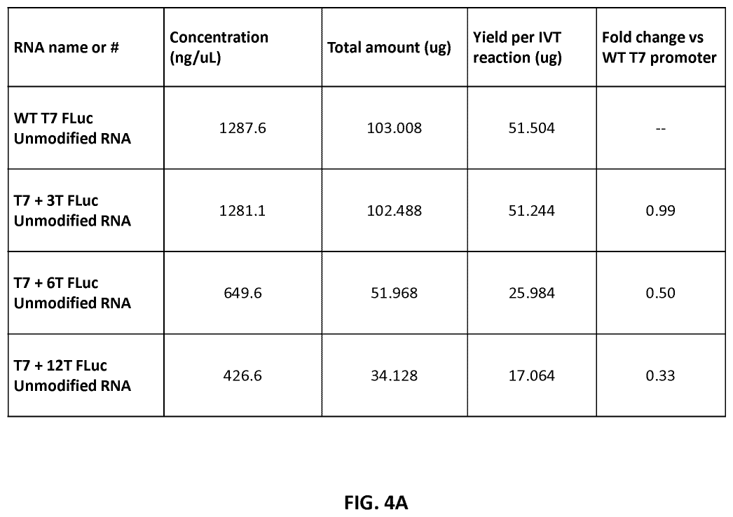

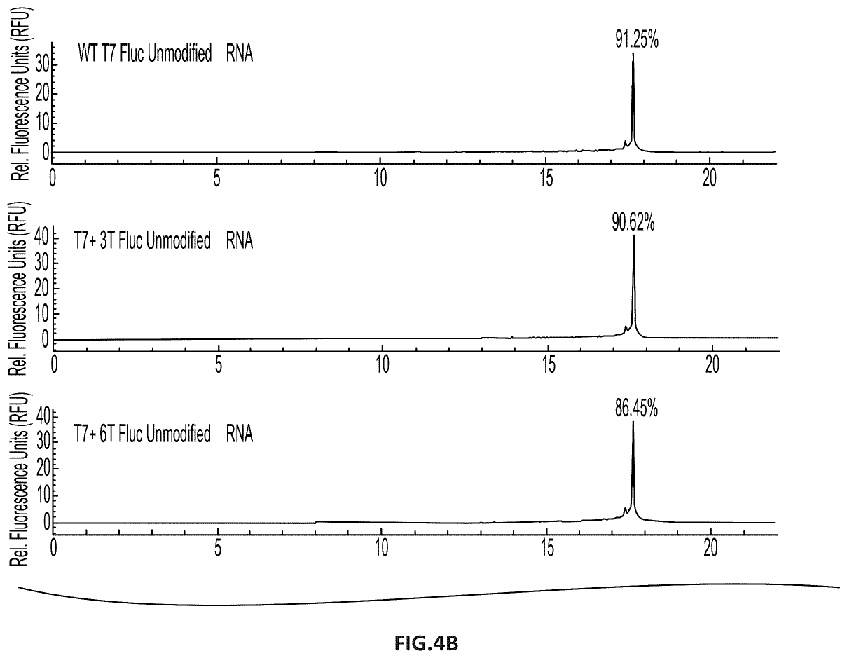

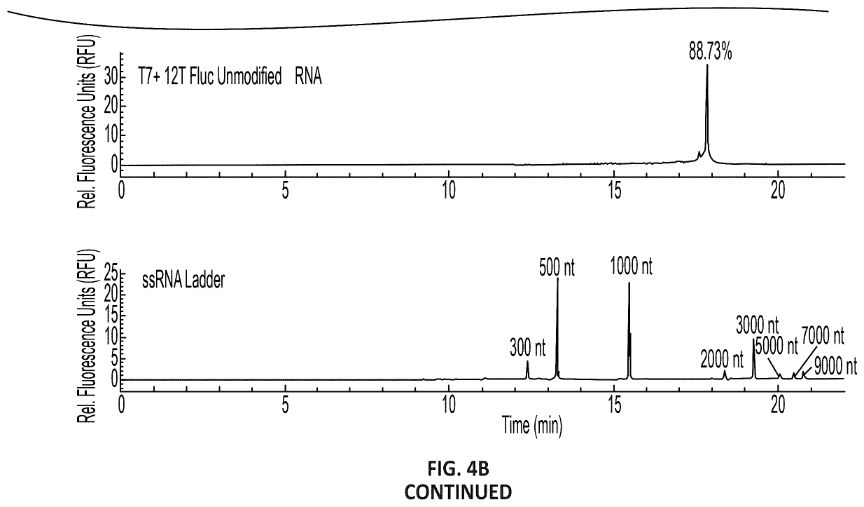

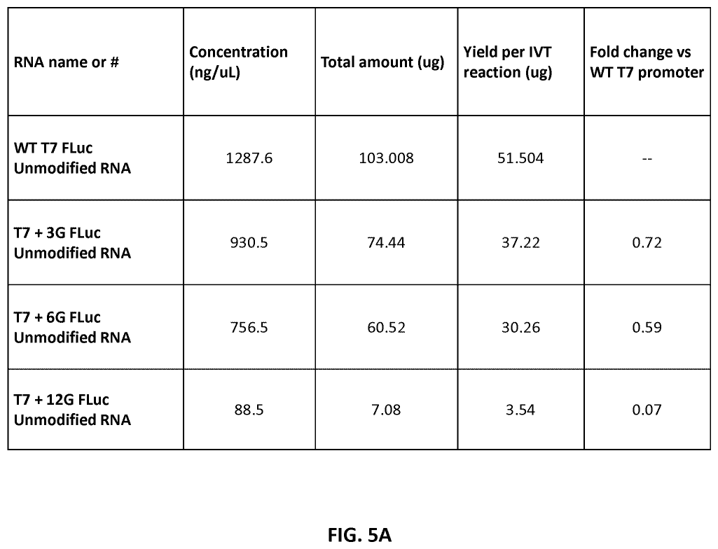

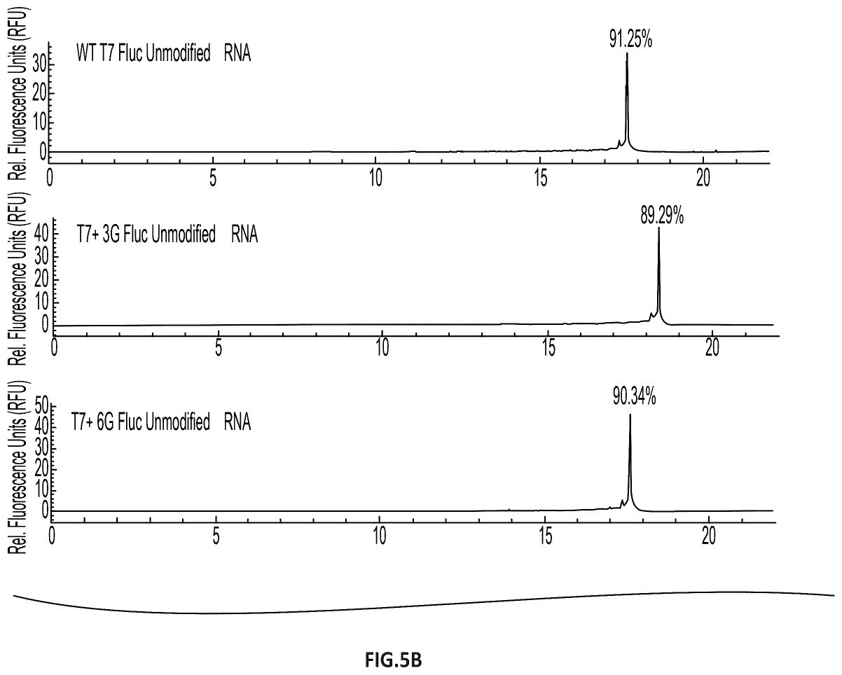

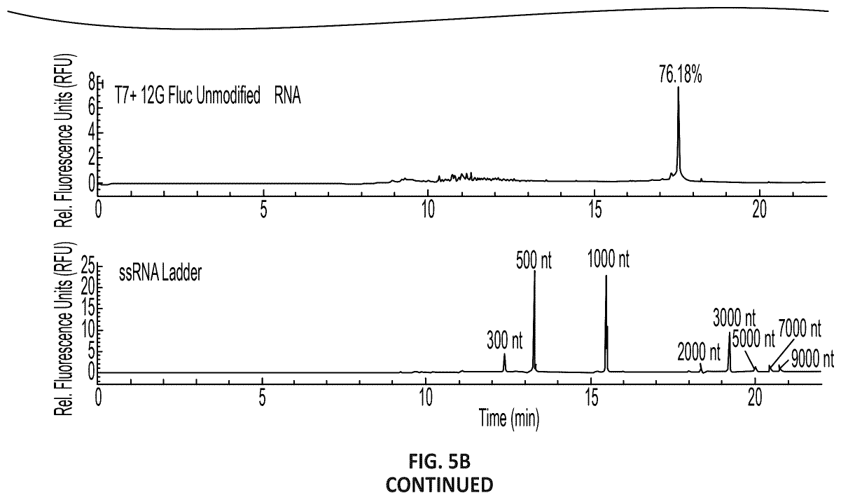

FIG. 4A shows total yield of RNA after an in vitro transcription reaction using DNA templates encoding an exemplary payload (Fluc protein) with a wild type T7 bacteriophage promoter, or a T7 promoter having poly-thymine tracts of increasing length at the 3′ end of the T7 promoter: 3 thymines, 6 thymines, or 12 thymines. RNA was synthesized with unmodified ribonucleotides. FIG. 5A shows total yield of RNA after an in vitro transcription reaction using DNA templates encoding an exemplary payload (Fluc protein) with a wild type T7 bacteriophage promoter, or a T7 promoter having poly-guanine tracts of increasing length at the 3′ end of the T7 promoter: 3 guanines, 6 guanines, or 12 guanines. RNA was synthesized with unmodified ribonucleotides.

In contrast to RNAs having poly-adenine or poly-cytosine tracts, addition of poly-thymine or poly-guanine at the 3′ end of the T7 bacteriophage promoter decreased total RNA yield. By adding three, six or twelve thymines to the DNA template, RNA yield reduced by approximately 0.99-fold, 0.50-fold and 0.33-fold, respectively, compared to RNA yield with a wild type T7 promoter (FIG. 4A). Moreover, by adding three, six, or twelve guanines, RNA yield reduced by approximately 0.72-fold, 0.59-fold and 0.07-fold, respectively, compared to the wild type T7 promoter (FIG. 5A).

As shown in FIG. 4B, poly-uridine bearing RNAs (transcribed from DNA templates having poly-uridine tracts at the 3′ end of the T7 promoter) had a similar purity profile as compared to poly-adenine and poly-cytosine bearing RNAs (purity >85%). In contrast to poly-adenine, poly-cytosine and poly-uridine containing RNAs, only the addition of 3- and 6-mer poly-guanine at the 3′ end of the T7 promoter in the template DNA, generated full-length RNAs as the main product. Addition of a 12-mer poly-guanine generated several truncated RNA products, as reflected on the earlier migration times of traces and significantly decreased the purity of the RNAs (<80%) (FIG. 5B).

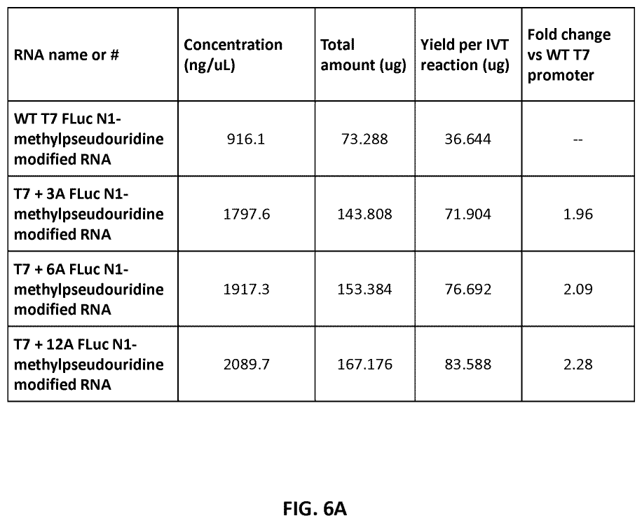

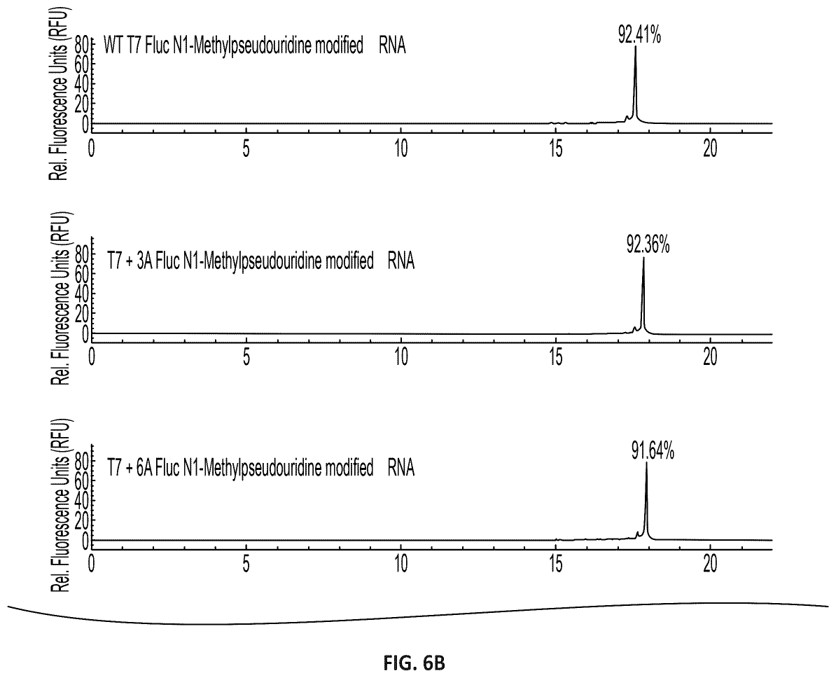

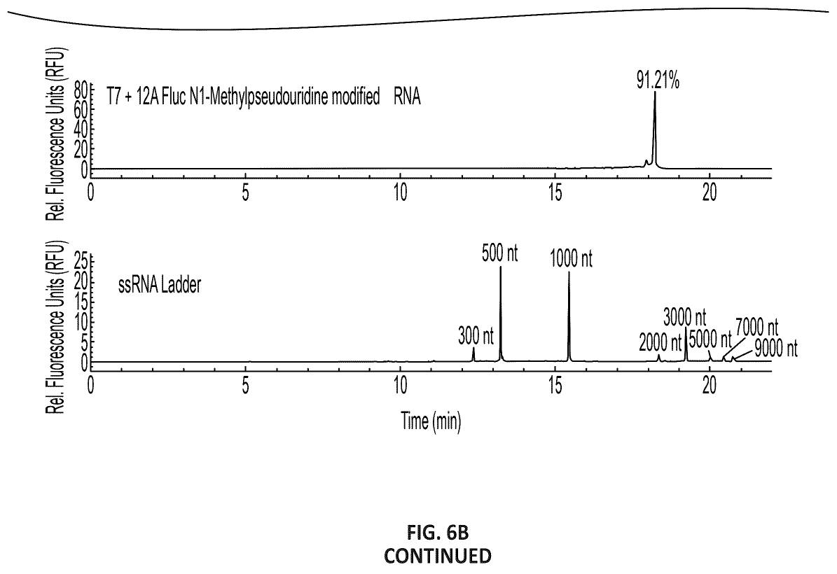

Next, the impact of synthesizing polyribonucleotides comprising modified nucleotides (e.g., N1-methylpseudouridine, Ac4c, and/or 5hmU) with DNA templates having poly-adenine, poly-cytosine, poly-thymine, or poly-guanine tracts of varying lengths at the 3′ end of a T7 promoter was analyzed. N1-methylpseudouridine modified RNA was synthesized using DNA templates described above. By adding three, six, or twelve adenines, N1-methylpseudouridine modified RNA yield increased 1.96-fold, 2.09-fold and 2.28-fold, respectively, compared to RNA yield with the wild type T7 promoter (FIG. 6A). N1-methylpseudouridine modified RNA having increased poly-adenine tracts (made with DNA having increased poly-adenine tracts) displayed a purity of >90% with similar trace profiles as observed with RNA made with DNA having the WT T7 promoter (FIG. 6B).

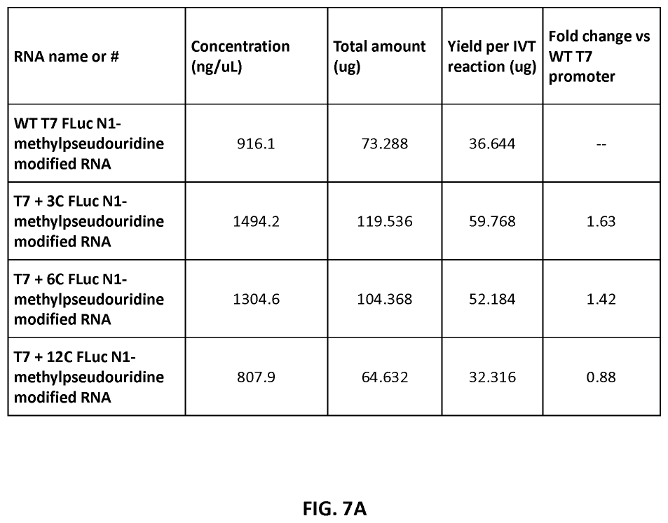

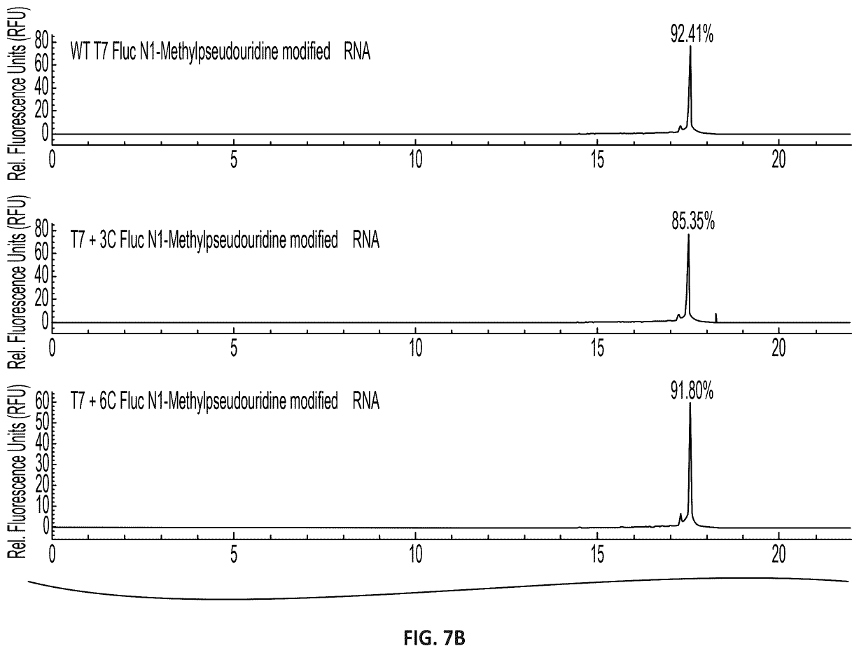

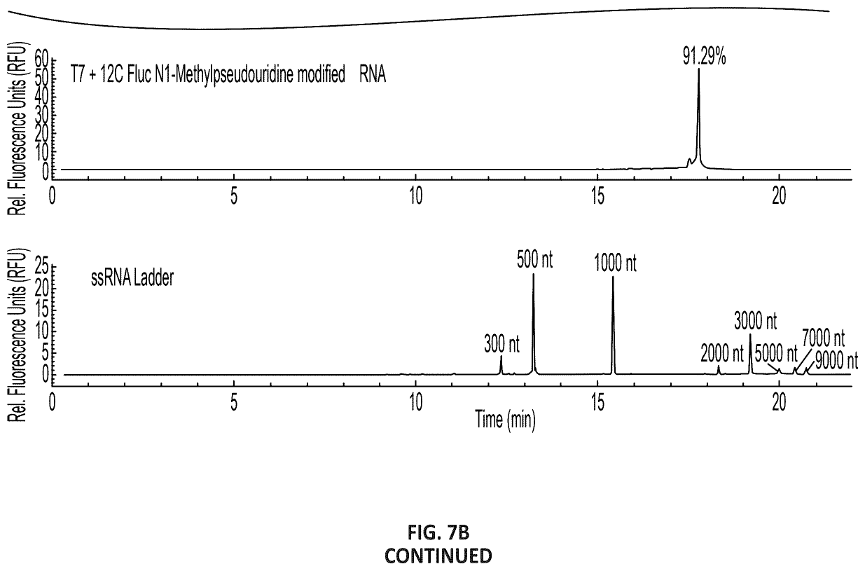

The effect on RNA yield with N1-methylpseudouridine RNA synthesized from DNA templates with increasing poly-cytosine tracts, was also similar to the unmodified RNAs. By adding three, or six cytosines, RNA yield increased by 1.63-fold, and 1.4-fold, respectively. Addition of twelve cytosines decreased RNA yield by 0.88-fold, compared to yield of RNA made with a DNA template having the wild type T7 promoter (FIG. 7A). N1-methylpseudouridine modified RNA having increased poly-cytosine tracts (made with DNA having increased poly-cytosine tracts) displayed a purity of >85% with similar trace profiles as observed with RNA made with DNA having the WT T7 promoter (FIG. 7B).

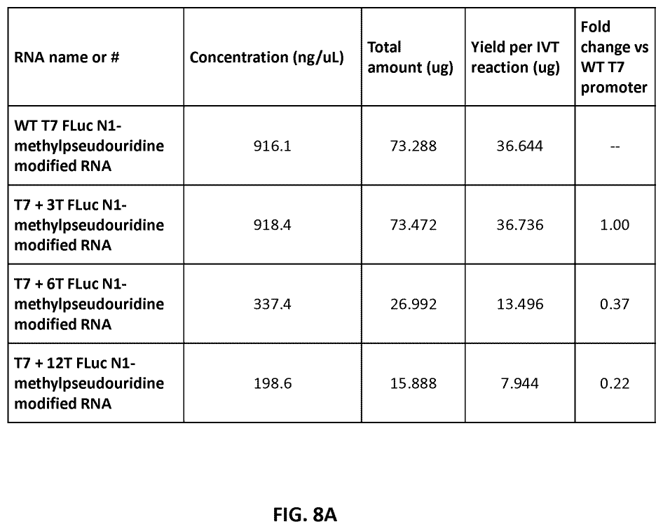

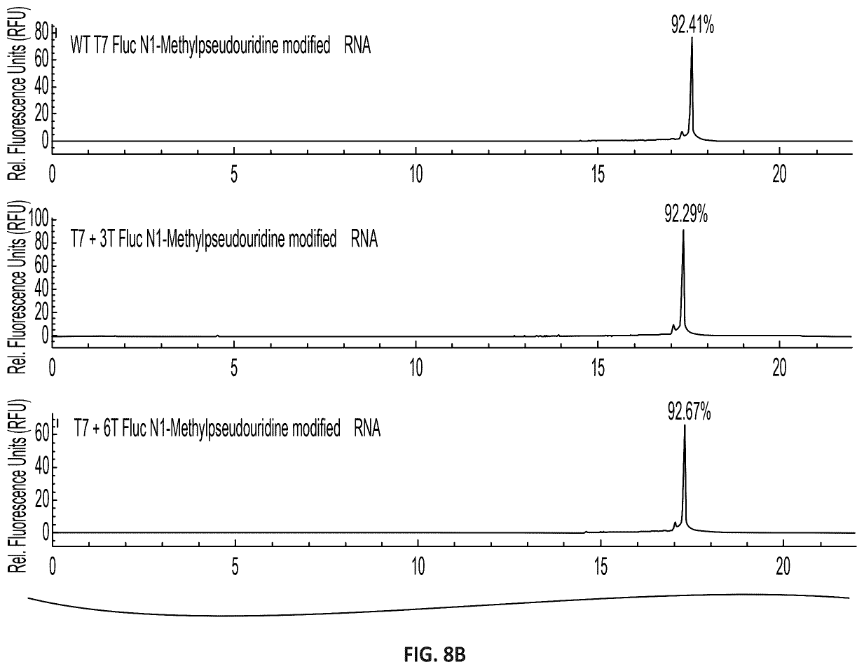

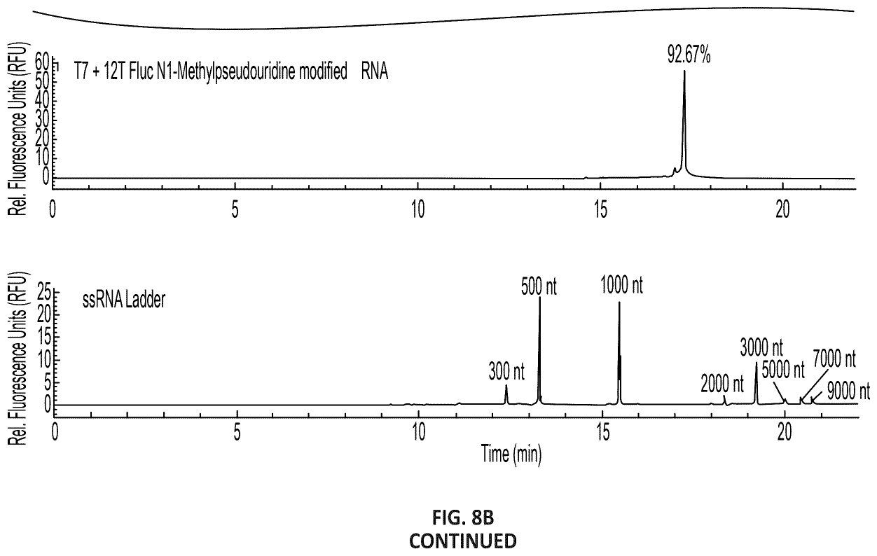

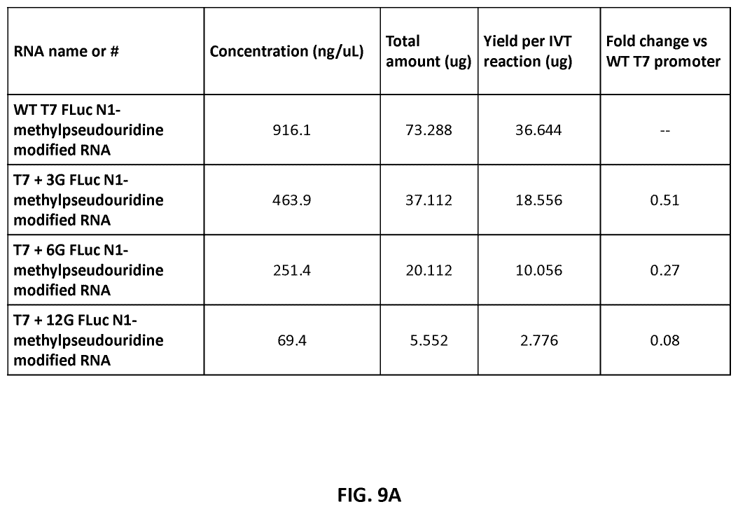

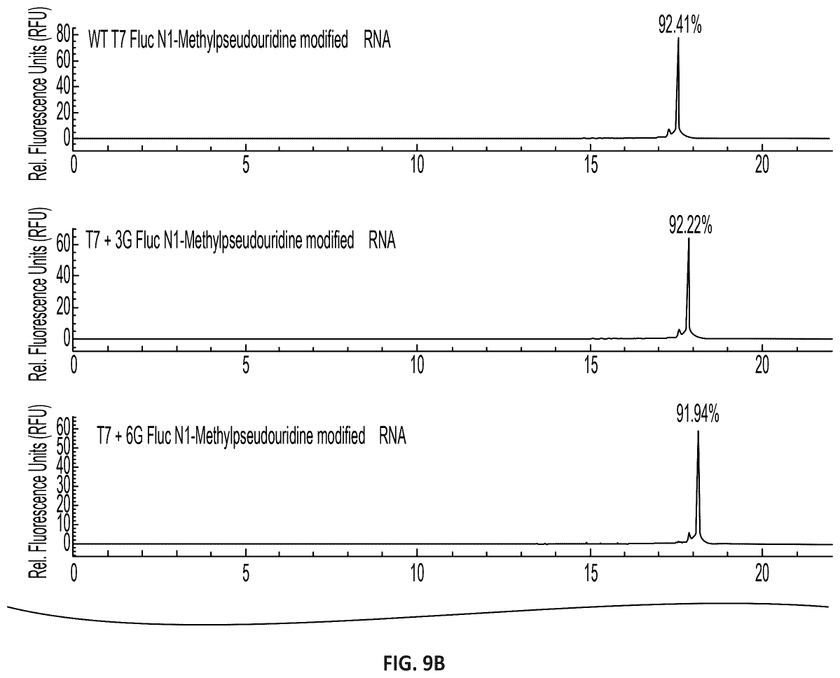

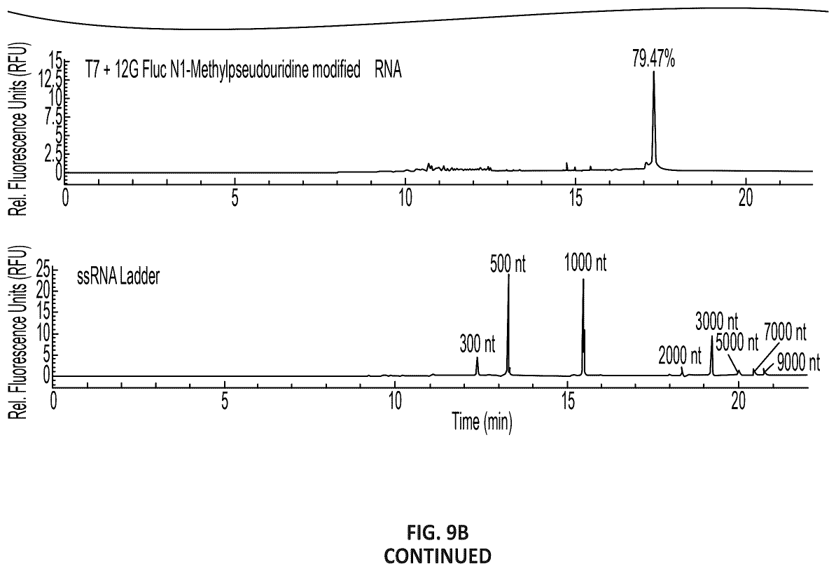

Similar to unmodified RNA, N1-methylpseudouridine modified RNA synthesized with DNA templates having poly-thymine or poly-guanine tracts at the 3′ end of the T7 promoter sequence maintained or decreased total RNA yield. By adding three, six, or twelve thymines at the 3′ end of the T7 promoter in the DNA template, RNA yield changed by approximately 1.00-fold, 0.37-fold and 0.22-fold, respectively, compared to yield of RNA made with a DNA template having the wild type T7 promoter (FIG. 8A). Moreover, adding three, six, or twelve extra guanines at the 3′end of the T7 promoter, resulted in a reduction of approximately 0.51-fold, 0.27-fold and 0.08-fold, respectively, in RNA yield compared to yield of RNA made with a DNA template having the wild type T7 promoter (FIG. 9A).

An electropherogram of the different sample traces detected by CE of poly-uridine bearing RNAs (made from DNA templates having poly-thymine tracts at the 3′ end of the T7 promoter) showed a similar profile to poly-adenine and poly-cytosine bearing RNAs (purity >85%) (FIG. 8B). In contrast to poly-adenine, poly-cytosine, and poly-uridine containing RNAs, only addition of 3- and 6-mer poly-guanine at the 3′ end of the T7 promoter in the DNA template generated modified full-length RNAs as the main product as seen in reference to the ssRNA ladder. Addition of a 12-mer poly-guanine tract at the 3′ end of the T7 promoter also generated several truncated RNA products and significantly decreased the purity of the RNA (<80%) (FIG. 9B).

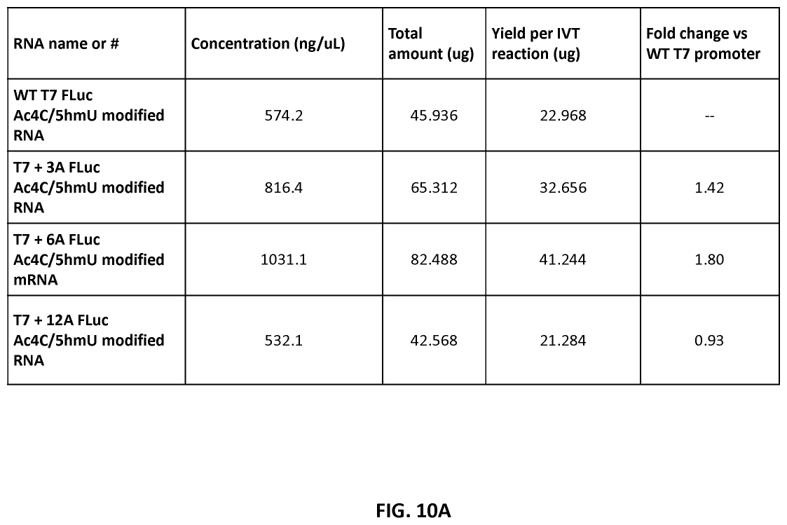

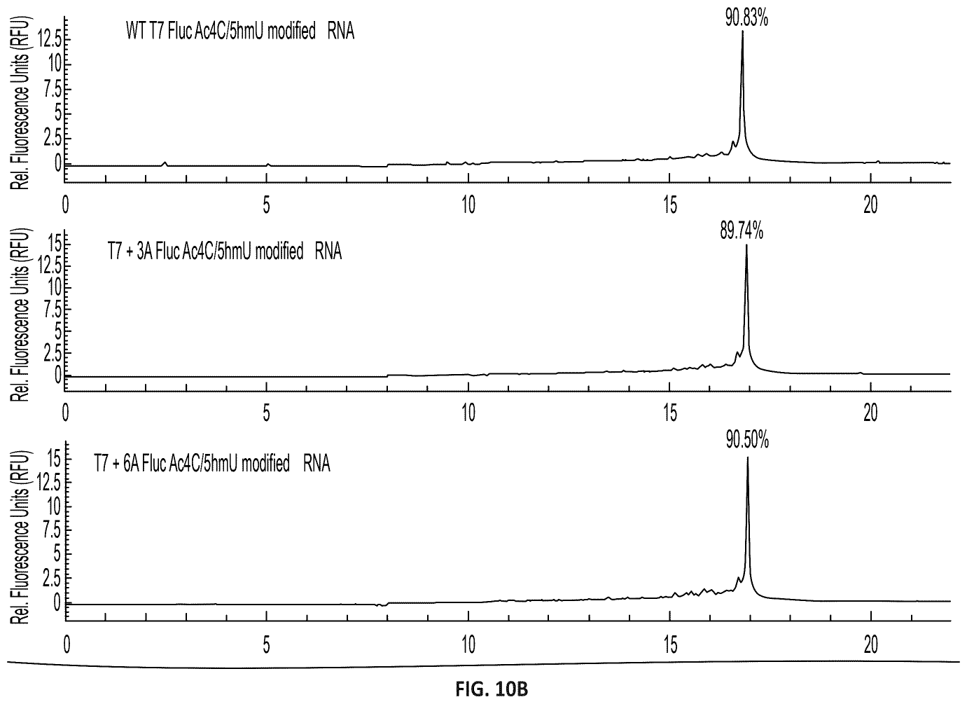

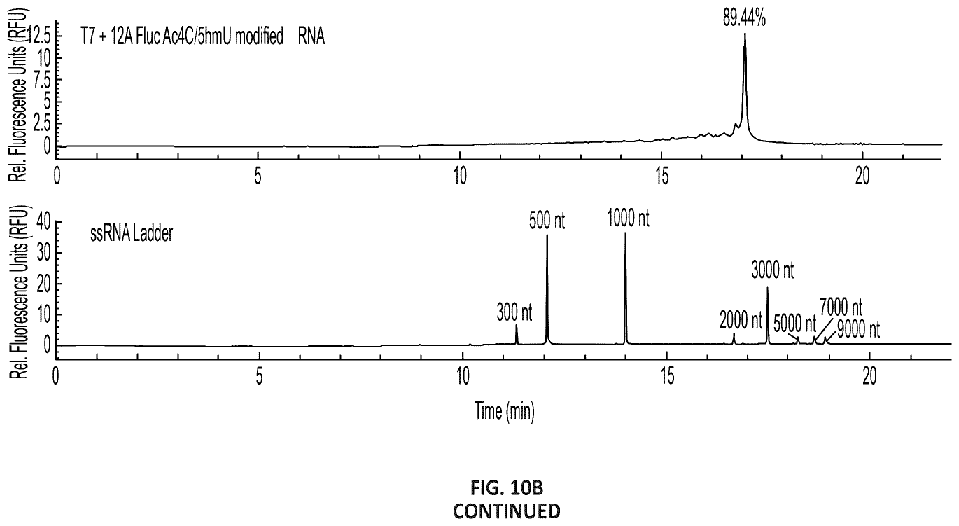

Using Ac4C/5hmU double modified RNA, only two of the DNA templates with increasing poly-adenine tracts generated a significant increase on RNA yield. By adding three, six, or twelve adenines at the 3′ end of the T7 promoter, RNA yield changed by approximately 1.42-fold, 1.80-fold and only 0.93-fold, respectively, compared to yield of RNA made with DNA templates having the wild type T7 promoter (FIG. 10A). The purity of the resulting RNA was >89% (FIG. 10B). As shown in FIG. 10B, the CE electropherogram of Ac4C/5hmU double modified RNA showed a different profile (with additional peaks at earlier timepoints) compared to the unmodified and N1-methylpseudouridine modified RNAs. Without wishing to be bound by any particular theory, this could represent non-denatured RNA molecules since modified cytosine nucleotides (e.g., Ac4C) can cause a stronger base pairing within RNA. See, e.g., Arango, D., et. al., 2018. Further without wishing to be bound by any particular theory, distinct traces can also represent different net charges emerging from both chemically modified nucleotides as opposed to the unmodified or N1-methylpseudouridine modified RNAs.

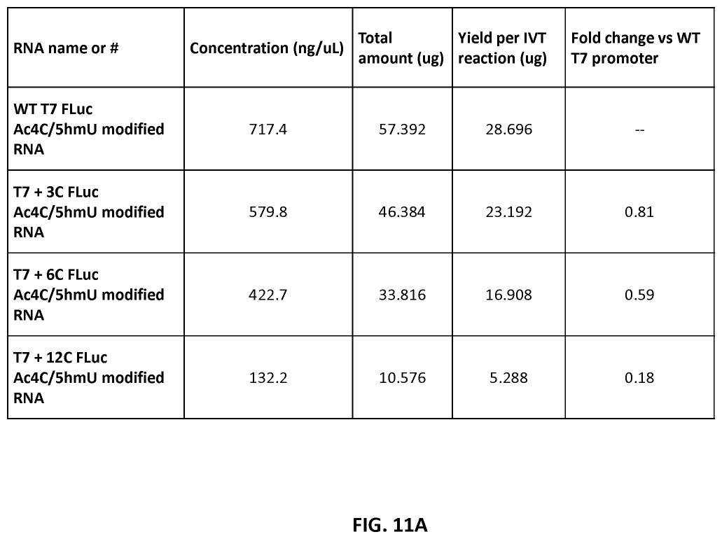

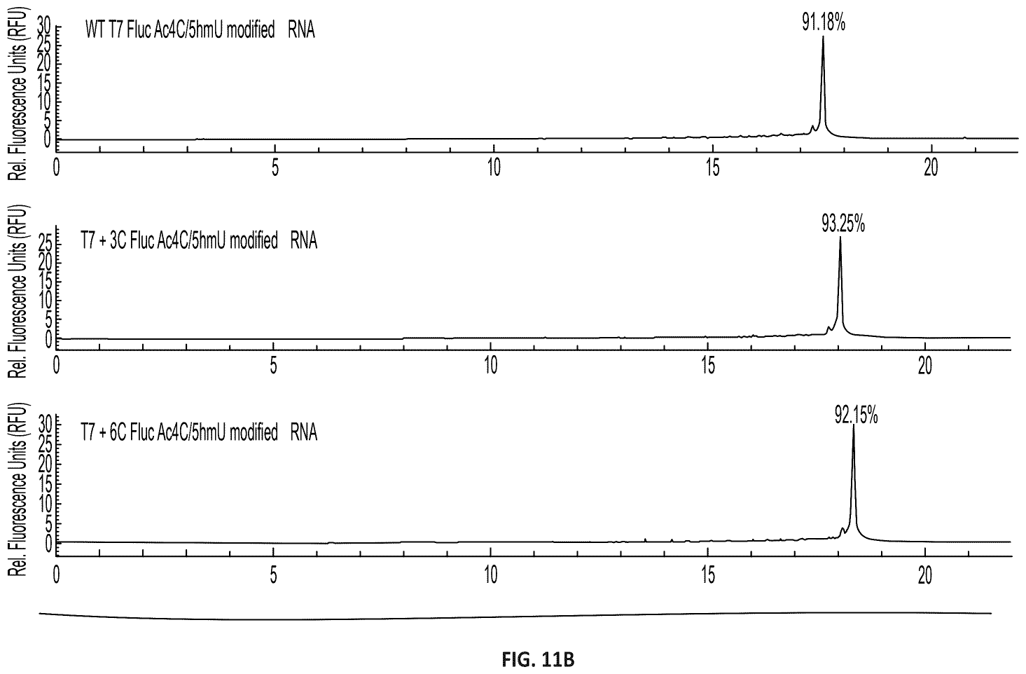

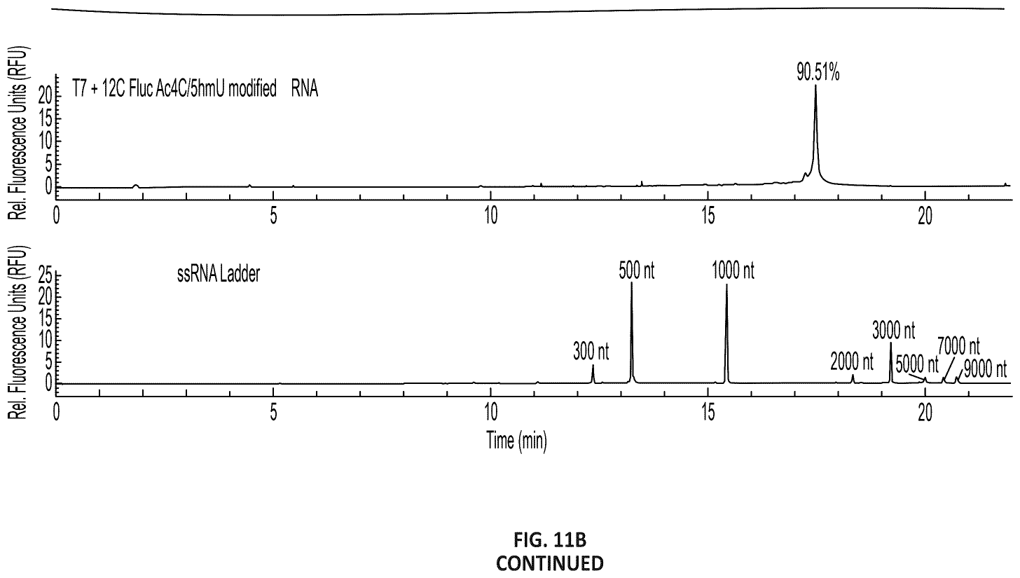

However, the effect on double modified RNA synthesized from DNA templates with increasing poly-cytosine tracts at the 3′ end of the T7 promoter sequence had a negative impact on RNA yield. By adding three, six, or twelve cytosines at the 3′ end of the T7 promoter, RNA yield reduced by 0.81-fold, 0.59-fold and only 0.18-fold, respectively, compared to the yield of double modified RNA made with DNA templates having a wild type T7 promoter (FIG. 11A). The purity of the resulting RNAs was >90% with a similar trace profile as observed with double modified RNA synthesized with DNA templates having a wild type T7 promoter (FIG. 11B).

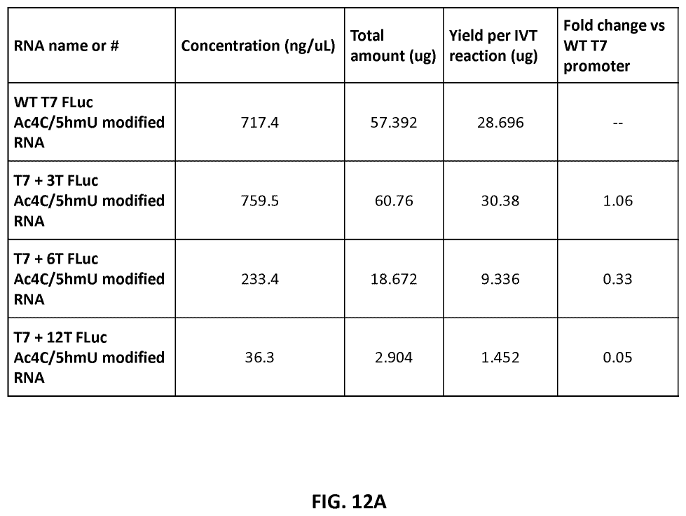

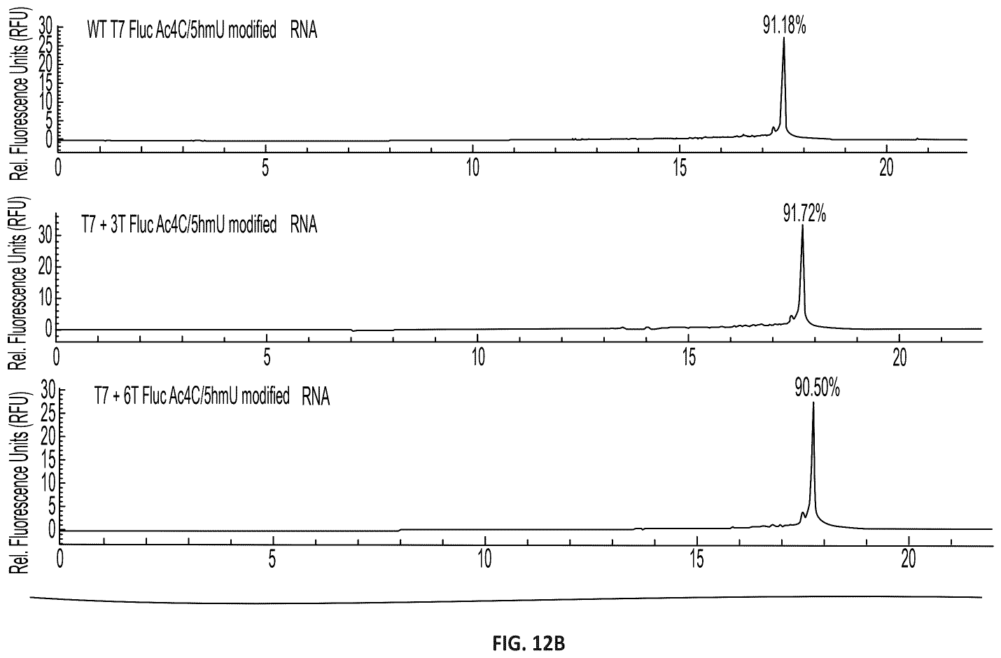

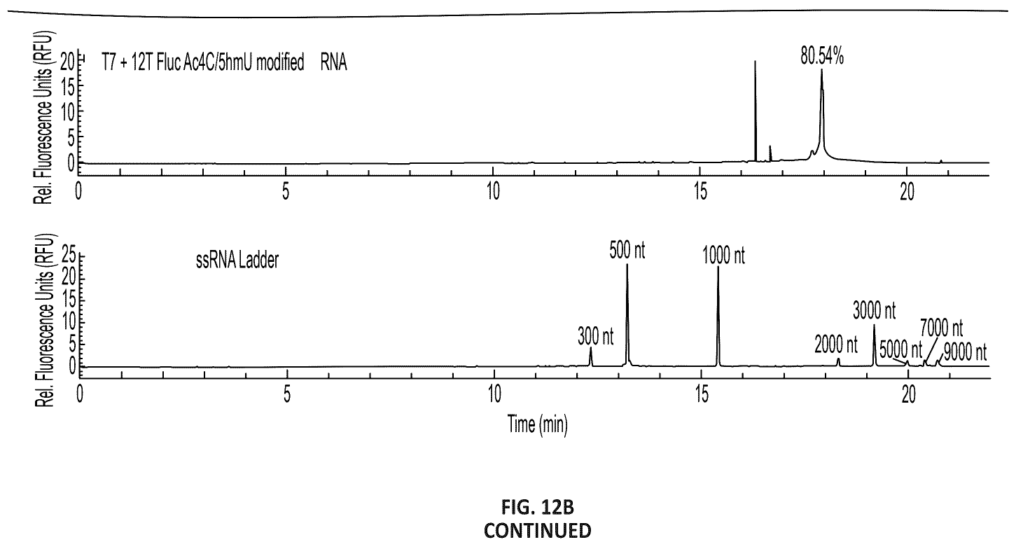

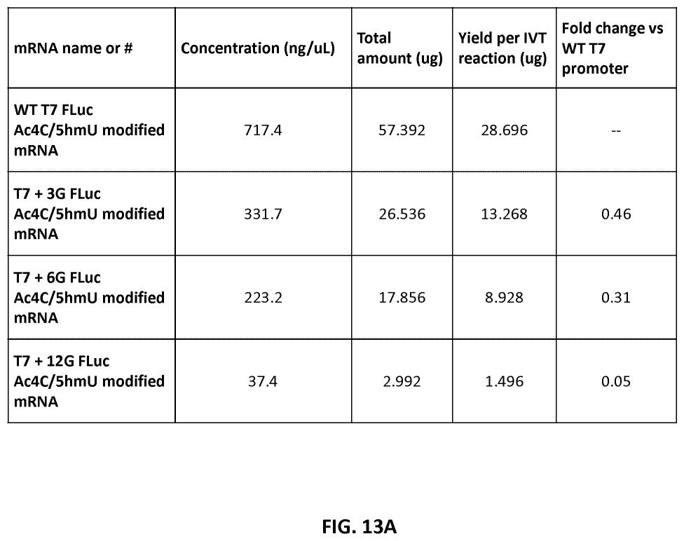

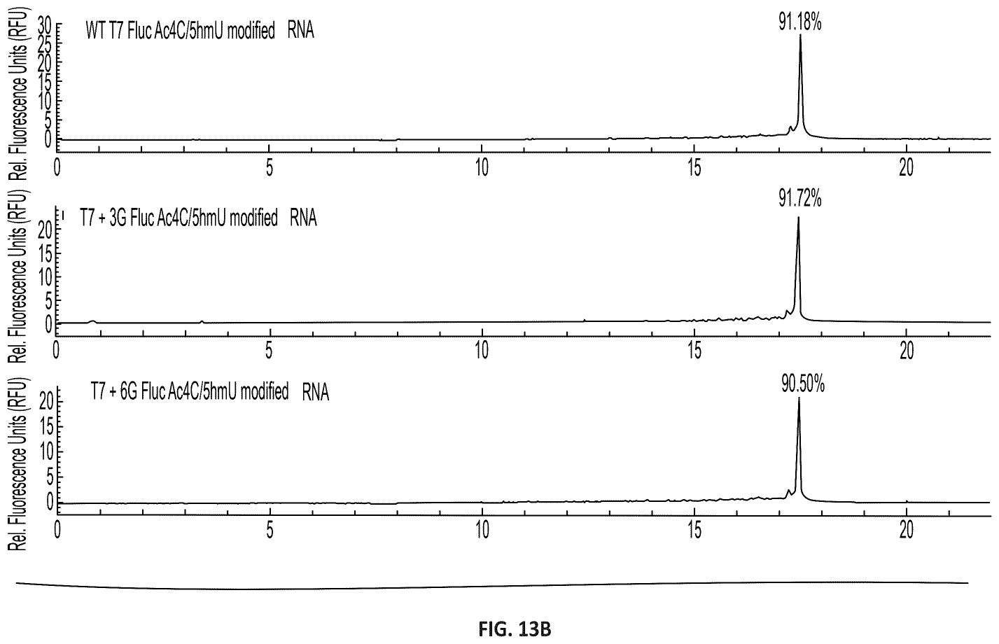

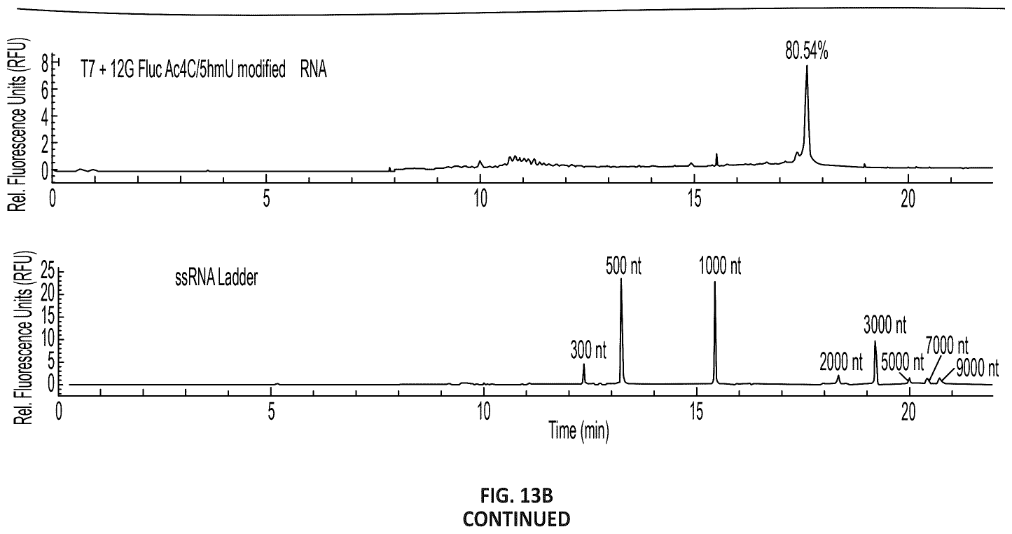

Similar to unmodified or N1-methylpseudouridine modified RNAs, addition of poly-thymine and poly-guanine tracks at the 3′ end of the T7 promoter decreased total double modified (ac4C/5hmU) RNA yield. By adding three, six, or twelve thymines to the 3′end of the T7 promoter in the DNA template, RNA yield changed by approximately 1.06-fold, 0.33-fold and 0.05-fold, respectively, compared to yield of RNA made with DNA template having the wild type T7 promoter (FIG. 12A). Moreover, as a result of adding three, six or twelve extra guanines, RNA yield changed by approximately 0.46-fold, 0.31-fold and 0.05-fold, respectively, compared yield of RNA made with DNA template having the wild type T7 promoter (FIG. 13A).

As shown in FIG. 12B, different sample traces were detected of 3- and 6-mer poly-uridine bearing RNAs (made with DNA templates having 3- or 6-mer thymines at the 3′end of the T7 promoter). RNA corresponding to the added 12-mer poly-uridine showed two distinct traces. Without wishing to be bound by theory, these traces may correspond to double stranded RNA (dsRNA), due to the earlier peak migration time and reduced total area with values of 9.16 and 1.40% in descendent migration time on the electropherogram, respectively. See for example, De Peña, A., “A microfluidic electrophoretic dual dynamic staining method for the identification and relative quantitation of dsRNA contaminants in Mrna vaccines,” Analyst, 2023, 148(16), 3758-3767, which is incorporated herein by reference in its entirety.

Similar to the other RNAs, only addition of 3- and 6-mer poly-guanine to the DNA template generated double modified (ac4C/5hmU) full-length RNAs as the main product. As expected, addition of a 12-mer poly-guanine generated several truncated RNA products, as shown by earlier migration times of the traces and decreased purity of the RNA (˜80%) (FIG. 13B).

Together, these results demonstrate that addition of poly-adenosine, poly-cytosine or poly-thymine tracts of varying lengths at the 3′ end of a T7 promoter in a polynucleotide improves the yield of RNAs made with such polynucleotides. The improved yield was observed with unmodified RNAs and RNAs comprising modified nucleotides. In addition, the increased yield of RNA was achieved while maintaining high RNA purity. Without wishing to be bound by any particular theory, in some embodiments, this observed increase in yield could be attributed to 1) increased flexibility of the region preceding the 5′-UTR, which in some embodiments can prevent stalling of the polymerase; and/or 2) allowance of the proper melting of the double stranded DNA in the initiation region.

The results provided herein also demonstrate that the beneficial effects on yield and/or purity observed in RNAs synthesized with polynucleotides having poly-adenosine, poly-cytosine and to a lesser extent poly-thymine tracts at the 3′ end of a promoter is not merely due to the length of the poly-adenosine, poly-cytosine or poly-thymine tracts added at the 3′ end of the promoter. This is evidenced by the data with poly-guanosine tracts at the 3′ end of the promoter which consistently reduced yield and/or purity of polyribonucleotides made with such polynucleotides. Without wishing to be bound by any particular theory, in some embodiments, the reduced yield and/or purity observed with poly-guanine tracts is due to structural properties related to having consecutive guanines that may interfere with RNA transcription and/or processing.

This data supports the utility of using polynucleotides comprising a promoter and a promoter proximal sequence (e.g., having poly-adenine, poly-cytosine and/or poly-thymine tracts) at the 3′ end of the promoter having two or more consecutive identical polynucleotides to make polyribonucleotides with high yield and/or purity. Such polyribonucleotides can be useful in downstream applications such as for use in therapeutic applications.

### Example 3: Use Of Polynucleotides Having a T7 Bacteriophage Promoter with Varying Lengths of Promoter Proximal Sequences For Generating RNA with 2′O Ribose

This Example describes use of exemplary DNA templates having poly-adenine tracts at the 3′ end of a T7 bacteriophage promoter for making RNA having 2′O ribose modifications.

DNA templates were generated encoding an exemplary payload (Fluc protein) with a wild type T7 bacteriophage promoter, or with a WT T7 promoter having poly-adenine tracts of increasing length at the 3′ end of the promoter: 3 adenines, 6 adenines, or 12 adenines. RNA was synthesized with 2′O ribose modified RNAs.

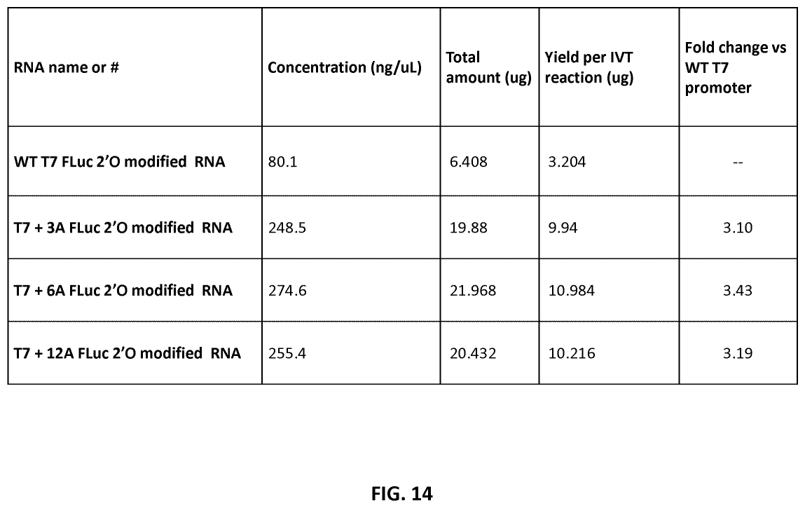

Addition of three, six or twelve adenines at the 3′ end of the T7 promoter in DNA templates used to generate 2′O ribose modified RNAs, increased RNA yield by approximately 3.10-fold, 3.43-fold and 3.19-fold, respectively, compared to the yield of RNA made using DNA template having the WT T7 promoter (FIG. 14).

These results demonstrate that polynucleotides having a poly-adenine tract at the 3′ end of a T7 promoter sequence can be used to generate 2′O ribose modified RNA with increased RNA yield and purity.

### Example 4: Use of Polynucleotides Having a 73 Bacteriophage Promoter with Varying Lengths of Promoter Proximal Sequences for Generating RNA

This Example describes use of exemplary DNA templates having poly-adenine tracts at the 3′ end of a T3 bacteriophage promoter for synthesizing RNA. DNA templates having increasing poly-adenine tracts at the 3′ end of the T3 promoter starting on the +4 position were made as described in Example 1.

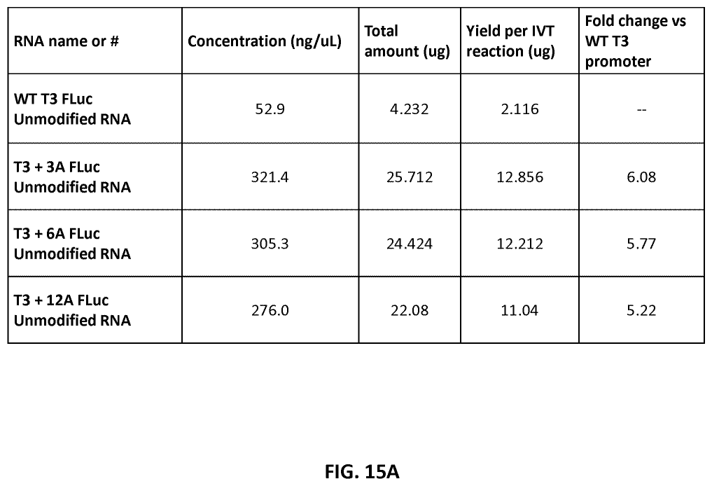

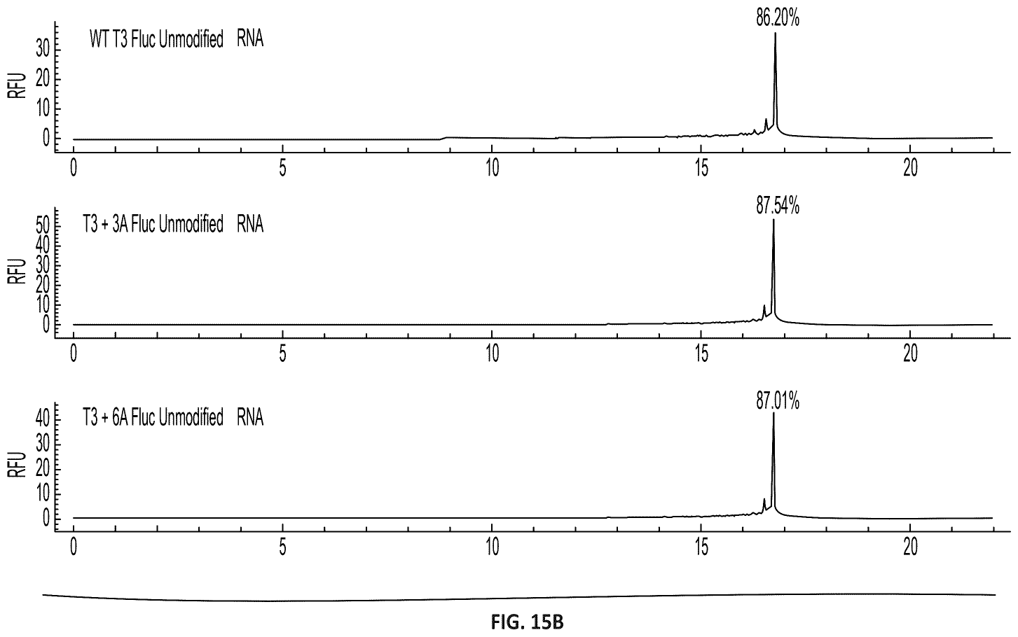

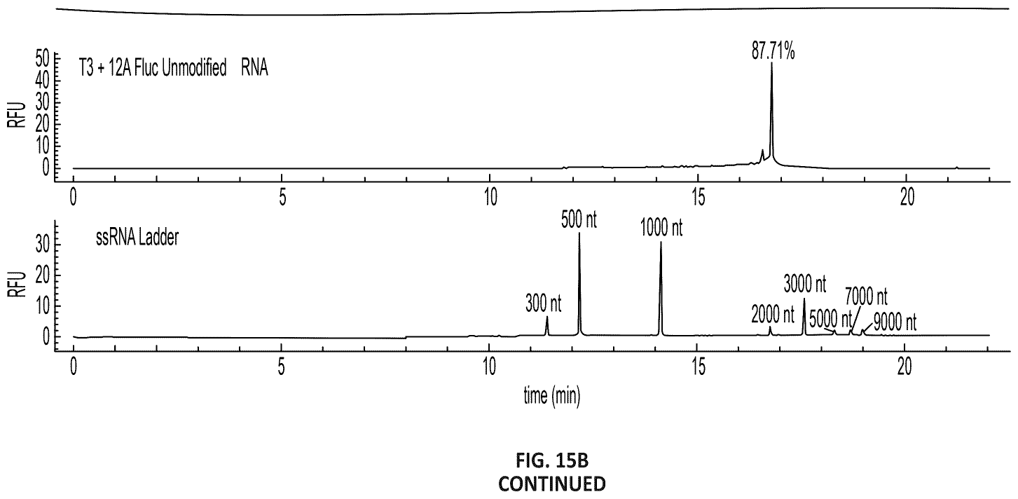

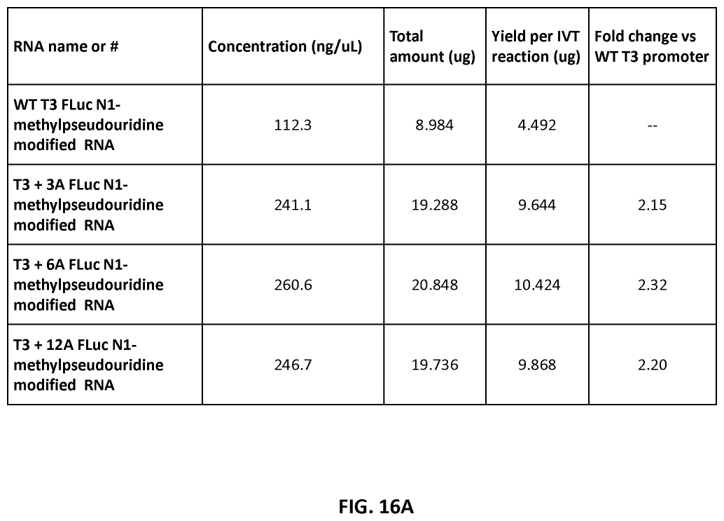

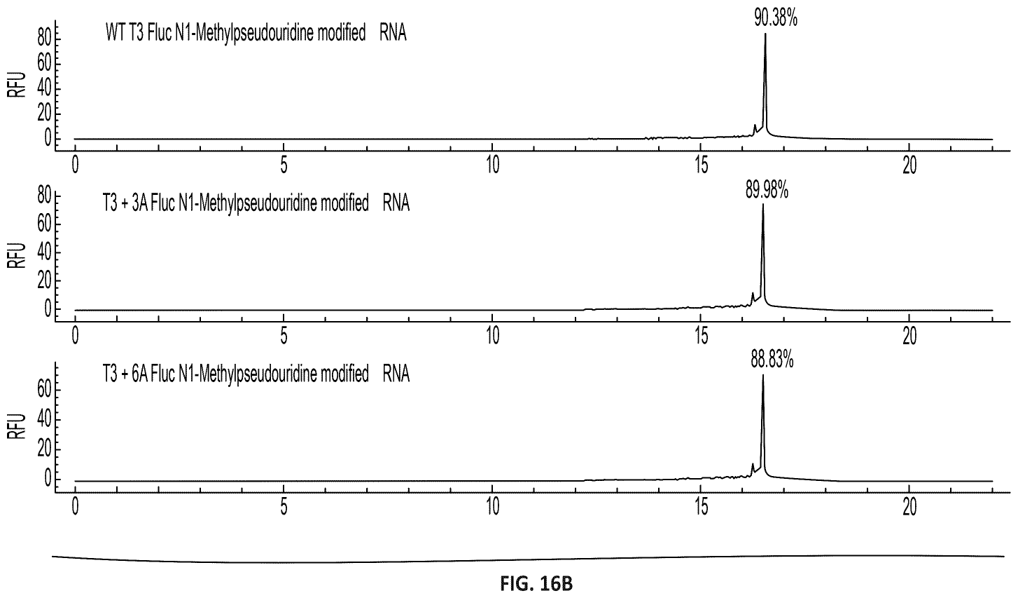

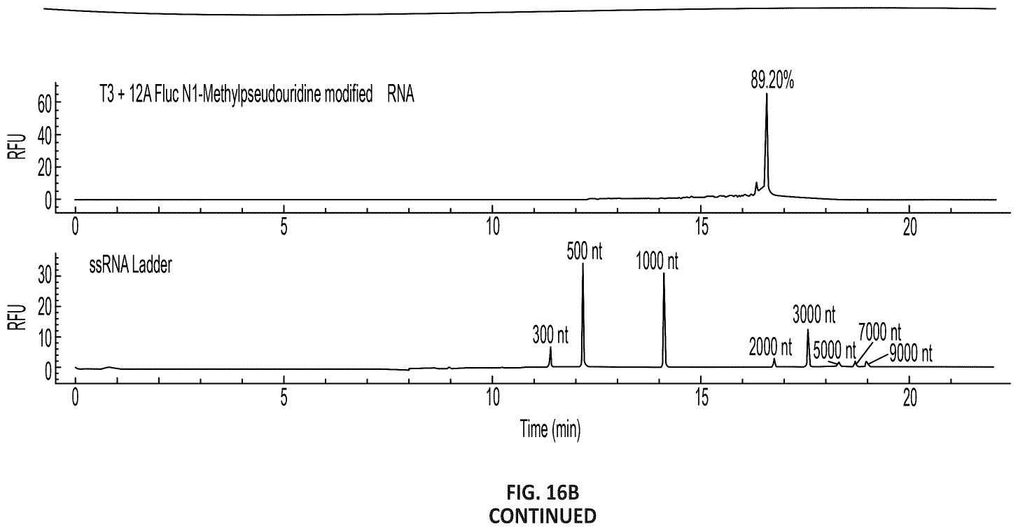

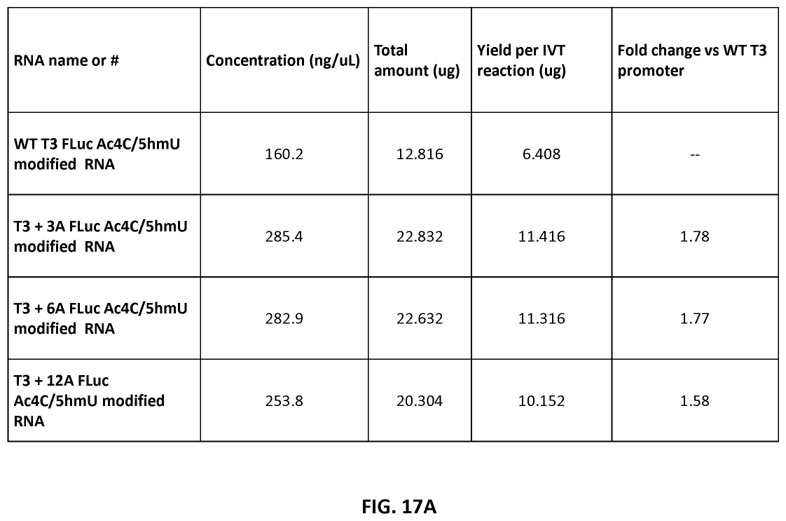

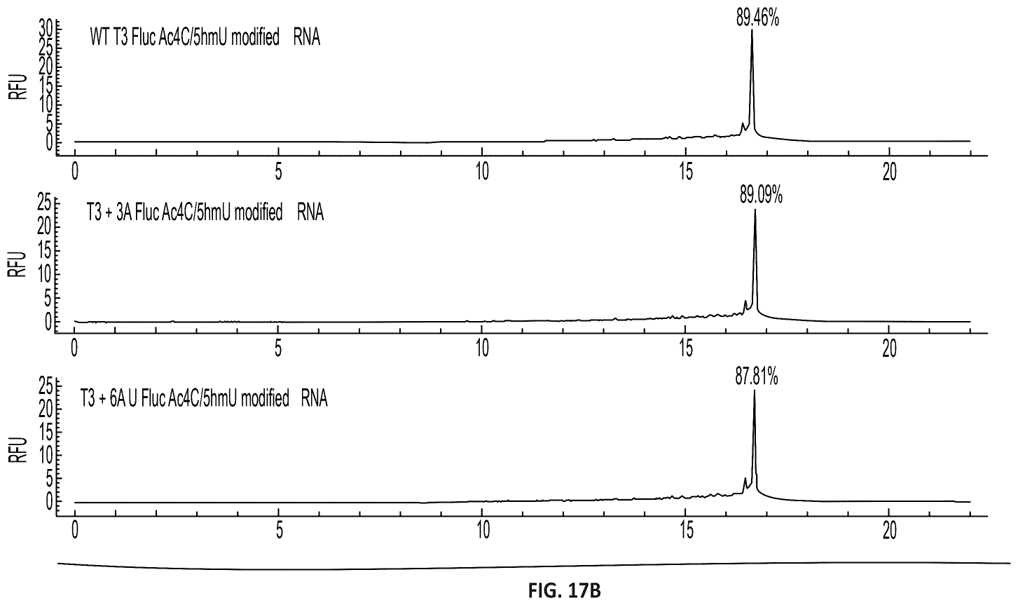

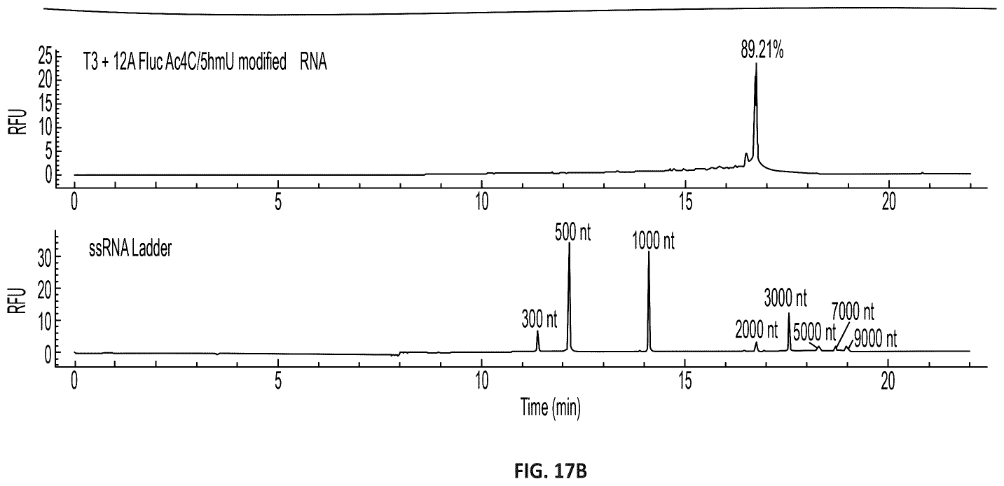

As shown in FIG. 15A, FIG. 16A, and FIG. 17A, an increase in total RNA yield was observed in unmodified, N1-methylpseudouridine modified, and Ac4C/5hmU double modified RNAs synthesized with DNA templates having a T3 promoter and increasing poly-A tracts at the 3′ end of the promoter. By adding three, six, or twelve adenines at the 3′ end of wild type T3 bacteriophage promoter, unmodified RNA yield increased by 6.08-fold, 5.77-fold and 5.22-fold, respectively, compared to yield of RNA made with a DNA template having the WT T3 promoter (FIG. 15A).

Similarly, by adding three, six or twelve extra adenines at the 3′ end of a wild type T3 promoter, N1-methylpseudouridine modified RNA yield increased approximately 2.15-fold, 2.32-fold and 2.20-fold, respectively (FIG. 16A). Increased yield of Ac4C/5hmU double modified RNA with addition of poly-adenine tracts at the 3′ end of the T3 promoter was also observed of 1.78-fold, 1.77-fold and 1.58-fold (FIG. 17A). Furthermore, FIG. 15B, FIG. 16B, and FIG. 17B illustrate electropherograms of the different sample traces detected by CE of exemplary RNAs synthesized with DNA templates having poly-adenine of varying lengths at the 3′ end of a wild type T3 promoter. According to CE analysis, all RNAs exhibited similar traces corresponding to full-length product and non-tailed impurities. Additionally, purity of RNAs bearing increasing poly-adenine tracts (made from DNA templates having poly-adenine tracts at the 3′ end of the promoter) similar to that observed in RNA made from DNA templates having the wildtype T3 promoter (purity >85%).

Together, these results demonstrate that addition of poly-adenine tracts of varying lengths at the 3′ end of a T3 promoter in a polynucleotide improves the yield of RNAs made with such polynucleotides The improved yield was observed with unmodified RNAs and RNAs comprising modified nucleotides. In addition, the increased yield of RNA was achieved while maintaining high RNA purity.

These results further demonstrate the unexpected finding that the activity of the T3 promoter (like the T7 promoter) can be manipulated by altering sequences at the 3′ end of the T3 promoter to improve the yield and/or purity of RNAs made with DNA templates having such a T3 promoter sequence.

This data supports the utility of using polynucleotides comprising a promoter and a promoter proximal sequence (e.g., having poly-adenine, poly-cytosine and/or poly-thymine tracts) at the 3′ end of the promoter having two or more consecutive identical polynucleotides to make polyribonucleotides with high yield and/or purity. Such polyribonucleotides can be useful in downstream applications such as for use in therapeutic applications.

### Example 5: Use Of Polynucleotides Having a SP6 Bacteriophage Promoter with Varying Lengths of Promoter Proximal Sequences for Generating RNA

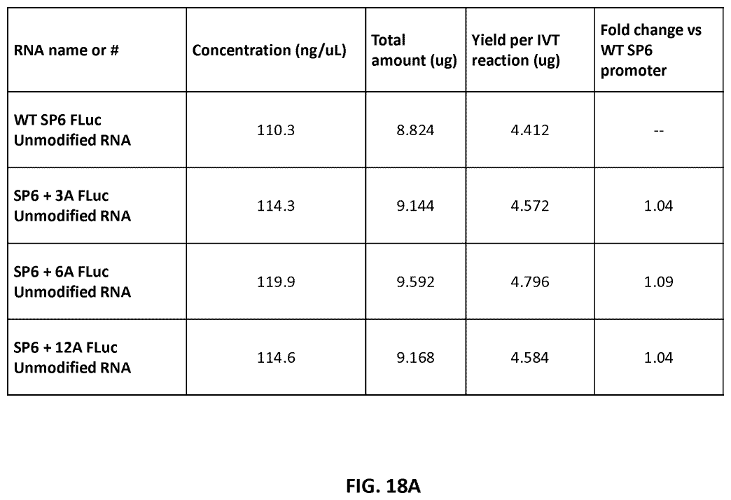

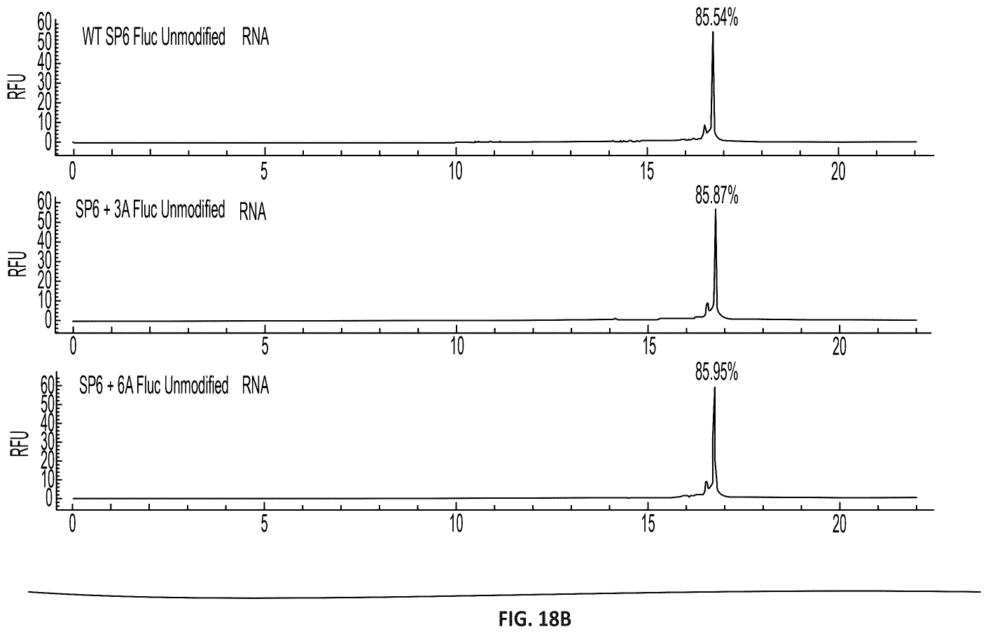

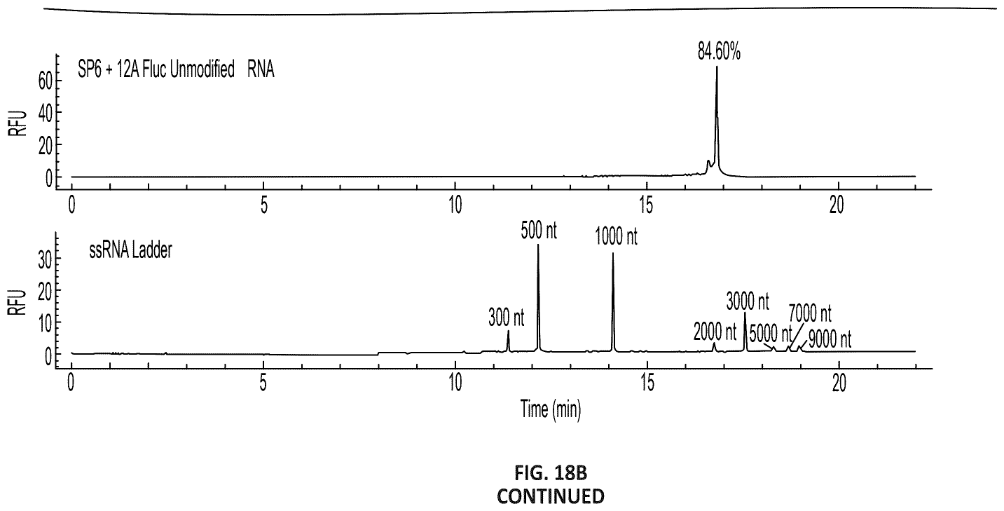

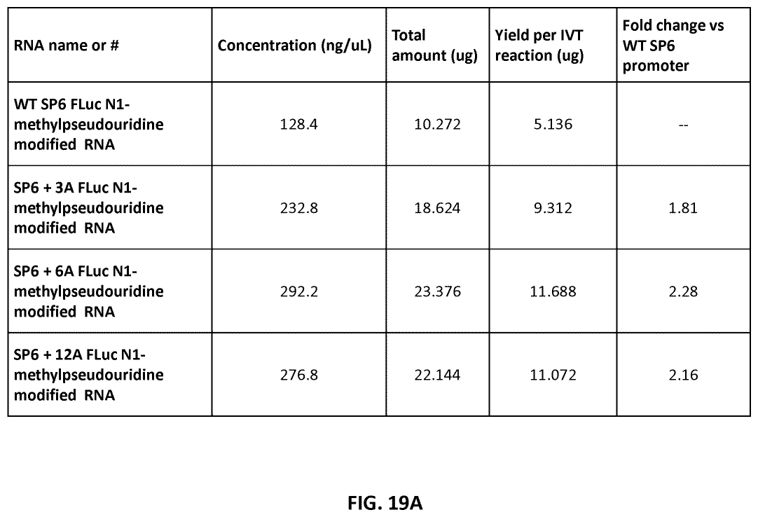

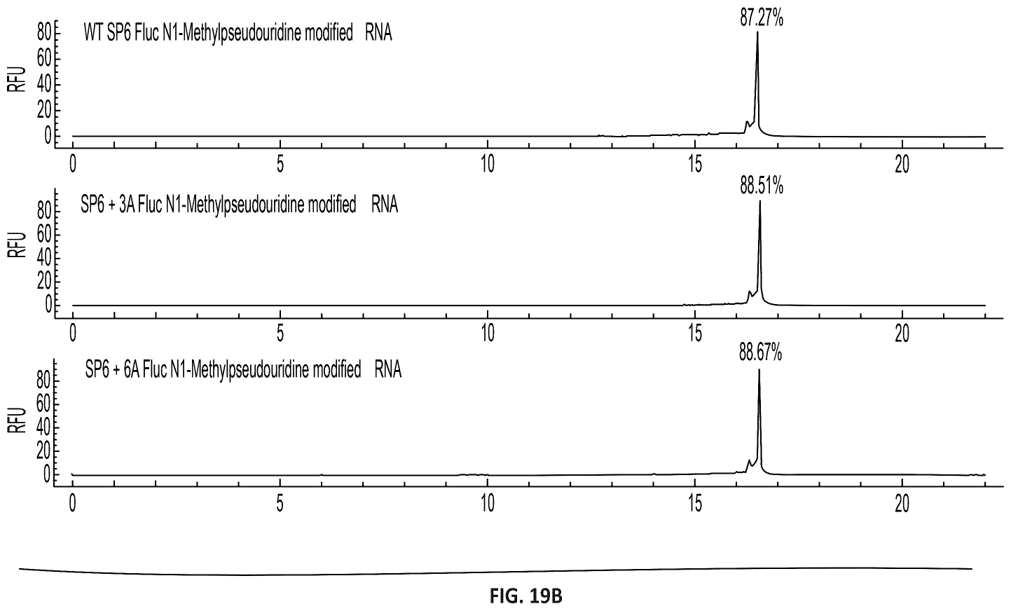

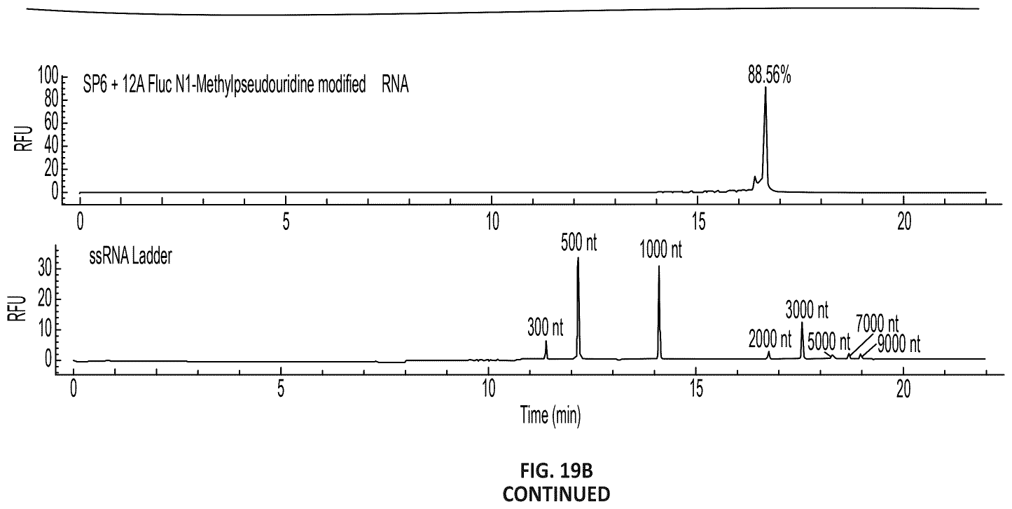

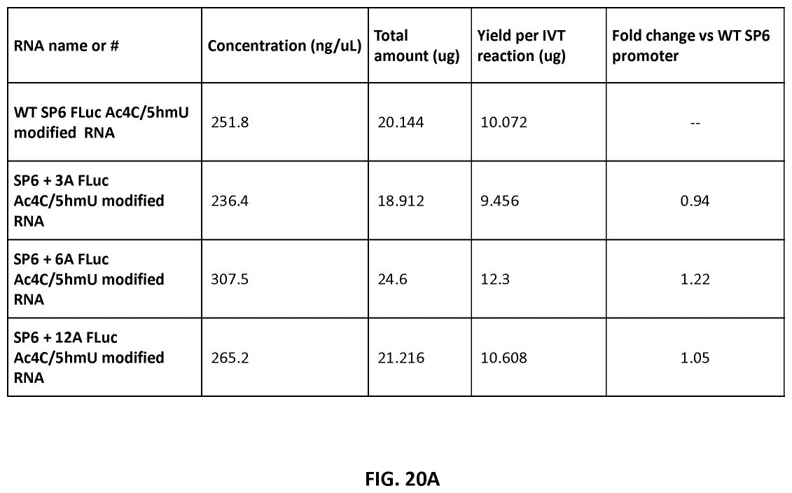

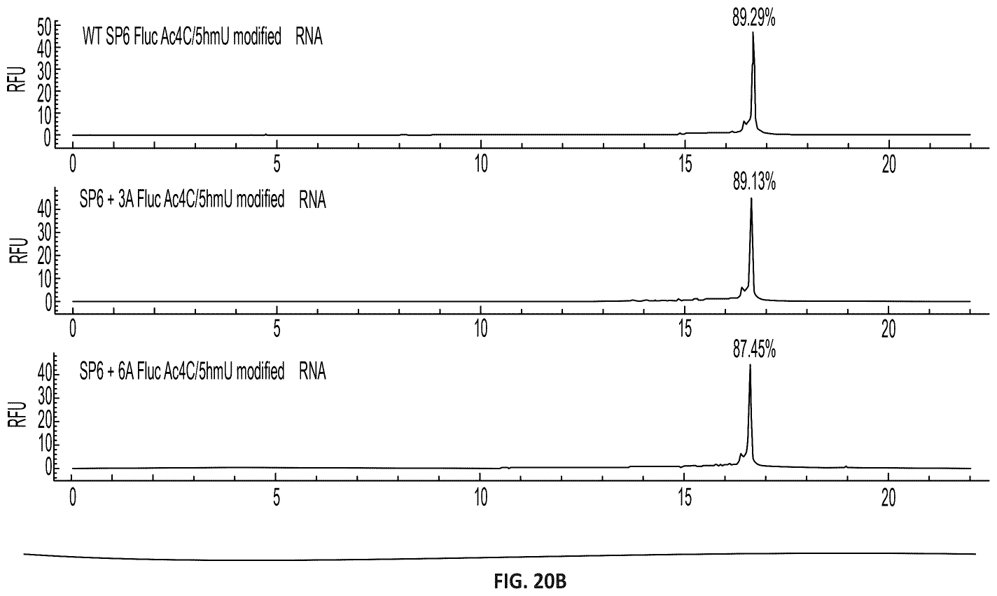

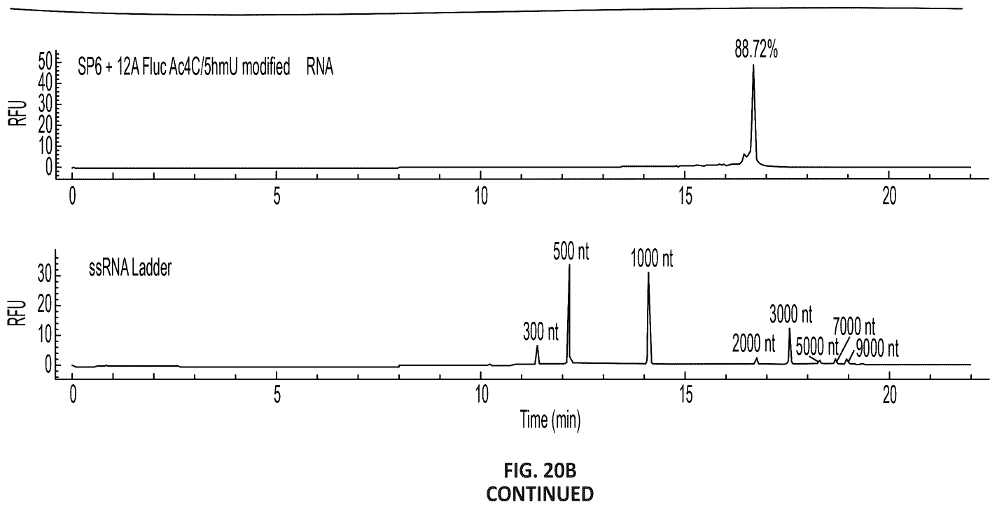

This Example describes use of exemplary DNA templates having poly-adenine tracts at the 3′ end of a SP6 bacteriophage promoter to synthesize RNA. As described herein, two different SP6 promoter variants were assessed: an AGG variant (SEQ ID NO: 27); and AGA variant (SEQ ID NO: 60). For the SP6 AGA variant, an increase in RNA yield was observed in RNA synthesized with N1-methylpseudouridine. The increase in yield was 1.81-fold with 3 adenines, 2.28-fold with 6 adenines, and 2.16-fold with 12 adenines, as compared to yield of RNA made with a DNA template having the SP6 AGA promoter without poly-adenine tracts at the 3′ end (FIG. 19A). The increase in RNA yield with unmodified nucleotides (FIG. 18A) or Ac4C/5hmU modifications (FIG. 20A), was more modest (˜1.04-fold to 1.09-fold and ˜0.94-fold to 1.22-fold increase in yield, respectively). FIG. 18B, FIG. 19B, and FIG. 20B illustrate a similar purity of RNAs (˜85% for the unmodified RNAs, >87% for the N1-methylpseudouridine and Ac4C/5hmU modified RNAs) synthesized with DNA templates having the SP6 AGA variant with increasing poly-adenine tracts at the 3′ end of the promoter as compared to RNA synthesized with a DNA template having the SP6 AGA promoter without poly-adenine tracts at the 3′ end of the SP6 AGA promoter.

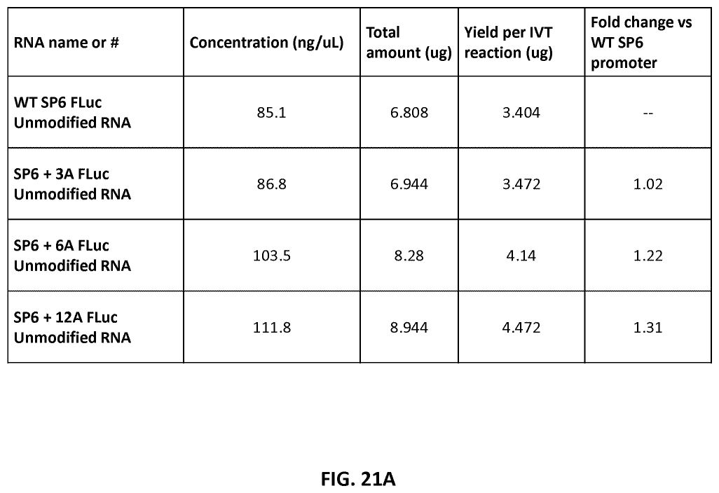

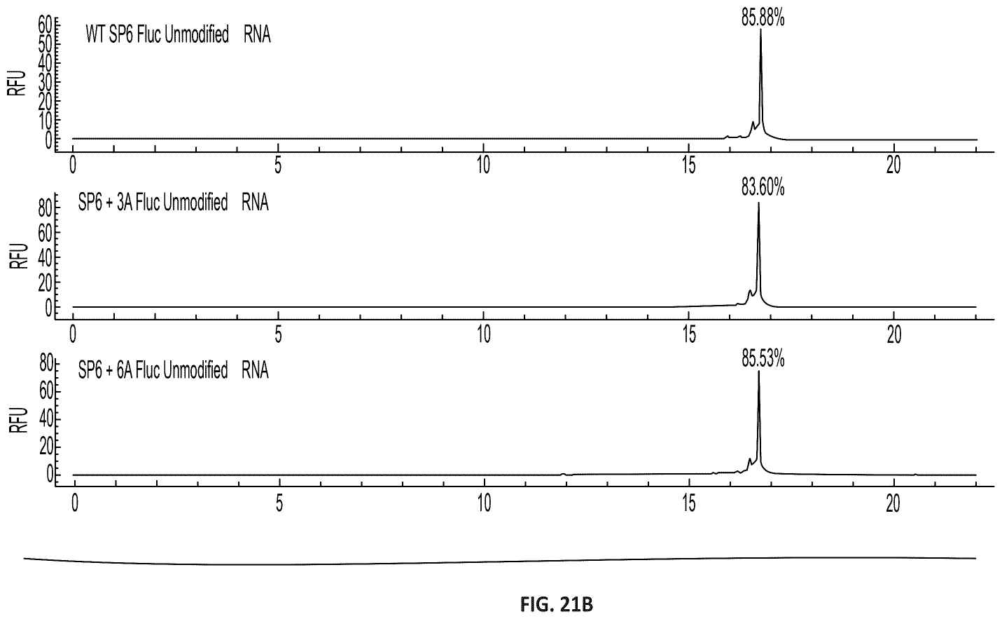

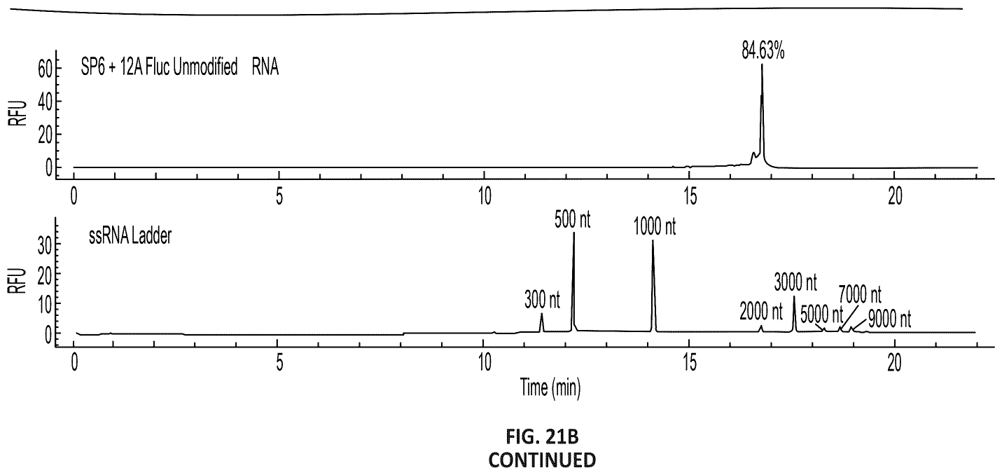

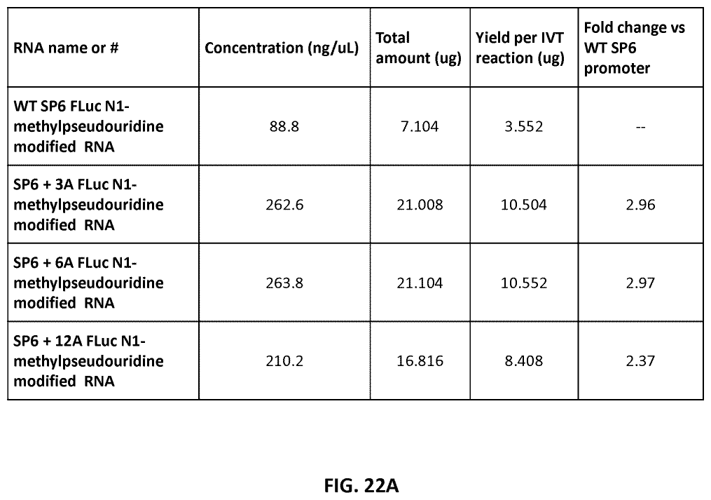

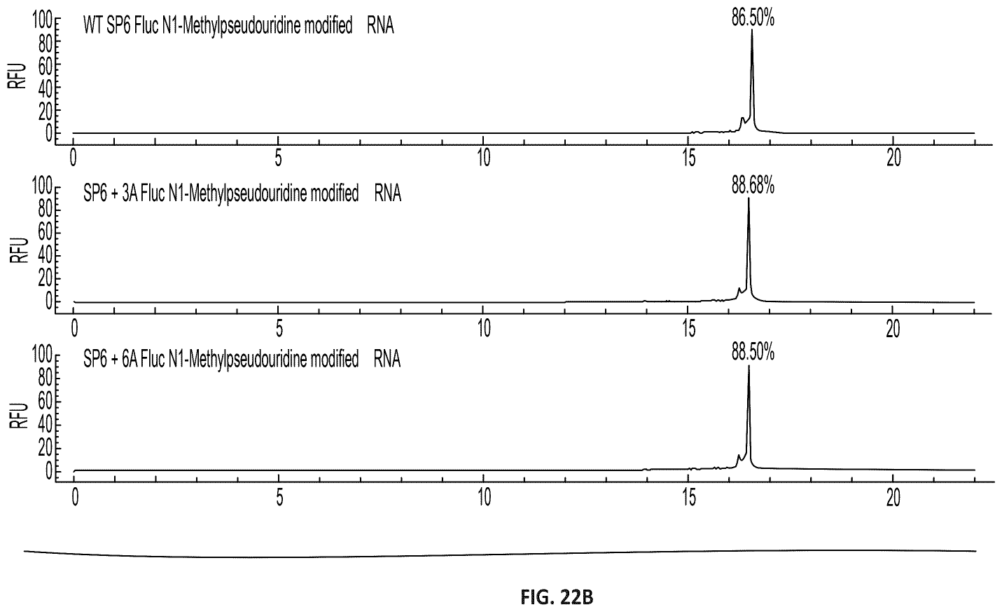

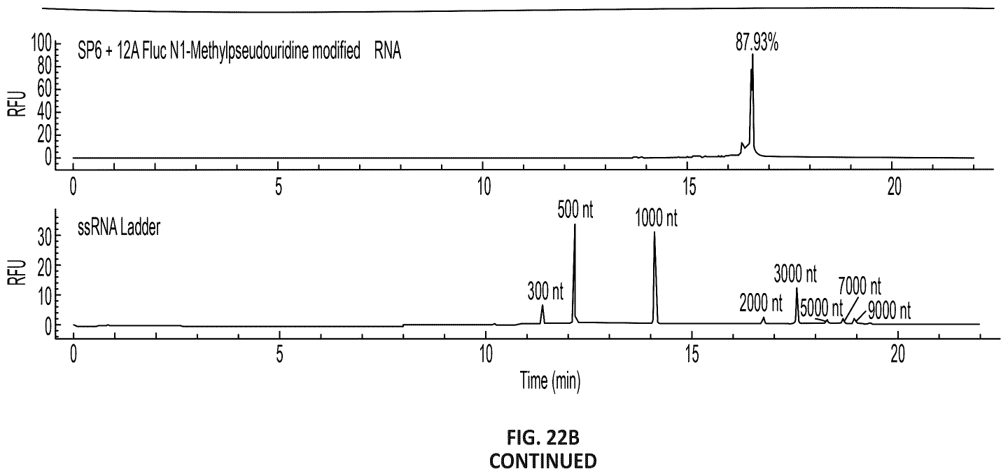

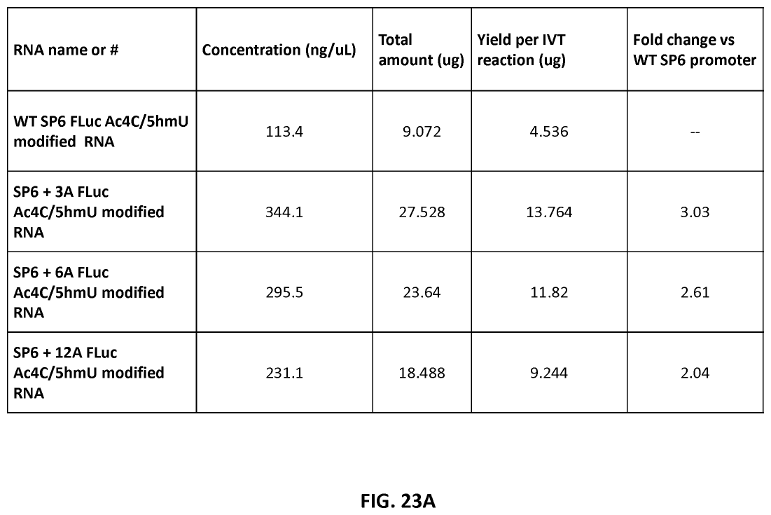

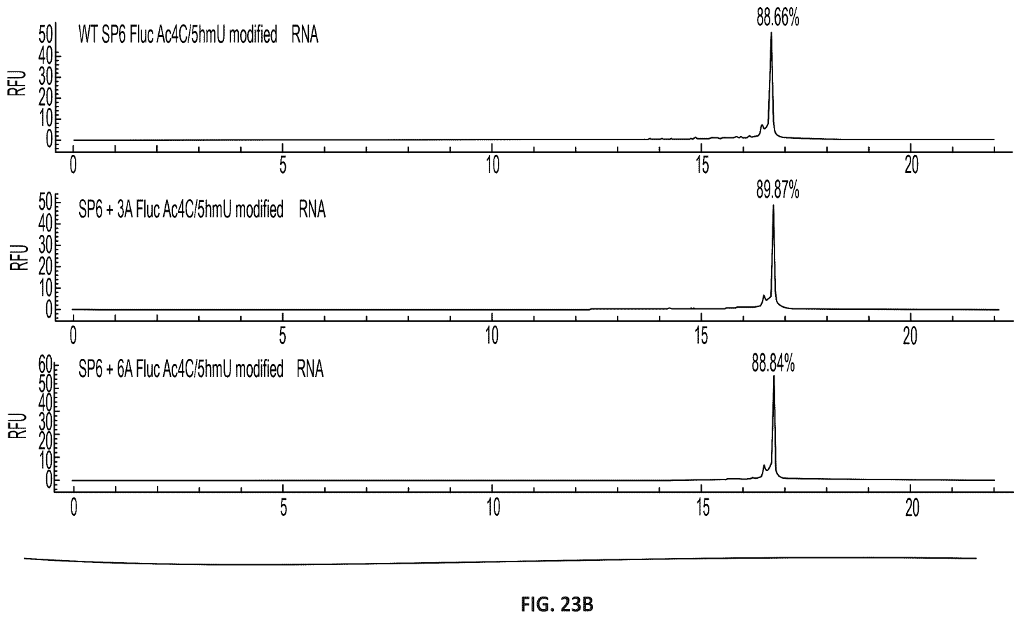

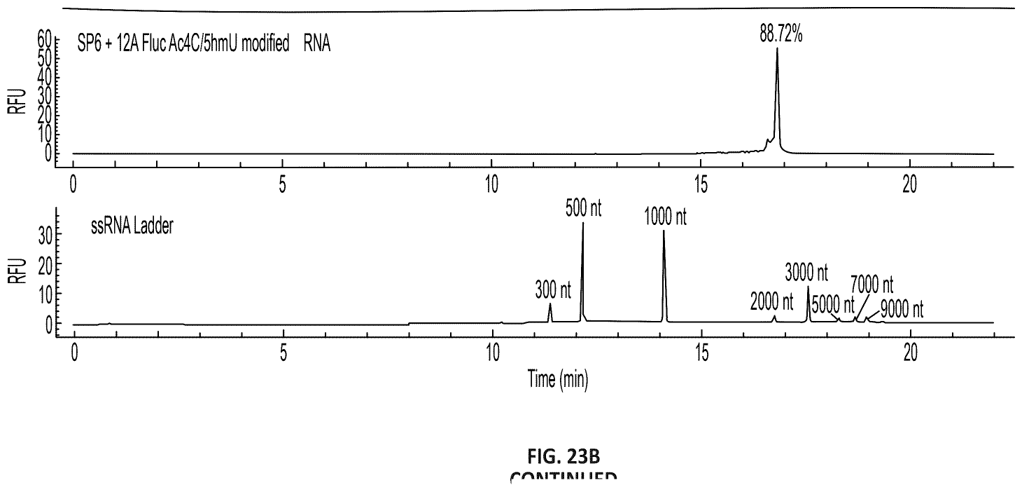

The SP6 AGG variant demonstrated an increased activity as compared to the SP6 AGA variant when poly-adenine tracts were added at the 3′ end of the promoter. Both N1-methylpseudouridine (FIG. 22A) and Ac4C/5hmU (FIG. 23A) modified RNAs made with DNA templates having increasing poly-adenine tracts at the 3′end of the SP6 AGG promoter showed an increase in RNA yield of approximately 3-fold as compared to yield of RNA made with DNA templates having the SP6 AGG promoter without poly-adenines at the 3′ end of the promoter. RNAs synthesized with unmodified nucleotides (FIG. 21A) exhibited only a modest increase in yield of about 1.02-fold to 1.31-fold.

Traces of RNAs generated with DNA templates having the SP6 AGG variant promoter and poly-adenine tracts at the 3′ end of the promoter (FIG. 21B, FIG. 22B, and FIG. 23B) was similar to RNA traced obtained with DNA templates having the SP6 AGG promoter control (˜85% for the unmodified RNAs, >86% for the N1-methylpseudouridine and Ac4C/5hmU modified RNAs).

Together, these results demonstrate that addition of poly-adenine tracts of varying lengths at the 3′ end of SP6 promoters in a polynucleotide improves the yield of RNAs made with such polynucleotides The improved yield was observed with unmodified RNAs and RNAs comprising modified nucleotides. In addition, the increased yield of RNA was achieved while maintaining high RNA purity.

These results further demonstrate the unexpected finding that the activity of a SP6 promoter (like the T7 promoter) can be manipulated by altering sequences at the 3′ end of a SP6 promoter to improve the yield and/or purity of RNAs made with DNA templates having such a SP6 promoter sequence.

This data supports the utility of using polynucleotides comprising a promoter and a promoter proximal sequence (e.g., having poly-adenine, poly-cytosine and/or poly-thymine tracts) at the 3′ end of the promoter having two or more consecutive identical polynucleotides to make polyribonucleotides with high yield and/or purity. Such polyribonucleotides can be useful in downstream applications such as for use in therapeutic applications.

### Example 6: Characterization of RNA Made with Polynucleotides Having a T7 Bacteriophage Promoter with Varying Lengths of Promoter Proximal Sequences

This Example describes cell viability and payload expression upon administration of RNAs synthesized with DNA templates having poly-adenine, poly-cytosine, poly-thymine, or poly-guanine tracts at the 3′ end of a T7 bacteriophage promoter.

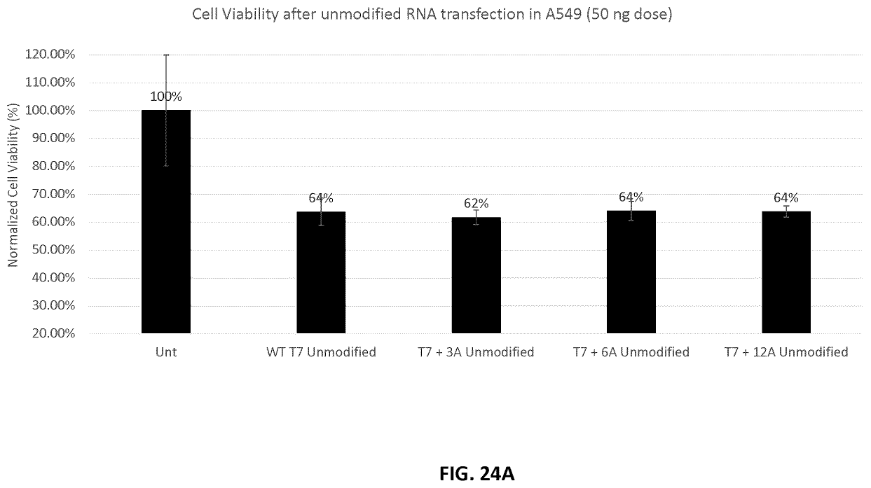

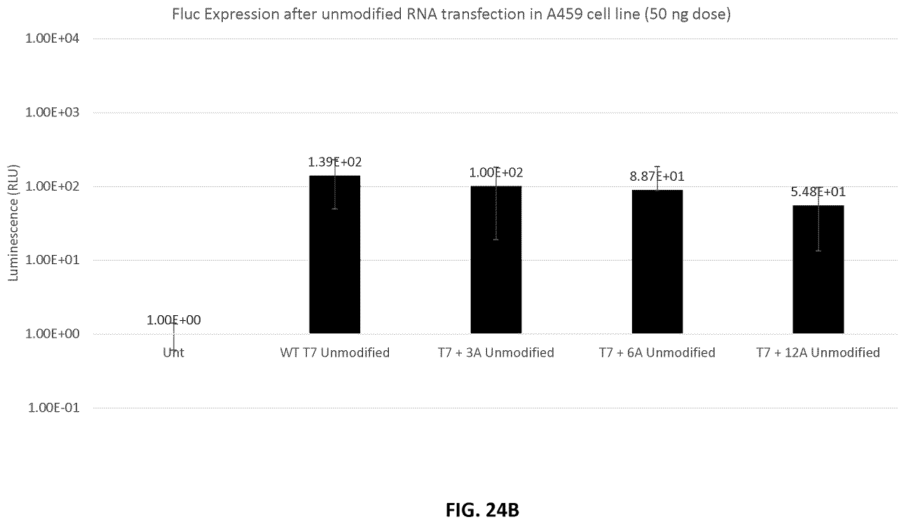

FIGS. 24A-24B show effects on viability and expression in the A549-Dual cell line of the different RNA samples made with in vitro transcription reactions utilizing exemplary DNA templates with increasing poly-adenine tracts at the 3′end of a T7 bacteriophage promoter. These results demonstrate that there was no significant difference in viability 72 hours post-transfection between experimental groups (61-64% on viability). There was a small decrease in luciferase expression as the poly-adenine tract increased in length (about 0.5-fold decrease in expression in the T7+12A group).

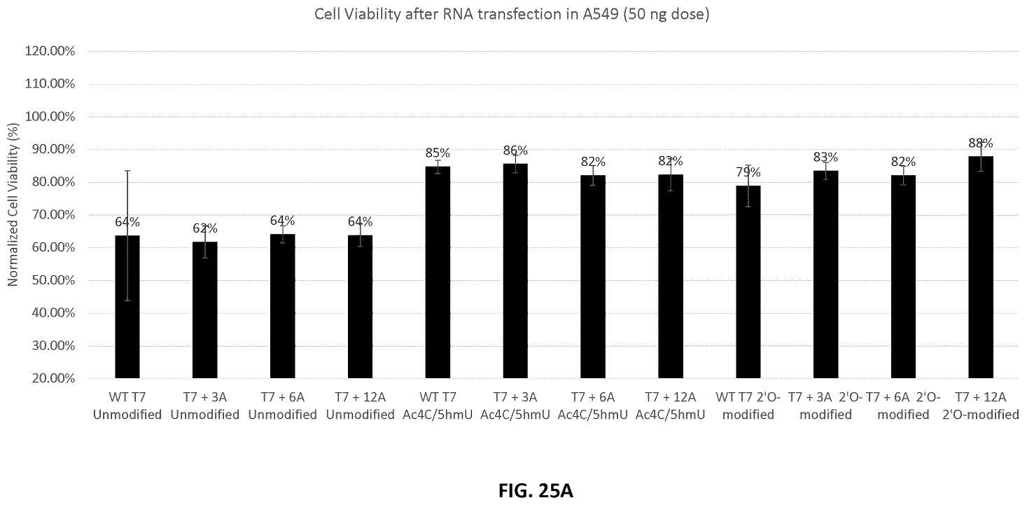

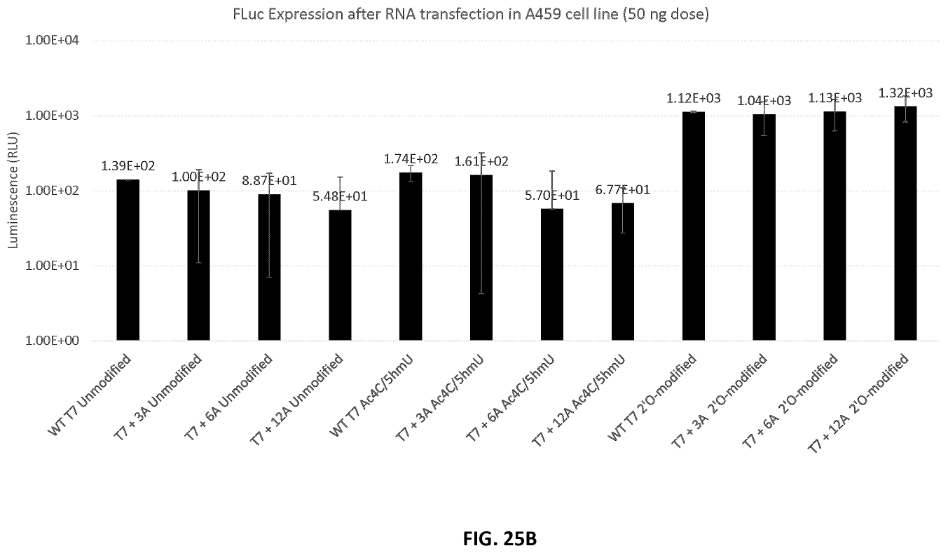

The payload expression and cell viability data obtained with poly-adenine tracts with unmodified RNAs (FIGS. 25A-25B) was similar to that obtained with poly-adenine tracts with modified RNAs (FIGS. 23A-23B) No significant differences in viability (FIG. 25A) was observed 72 hours post-transfection among the different unmodified and chemically modified RNAs. In general, there was a trend towards a decrease in luciferase expression (FIG. 25B) with increasing length of poly-adenine tracts in both unmodified and Ac4C/5hmU modified RNAs. RNAs having 2′O ribose modifications did not show a decrease in luciferase expression with increasing length of poly-adenine tracts.

The data provided herein showed an inverse correlation between the length of the poly-adenine tracts and luciferase expression on the same cell line with unmodified RNAs or RNAs modified with Ac4C/5hmU. Without wishing to be bound by any particular theory, in some embodiments, this decrease in expression with increased poly-adenine length could be attributed to transient binding of the Poly A binding protein (PABP) at the 5′ end of the transcript since it only needs a minimum of 5-mer adenosine residues for recognition. Additionally, without further wishing to be bound by any particular theory, the Lsm-complex that protects the 5′ end of polyadenylated RNAs from degradation (Bergman, et, al., 2007), can negatively influence the rate of translation.

Together, these results demonstrate that addition of poly-adenine tracts of different lengths at the 3′ end of the T7 promoter increased yield of an in vitro transcribed polyribonucleotide without significantly altering the cell viability or payload expression in cells administered the polyribonucleotides.

In summary, these results indicate that RNAs synthesized from polynucleotides having poly-adenine tracts at the 3′end of a promoter (e.g., T7) can be used in cellular applications (e.g., for administration to cells, tissue, or a subject) without impacting cell viability or payload expression.

### Example 7: Immunogenicity of RNA Made with Polynucleotides Having a 77 Bacteriophage Promoter with Varying Lengths of Promoter Proximal Sequences

This Example describes cell immunogenicity upon administration of RNAs synthesized with DNA templates having poly-adenine tracts at the 3′ end of a T7 bacteriophage promoter.

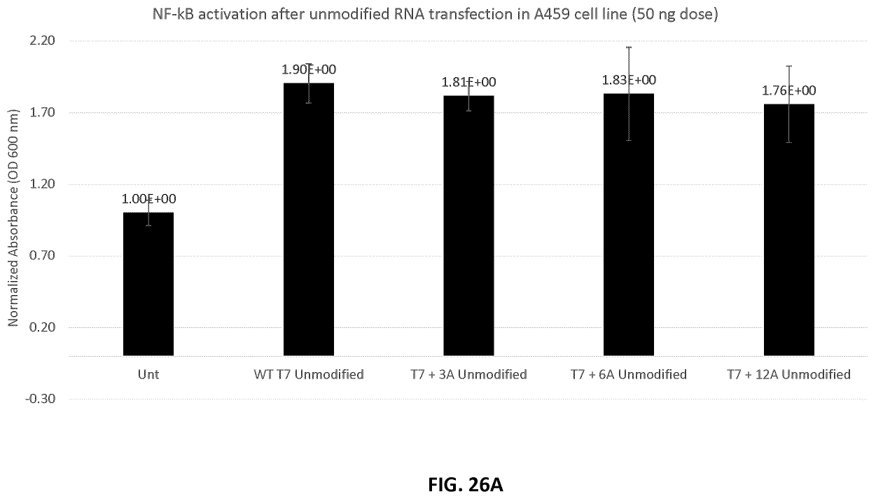

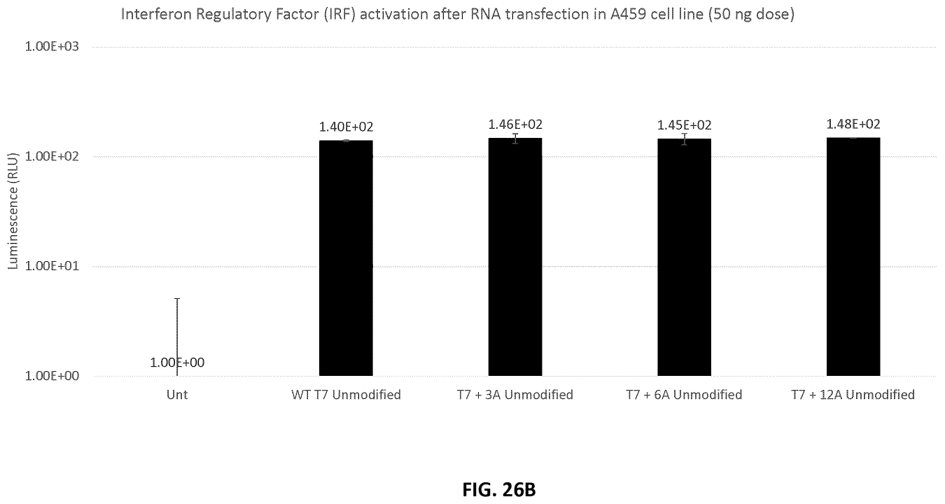

FIGS. 26A-26B demonstrates the effect on immunogenicity in A549-Dual cell line of the different RNA samples made with in vitro transcription reactions utilizing exemplary DNA templates with increasing poly-adenine tracts at the 3′end of a T7 bacteriophage promoter. NF-kB and IRF assays indicated no significant difference in immunogenicity between experimental groups.

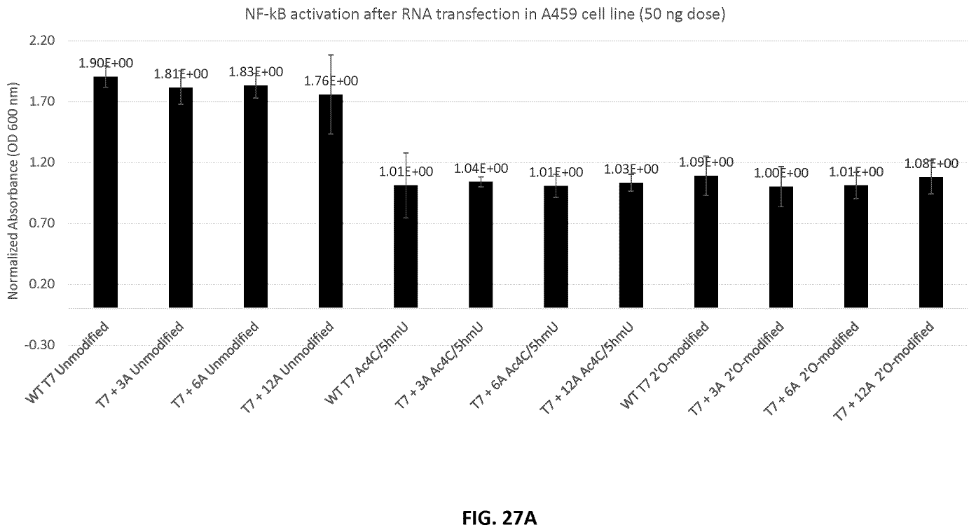

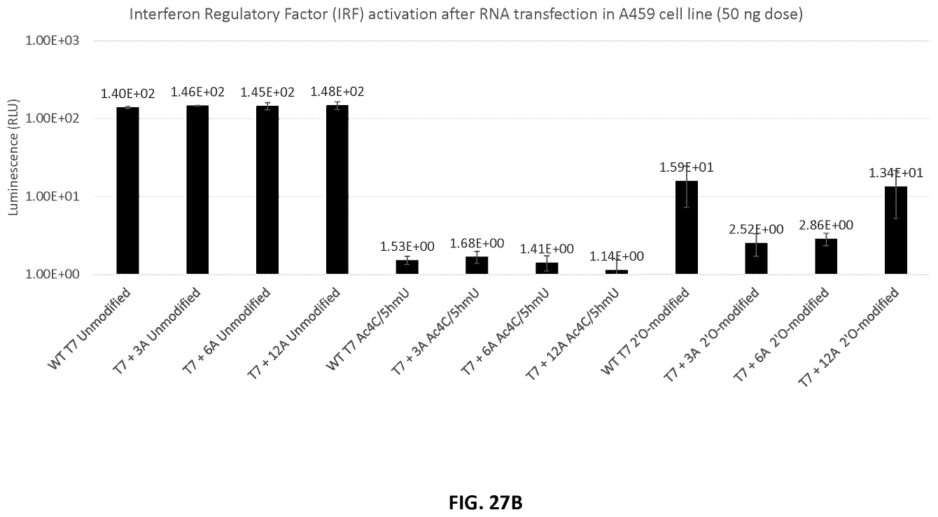

For Ac4C/5hmU modified RNAs a decrease in immunogenicity was observed even without the addition of poly-adenine tracts (i.e., with RNA synthesized with a DNA template having a T7 promoter, see fifth bar in FIG. 27A and FIG. 27B). This is in line with previous observations that Ac4C/5hmU modified RNAs reduce unwanted immunogenicity elicited by the RNA itself (as opposed to desired immunogenicity elicited by a payload encoded by the RNA). Additionally, Ac4C/5hmU modified RNAs having increasing tracts of poly-adenine (made with DNA templates having increasing tracts of poly-adenine at the 3′ end of the promoter sequence) further reduced IRF activation (FIG. 27B). A similar reduction in immunogenicity was observed with 2′-0 ribose modified RNAs having less than 12 poly-adenine tracts (FIGS. 27A-27B).

RNAs modified with N1-methylpseudouridine having poly-adenine tracts also had reduced immunogenicity in A549 cells as compared to immunogenicity observed with RNAs synthesized from a DNA template having a WT T7 promoter (data not shown). The highest immunogenicity signals were observed in RNAs having poly-guanine tracts that, as mentioned above in Example 3, generated numerous impurities likely corresponding to truncated RNA products which in some embodiments can trigger antiviral responses (Pichlmair, A., et. al., 2006; Rehwinkel, J., et. al., 2020; Wu, B., et. al., 2013).

Together, these results demonstrate that addition of poly-adenine tracts of different lengths at the 3′ end of the T7 promoter increased yield of an in vitro transcribed polyribonucleotide and reduced immunogenicity in cells administered the polyribonucleotide. In summary, these results indicate that RNAs synthesized from polynucleotides having poly-adenine tracts at the 3′ end of a promoter (e.g., T7) are beneficial for use in cellular applications (e.g., for administration to cells, tissue or a subject) due to the reduction in non-specific immunogenicity from the RNA itself (as opposed to desired immunogenicity elicited by a payload encoded by the RNA).

### EQUIVALENTS

Those skilled in the art will recognize, or be able to ascertain using no more than routine experimentation, many equivalents to the specific embodiments of the invention described herein. It is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Further, it should also be understood that any embodiment or aspect of the invention can be explicitly excluded from the claims, regardless of whether the specific exclusion is recited in the specification. The scope of the present invention is not intended to be limited to the above Description, but rather is as set forth in the claims that follow.

1.-52. (canceled)

53. A recombinant polynucleotide sequence, comprising:
(i) a promoter sequence comprising a 5′ end and a 3′ end;
(ii) a promoter proximal sequence adjacent to the 3′ end of the promoter sequence, wherein the promoter proximal sequence comprises four or more consecutive nucleotides having an adenine nucleobase; and
(iii) a sequence encoding a target operably linked to a 3′ end of (ii).

54. A recombinant polynucleotide sequence, comprising:
(i) a promoter sequence comprising a 5′ end and a 3′ end;
(ii) a promoter proximal sequence adjacent to the 3′ end of the promoter sequence, wherein the promoter proximal sequence comprises two or more consecutive nucleotides having a cytosine nucleobase; and
(iii) a sequence encoding a target operably linked to a 3′ end of (ii).

55. The recombinant polynucleotide of claim 53, wherein when paired with a complementary sequence of nucleotides, the promoter proximal sequence has a lower melting temperature than a comparable reference promoter proximal sequence.

56. The recombinant polynucleotide of claim 55, wherein the comparable reference promoter proximal sequence comprises:
(i) a lesser number of consecutive nucleotides comprising an adenine nucleobase or non-natural variants thereof as compared to the promoter proximal sequence; or
(ii) nucleotides having only guanine nucleobases or non-natural variants thereof.

57. The recombinant polynucleotide of claim 53, wherein the promoter proximal sequence is at least 5 nucleotides in length.

58. The recombinant polynucleotide of claim 53, wherein the promoter proximal sequence is at least 6 nucleotides in length.

59. The recombinant polynucleotide of claim 53, wherein the promoter proximal sequence is at least 9 nucleotides in length.

60. The recombinant polynucleotide of claim 53, wherein the promoter proximal sequence is at least 12 nucleotides in length.

61. The recombinant polynucleotide of claim 53, wherein the promoter is an RNA polymerase promoter or a fragment or a variant thereof.

62. The recombinant polynucleotide of claim 61, wherein the promoter is a bacteriophage promoter, a viral promoter, a bacterial promoter, a eukaryotic promoter or an engineered promoter, or a fragment or a variant of any of the foregoing.

63. The recombinant polynucleotide of claim 62, wherein the bacteriophage promoter is a T7 promoter or a variant or a fragment thereof, a T3 promoter or a variant or a fragment thereof, or an SP6 promoter or a variant or a fragment thereof.

64. The recombinant polynucleotide of claim 63, wherein the T7 promoter comprises:
(a) the sequence of SEQ ID NO: 6; or
(b) the sequence of SEQ ID NO: 70.

65. The recombinant polynucleotide of claim 63, wherein the T3 promoter comprises:
(a) the sequence of SEQ ID NO: 17; or
(b) the sequence of SEQ ID NO: 81.

66. The recombinant polynucleotide of claim 63, wherein the SP6 promoter comprises:
(a) the sequence of SEQ ID NO: 27; or
(b) the sequence of SEQ ID NO: 60.

67. The recombinant polynucleotide of claim 53, wherein the sequence encoding the target is situated 3′ of the promoter proximal sequence.

68. The recombinant polynucleotide of claim 53, wherein the target comprises a polyribonucleotide.

69. The recombinant polynucleotide of claim 53, further comprising one or more UTRs, a polyadenylation signal sequence, or a combination thereof.

70. The recombinant polynucleotide of claim 53, wherein the recombinant polyribonucleotide comprises one or more nucleosides comprising a modified nucleobase.

71. The recombinant polynucleotide of claim 70, wherein the modified nucleobase is an adenine, guanine, cytosine, or uracil.

72. The recombinant polynucleotide of claim 53, wherein the one or more nucleosides comprising a modified nucleobase comprises N4-acetyl-cytidine (ac4C), 5-hydroxymethyluridine (5-hmU), N1-methylpseudouridine, pyridin-4-one ribonucleoside, 5-aza-uridine, 6-aza-uridine, 2-thio-5-aza-uridine, 2-thio-uridine (s2U), 5-methyl cytidine (m5C), 5-aza-cytidine, 6-aza-cytidine, pseudoisocytidine, 3-methyl-cytidine (m3C), 5-formyl-cytidine (f5C), N4-methyl-cytidine (m4C), 2-amino-purine, 2, 6-diaminopurine, 2-amino-6-halo-purine, 6-halo-purine, inosine (I), 1-methyl-inosine (m1 I), wyosine (imG), methylwyosine (mimG), or any combination thereof.

73. The recombinant polynucleotide of claim 53, wherein the recombinant polyribonucleotide comprises one or more nucleosides comprising a modified ribose.

74. The recombinant polynucleotide of claim 73, wherein the modified ribose is 2′-O-acetylated.

75. A method of making a polyribonucleotide, comprising a step of incubating a transcription mixture comprising:
(i) a recombinant polynucleotide sequence comprising:
(a) a promoter sequence comprising a 5′ end and a 3′ end;
(b) a promoter proximal sequence adjacent to the 3′ end of the promoter sequence, wherein the promoter proximal sequence comprises two or more consecutive nucleotides, and
(c) a sequence encoding a target operably linked to a 3′ end of (b);
(ii) at least one RNA polymerase that recognizes the promoter sequence; and
(iii) a plurality of ribonucleotides comprising at least two different types of ribonucleotides, each type comprising a different nucleoside;
thereby producing the polyribonucleotide.

76. The method of claim 75, wherein the polyribonucleotide produced by the method is an in vitro transcribed polyribonucleotide.

77. The method of claim 75, wherein the transcription mixture further comprises a cap moiety to allow for co-transcriptional capping of the polyribonucleotide.

78. The method of claim 77, wherein the polyribonucleotide is produced at a higher yield compared to an otherwise similar polyribonucleotide made with a DNA template without a promoter proximal sequence or with a different promoter proximal sequence.

79. A polyribonucleotide made according to the method of claim 75.

80. A pharmaceutical composition comprising a polyribonucleotide of claim 53.

81. A method comprising delivering the pharmaceutical composition of claim 80.


SEQUENCE LISTING (93 sequences)

SEQ ID NO: 1 - DNA, 45 bp, synthetic construct
  source 1..45: mol_type = other DNA
gaatttaatacgactcactataaggcttgttctttttgcagaagc

SEQ ID NO: 2 - DNA, 48 bp, synthetic construct
  source 1..48: mol_type = other DNA
gaatttaatacgactcactataaggaaacttgttctttttgcagaagc

SEQ ID NO: 3 - DNA, 51 bp, synthetic construct
  source 1..51: mol_type = other DNA
gaatttaatacgactcactataaggaaaaaacttgttctttttgcagaagc

SEQ ID NO: 4 - DNA, 57 bp, synthetic construct
  source 1..57: mol_type = other DNA
gaatttaatacgactcactataaggaaaaaaaaaaaacttgttctttttgcagaagc

SEQ ID NO: 5 - DNA, 149 bp, synthetic construct
  source 1..149: mol_type = other DNA
ttttttttttttttttttttttttttttttttttttttttttttttttttttttttttttttttttttttttttttttttttttttttttttttttttttttttttttttttttttttttagaatgtgaagaaactttctttttattag

SEQ ID NO: 6 - DNA, 25 bp, synthetic construct
  source 1..25: mol_type = other DNA
gaatttaatacgactcactataagg

SEQ ID NO: 7 - DNA, 28 bp, synthetic construct
  source 1..28: mol_type = other DNA
gaatttaatacgactcactataaggaaa

SEQ ID NO: 8 - DNA, 31 bp, synthetic construct
  source 1..31: mol_type = other DNA
gaatttaatacgactcactataaggaaaaaa

SEQ ID NO: 9 - DNA, 37 bp, synthetic construct
  source 1..37: mol_type = other DNA
gaatttaatacgactcactataaggaaaaaaaaaaaa

SEQ ID NO: 10 - DNA, 40 bp, synthetic construct
  source 1..40: mol_type = other DNA
gaatttaatacgactcactataaggaaaaaaaaaaaaaaa

SEQ ID NO: 11 - DNA, 28 bp, synthetic construct
  source 1..28: mol_type = other DNA
gaatttaatacgactcactataaggttt

SEQ ID NO: 12 - DNA, 31 bp, synthetic construct
  source 1..31: mol_type = other DNA
gaatttaatacgactcactataaggtttttt

SEQ ID NO: 13 - DNA, 37 bp, synthetic construct
  source 1..37: mol_type = other DNA
gaatttaatacgactcactataaggtttttttttttt

SEQ ID NO: 14 - DNA, 28 bp, synthetic construct
  source 1..28: mol_type = other DNA
gaatttaatacgactcactataaggccc

SEQ ID NO: 15 - DNA, 31 bp, synthetic construct
  source 1..31: mol_type = other DNA
gaatttaatacgactcactataaggcccccc

SEQ ID NO: 16 - DNA, 37 bp, synthetic construct
  source 1..37: mol_type = other DNA
gaatttaatacgactcactataaggcccccccccccc

SEQ ID NO: 17 - DNA, 20 bp, synthetic construct
  source 1..20: mol_type = other DNA
aattaaccctcactaaaagg

SEQ ID NO: 18 - DNA, 23 bp, synthetic construct
  source 1..23: mol_type = other DNA
aattaaccctcactaaaaggaaa

SEQ ID NO: 19 - DNA, 26 bp, synthetic construct
  source 1..26: mol_type = other DNA
aattaaccctcactaaaaggaaaaaa

SEQ ID NO: 20 - DNA, 35 bp, synthetic construct
  source 1..35: mol_type = other DNA
aattaaccctcactaaaaggaaaaaaaaaaaaaaa

SEQ ID NO: 21 - DNA, 23 bp, synthetic construct
  source 1..23: mol_type = other DNA
aattaaccctcactaaaaggttt

SEQ ID NO: 22 - DNA, 26 bp, synthetic construct
  source 1..26: mol_type = other DNA
aattaaccctcactaaaaggtttttt

SEQ ID NO: 23 - DNA, 32 bp, synthetic construct
  source 1..32: mol_type = other DNA
aattaaccctcactaaaaggtttttttttttt

SEQ ID NO: 24 - DNA, 23 bp, synthetic construct
  source 1..23: mol_type = other DNA
aattaaccctcactaaaaggccc

SEQ ID NO: 25 - DNA, 26 bp, synthetic construct
  source 1..26: mol_type = other DNA
aattaaccctcactaaaaggcccccc

SEQ ID NO: 26 - DNA, 32 bp, synthetic construct
  source 1..32: mol_type = other DNA
aattaaccctcactaaaaggcccccccccccc

SEQ ID NO: 27 - DNA, 20 bp, synthetic construct
  source 1..20: mol_type = other DNA
atttaggtgacactataagg

SEQ ID NO: 28 - DNA, 23 bp, synthetic construct
  source 1..23: mol_type = other DNA
atttaggtgacactataaggaaa

SEQ ID NO: 29 - DNA, 26 bp, synthetic construct
  source 1..26: mol_type = other DNA
atttaggtgacactataaggaaaaaa

SEQ ID NO: 30 - DNA, 35 bp, synthetic construct
  source 1..35: mol_type = other DNA
atttaggtgacactataaggaaaaaaaaaaaaaaa

SEQ ID NO: 31 - DNA, 23 bp, synthetic construct
  source 1..23: mol_type = other DNA
atttaggtgacactataaggttt

SEQ ID NO: 32 - DNA, 26 bp, synthetic construct
  source 1..26: mol_type = other DNA
atttaggtgacactataaggtttttt

SEQ ID NO: 33 - DNA, 32 bp, synthetic construct
  source 1..32: mol_type = other DNA
atttaggtgacactataaggtttttttttttt

SEQ ID NO: 34 - DNA, 23 bp, synthetic construct
  source 1..23: mol_type = other DNA
atttaggtgacactataaggccc

SEQ ID NO: 35 - DNA, 26 bp, synthetic construct
  source 1..26: mol_type = other DNA
atttaggtgacactataaggcccccc

SEQ ID NO: 36 - DNA, 32 bp, synthetic construct
  source 1..32: mol_type = other DNA
atttaggtgacactataaggcccccccccccc

SEQ ID NO: 37 - DNA, 48 bp, synthetic construct
  source 1..48: mol_type = other DNA
gaatttaatacgactcactataaggtttcttgttctttttgcagaagc

SEQ ID NO: 38 - DNA, 51 bp, synthetic construct
  source 1..51: mol_type = other DNA
gaatttaatacgactcactataaggttttttcttgttctttttgcagaagc

SEQ ID NO: 39 - DNA, 57 bp, synthetic construct
  source 1..57: mol_type = other DNA
gaatttaatacgactcactataaggttttttttttttcttgttctttttgcagaagc

SEQ ID NO: 40 - DNA, 48 bp, synthetic construct
  source 1..48: mol_type = other DNA
gaatttaatacgactcactataaggccccttgttctttttgcagaagc

SEQ ID NO: 41 - DNA, 51 bp, synthetic construct
  source 1..51: mol_type = other DNA
gaatttaatacgactcactataaggcccccccttgttctttttgcagaagc

SEQ ID NO: 42 - DNA, 57 bp, synthetic construct
  source 1..57: mol_type = other DNA
gaatttaatacgactcactataaggcccccccccccccttgttctttttgcagaagc

SEQ ID NO: 43 - DNA, 48 bp, synthetic construct
  source 1..48: mol_type = other DNA
gaatttaatacgactcactataagggggcttgttctttttgcagaagc

SEQ ID NO: 44 - DNA, 51 bp, synthetic construct
  source 1..51: mol_type = other DNA
gaatttaatacgactcactataaggggggggcttgttctttttgcagaagc

SEQ ID NO: 45 - DNA, 57 bp, synthetic construct
  source 1..57: mol_type = other DNA
gaatttaatacgactcactataaggggggggggggggcttgttctttttgcagaagc

SEQ ID NO: 46 - DNA, 40 bp, synthetic construct
  source 1..40: mol_type = other DNA
aattaaccctcactaaaaggcttgttctttttgcagaagc

SEQ ID NO: 47 - DNA, 43 bp, synthetic construct
  source 1..43: mol_type = other DNA
aattaaccctcactaaaaggaaacttgttctttttgcagaagc

SEQ ID NO: 48 - DNA, 46 bp, synthetic construct
  source 1..46: mol_type = other DNA
aattaaccctcactaaaaggaaaaaacttgttctttttgcagaagc

SEQ ID NO: 49 - DNA, 52 bp, synthetic construct
  source 1..52: mol_type = other DNA
aattaaccctcactaaaaggaaaaaaaaaaaacttgttctttttgcagaagc

SEQ ID NO: 50 - DNA, 40 bp, synthetic construct
  source 1..40: mol_type = other DNA
atttaggtgacactataaggcttgttctttttgcagaagc

SEQ ID NO: 51 - DNA, 43 bp, synthetic construct
  source 1..43: mol_type = other DNA
atttaggtgacactataaggaaacttgttctttttgcagaagc

SEQ ID NO: 52 - skipped

SEQ ID NO: 53 - DNA, 46 bp, synthetic construct
  source 1..46: mol_type = other DNA
atttaggtgacactataaggaaaaaacttgttctttttgcagaagc

SEQ ID NO: 54 - DNA, 52 bp, synthetic construct
  source 1..52: mol_type = other DNA
atttaggtgacactataaggaaaaaaaaaaaacttgttctttttgcagaagc

SEQ ID NO: 55 - DNA, 40 bp, synthetic construct
  source 1..40: mol_type = other DNA
atttaggtgacactataagacttgttctttttgcagaagc

SEQ ID NO: 56 - DNA, 43 bp, synthetic construct
  source 1..43: mol_type = other DNA
atttaggtgacactataagaaaacttgttctttttgcagaagc

SEQ ID NO: 57 - DNA, 46 bp, synthetic construct
  source 1..46: mol_type = other DNA
atttaggtgacactataagaaaaaaacttgttctttttgcagaagc

SEQ ID NO: 58 - DNA, 52 bp, synthetic construct
  source 1..52: mol_type = other DNA
atttaggtgacactataagaaaaaaaaaaaaacttgttctttttgcagaagc

SEQ ID NO: 59 - DNA, 1840 bp, synthetic construct
  source 1..1840: mol_type = other DNA
cttgttctttttgcagaagctcagaataaacgctcaactttggccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaataatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 60 - DNA, 20 bp, synthetic construct
  source 1..20: mol_type = other DNA
atttaggtgacactataaga

SEQ ID NO: 61 - DNA, 23 bp, synthetic construct
  source 1..23: mol_type = other DNA
atttaggtgacactataagaaaa

SEQ ID NO: 62 - DNA, 26 bp, synthetic construct
  source 1..26: mol_type = other DNA
atttaggtgacactataagaaaaaaa

SEQ ID NO: 63 - DNA, 32 bp, synthetic construct
  source 1..32: mol_type = other DNA
atttaggtgacactataagaaaaaaaaaaaaa

SEQ ID NO: 64 - DNA, 23 bp, synthetic construct
  source 1..23: mol_type = other DNA
atttaggtgacactataagattt

SEQ ID NO: 65 - DNA, 26 bp, synthetic construct
  source 1..26: mol_type = other DNA
atttaggtgacactataagatttttt

SEQ ID NO: 66 - DNA, 32 bp, synthetic construct
  source 1..32: mol_type = other DNA
atttaggtgacactataagatttttttttttt

SEQ ID NO: 67 - DNA, 23 bp, synthetic construct
  source 1..23: mol_type = other DNA
atttaggtgacactataagaccc

SEQ ID NO: 68 - DNA, 26 bp, synthetic construct
  source 1..26: mol_type = other DNA
atttaggtgacactataagacccccc

SEQ ID NO: 69 - DNA, 32 bp, synthetic construct
  source 1..32: mol_type = other DNA
atttaggtgacactataagacccccccccccc

SEQ ID NO: 70 - DNA, 25 bp, synthetic construct
  source 1..25: mol_type = other DNA
gaatttaatacgactcactataggg

SEQ ID NO: 71 - DNA, 28 bp, synthetic construct
  source 1..28: mol_type = other DNA
gaatttaatacgactcactatagggaaa

SEQ ID NO: 72 - DNA, 31 bp, synthetic construct
  source 1..31: mol_type = other DNA
gaatttaatacgactcactatagggaaaaaa

SEQ ID NO: 73 - DNA, 34 bp, synthetic construct
  source 1..34: mol_type = other DNA
gaatttaatacgactcactatagggaaaaaaaaa

SEQ ID NO: 74 - DNA, 37 bp, synthetic construct
  source 1..37: mol_type = other DNA
gaatttaatacgactcactatagggaaaaaaaaaaaa

SEQ ID NO: 75 - DNA, 28 bp, synthetic construct
  source 1..28: mol_type = other DNA
gaatttaatacgactcactatagggttt

SEQ ID NO: 76 - DNA, 31 bp, synthetic construct
  source 1..31: mol_type = other DNA
gaatttaatacgactcactatagggtttttt

SEQ ID NO: 77 - DNA, 37 bp, synthetic construct
  source 1..37: mol_type = other DNA
gaatttaatacgactcactatagggtttttttttttt

SEQ ID NO: 78 - DNA, 28 bp, synthetic construct
  source 1..28: mol_type = other DNA
gaatttaatacgactcactatagggccc

SEQ ID NO: 79 - DNA, 31 bp, synthetic construct
  source 1..31: mol_type = other DNA
gaatttaatacgactcactatagggcccccc

SEQ ID NO: 80 - DNA, 37 bp, synthetic construct
  source 1..37: mol_type = other DNA
gaatttaatacgactcactatagggcccccccccccc

SEQ ID NO: 81 - DNA, 20 bp, synthetic construct
  source 1..20: mol_type = other DNA
aattaaccctcactaaaggg

SEQ ID NO: 82 - DNA, 23 bp, synthetic construct
  source 1..23: mol_type = other DNA
aattaaccctcactaaagggaaa

SEQ ID NO: 83 - DNA, 26 bp, synthetic construct
  source 1..26: mol_type = other DNA
aattaaccctcactaaagggaaaaaa

SEQ ID NO: 84 - DNA, 32 bp, synthetic construct
  source 1..32: mol_type = other DNA
aattaaccctcactaaagggaaaaaaaaaaaa

SEQ ID NO: 85 - DNA, 23 bp, synthetic construct
  source 1..23: mol_type = other DNA
aattaaccctcactaaagggttt

SEQ ID NO: 86 - DNA, 26 bp, synthetic construct
  source 1..26: mol_type = other DNA
aattaaccctcactaaagggtttttt

SEQ ID NO: 87 - DNA, 32 bp, synthetic construct
  source 1..32: mol_type = other DNA
aattaaccctcactaaagggtttttttttttt

SEQ ID NO: 88 - DNA, 23 bp, synthetic construct
  source 1..23: mol_type = other DNA
aattaaccctcactaaagggccc

SEQ ID NO: 89 - DNA, 26 bp, synthetic construct
  source 1..26: mol_type = other DNA
aattaaccctcactaaagggcccccc

SEQ ID NO: 90 - DNA, 32 bp, synthetic construct
  source 1..32: mol_type = other DNA
aattaaccctcactaaagggcccccccccccc

SEQ ID NO: 91 - DNA, 26 bp, synthetic construct
  source 1..26: mol_type = other DNA
  variation 26: note = H nucleotide can be repeated one or more times
gaatttaatacgactcactatarggh

SEQ ID NO: 92 - DNA, 24 bp, synthetic construct
  source 1..24: mol_type = other DNA
  variation 24: note = H nucleotide can be repeated one or more times
aattaaccctcactaaaaggrggh

SEQ ID NO: 93 - DNA, 21 bp, synthetic construct
  source 1..21: mol_type = other DNA
  variation 21: note = H nucleotide can be repeated one or more times
atttaggtgacactatargrh